(19) United States
(12) Patent Application Publication
(10) Pub. No.: US 2026/0237514 A1
(43) Pub. Date: Aug. 13, 2026

(54) MACHINE LEARNED ALGORITHMS FOR PATIENTS WITH SUSPECTED ACUTE CORONARY SYNDROME IN THE EMERGENCY DEPARTMENT

(71) Applicant: Universität Heidelberg, Heidelberg (DE)

(72) Inventors: Kiril Stoyanov, Heidelberg (DE); Evangelos Giannitsis, Heidelberg (DE); Christoph Reich, Heidelberg (DE); Norbert Frey, Kronshagen (DE); Hauke Hund, Heidelberg (DE); Hugo Katus, Heidelberg (DE); Andrea Horsch, Luzern Schweiz (CH); Gesa Albert, Rotkreuz (CH)

(21) Appl. No.: 19/156,093

(22) PCT Filed: Feb. 15, 2024
(86) PCT No.: PCT/EP2024/053900

§ 371 Date: Aug. 13, 2025

(30) Foreign Application Priority Data
Feb. 16, 2023 (EP) 23157003.7

(51) Int. Cl. G16H 50/30; G16H 10/60

(52) U.S. Cl. CPC G16H 50/30; G16H 10/60

(57) ABSTRACT

The present invention relates to methods for assessing subjects presenting with suspected acute coronary syndrome. Specifically, the present invention relates to a method for predicting of the risk of an adverse event in said subject. Further, the present invention relates to a method for predicting the need of myocardial revascularization of a patient presenting with suspected acute coronary syndrome. The methods of the present invention may be carried out as computer-implemented methods.

The present invention relates to methods for assessing subjects presenting with suspected acute coronary syndrome. Specifically, the present invention relates to a method for prediction of the risk of an adverse event in said subject. Further, the present invention relates to a method for predicting the need of myocardial revascularization of a patient presenting with suspected acute coronary syndrome. The methods of the present invention may be carried out as computer-implemented methods.

### BACKGROUND

In the United States, a total of 6.9 million patients visit an emergency department (ED) for chest pain (CP), and another 3.4 million for shortness of breath according to the National Hospital Ambulatory Medical Care Survey in 2014 (Stoyanov K M, Biener M, Hund H, Mueller-Hennessen M, Vafaie M, Katus H A, Giannitsis E. Effects of crowding in the emergency department on the diagnosis and management of suspected acute coronary syndrome using rapid algorithms: an observational study. BMJ Open. 2020 Oct. 8; 10(10):e041757. doi: 10.1136/bmjopen-2020-041757. PMID: 33033102; PMCID: PMC7545662). Only a fraction of these patients have a final diagnosis of ACS and require hospitalization and an invasive treatment strategy. The diagnostic workup requires admission to an ED, registration of a 12-lead electrocardiogram (ECG), a blood test to diagnose or to exclude myocardial injury, assessment of clinical symptoms and history, physical examination, and other diagnostic tests for diagnosis of ACS or differential diagnoses. Current 2020 European Society of Cardiology (ESC) guidelines (Collet J P, Thiele H, Barbato E, Barthélémy O, Bauersachs J, Bhatt D L, Dendale P, Dorobantu M, Edvardsen T, Folliguet T, Gale C P, Gilard M, Jobs A, Jüni P, Lambrinou E, Lewis B S, Mehilli J, Meliga E, Merkely B, Mueller C, Roffi M, Rutten F H, Sibbing D, Siontis G C M; ESC Scientific Document Group. 2020 ESC Guidelines for the management of acute coronary syndromes in patients presenting without persistent ST-segment elevation. Eur Heart J. 2020 Aug. 29:ehaa575. doi: 10.1093/eurheartj/ehaa575. Epub ahead of print. PMID: 32860058) recommend monitoring of patients for ECG and vital signs, unless a myocardial injury has been ruled out.

At present, the numbers of ED (emergency department) visits for unspecific chest pain are increasing globally causing overcrowding in busy EDs. ED physicians have to apply strict admission criteria in order to cope with a relative shortage of hospital beds. Therefore, accurate diagnosis followed by risk stratification are mandatory to guide the decision for hospitalization or discharge, as well as to select the appropriate selective-invasive strategy, i.e. the identification of patients who should undergo an invasive strategy rather than anatomic imaging or functional testing for myocardial ischemia. While the decision to admit and to allocate to an invasive strategy is unequivocal in patients classified as rule-in, sparse information exist for patients classified as rule-out and for most patients in the observe zone. This situation has even been aggravated since the 2020 ESC Guidelines shifted patients previously assigned to an intermediate risk with planned invasive strategy within 72 hours to the low risk category.

In the studies underlying the present invention, different methods were developed which improve the assessment of patients presenting suspected ACS (see also Examples section). The methods are herein referred to as “prognostic method” and “predictive method”. The developed methods allow for a) predicting the risk of an adverse event and b) for predicting the need of myocardial revascularization of a patient presenting with suspected acute coronary syndrome.

As described in the Examples section, the developed methods are comprehensive providing valuable aid for two major challenges. Specifically, they enable a more accurate estimation of individual risk for death at 365 days and at 180 days than the GRACE score which is the preferred clinical risk stratification tool (Level of evidence IIA) promoted by the 2020 ESC guidelines. Accordingly, the ED physician can use a better risk prediction tool to justify his/her decision for discharge with or without the recommendation for further outpatient diagnostic workup. Further, even patients with unstable angina who are classified as ruleout and are regarded as being at low risk for major coronary events may have an underlying obstructive coronary artery disease that necessities coronary intervention. At present, tools that provide information on the likelihood of CAD (Coronary artery disease) are sparse. The ESC Guidelines on Chronic Coronary Syndromes recommend the use of the ESC Consortium algorithm, a modified Diamond-Forrester algorithm which has a moderate ability to identify patients at risk for a significant coronary artery stenosis (Genders T S, et al. BMJ: British Medical Journal 2012 June 12, 344: e3485). As such, the developed method provides better estimation on the pretest probability for CAD requiring revascularization that may facilitate the decision for invasive strategy as compared to coronary CT or functional stress testing.

### Prognostic Method

The study cohort consists of all consecutive patients who presented with suspected ACS at the Chest Pain Unit of the University Hospital of Heidelberg between 30 Jun. 2016 until 1 Jul. 2018, and had been triaged into “rule-out” or “observe zone” following retrospective adjudication by three cardiologists that were not involved in the management of the patient. Patients triaged as “rule-in” and those with STEMI were excluded. 3,928 patients were considered eligible. The outcome variable included only all-cause death occurring within 180 days or 365 days in order to allow a larger number of outcome events in a cohort of patients with low all-cause mortality. The cohort was stratified by the outcome of interest and then randomly split into a training set comprising 75% (n=2,946) of the entire study population and a 25% test set (n=982; stratified random sampling). We used logistic regression with elastic net regularization to fit a model to the training data. all estimated parameters were collected strictly on the training data set only. Since we used mean imputation for numeric variables and mode imputation for nominal variables (only 0.37% of data entries were missing in total, n=343 data points), mean and mode imputation values were trained on the training data set only. We did not perform imputations on the outcome variable. Skewed variables were log transformed and all numeric variables were centered and scaled based on the distribution of the training data. Further, we considered the interaction term between patient's baseline Troponin and patient's age as relevant and added it to the model. Using 5-fold cross validation we estimated the hyperparameters penalty (represents the amount of penalty applied to logistic regression model) and mixture (represents the relative amount of penalties L1 and L2) needed for elastic net regularization. The hyperparameter grid consisted of 1,000 parameter combinations each of which was fitted in the model selection process during the 5-fold cross validation. The best fitted hyperparameters were evaluated based on performance of ROC-AUC on the 5-fold cross validation. After finding the best hyperparameters we finally fit the model on the whole training data set to obtain estimates of the covariates. The regularization technique may result in some estimates being 0 and therefore no longer relevant for the calculation of the outcome probability. Due to the unbalanced outcome occurring in our dataset we chose ROC-AUC to be our primary performance measure. We then calculated the Youden-Index on the ROC curve to obtain a threshold for reporting sensitivity and specificity of our models.

The minimal model consisted of five variables: initial Troponin, delta Troponin, age, gender, creatinine. The full model consisted of ten variables: initial Troponin (numeric), delta Troponin (numeric), age (numeric), gender (binary), creatinine (numeric), abnormal ECG (binary), CRP value (numeric), Sodium (numeric). Hemoglobine (numeric) and thrombocytes value (numeric). Besides the minimal and the full model, another 31 models with six, seven, eight, nine or ten parameters were constructed adding parameters to the minimal model such as six, seven, eight, nine or ten parameters, or by exchange of related variables with a similar prognostic information such as but not limited to urea or estimated glomerular filtration rate instead of creatinine as indicators of renal function. All models including the minimal and the full model contain the minimal set of aforementioned variables. In total 33 models were constructed and evaluated on a blinded test set that comprised 25% of the entire population.

There are methods to predict short- and intermediate term outcomes such as death in patients with suspected ACS. There is need to improve risk stratification because the overall performance of established clinical scores is suboptimal. The use of clinical scores was found particularly less helpful when hs-cTn assays were used in combination with accelerated protocols. When the ESC 0/1 hour protocol or the High-STEACS pathway is being used, several clinical scores did not improve accuracy or safety of the fast protocol but decreased the numbers of eligible patients considerably. Given that commonly patients who are triaged as “rule-in” are viewed as high-risk patients with a high pre-test probability for acute myocardial injury or acute myocardial infarction only patients categorized as “rule-out” or into the “observe zone” were targeted for the prediction tool. In the studies underlying the present invention, a method for estimation of risk for all-cause death at 365 days (and 180 days) was established, since sufficient numbers of events occurred within 365 days (n=100) and within 180 days (n=65). The method is based on imputation of paired high-sensitivity cardiac troponin concentrations and concentration change of cardiac troponin T in the second blood draw. Other variables include, but are not limited to age, sex, past medical history, present symptoms, vital signs, ECG parameters, and other laboratory values.

Accordingly, the present invention relates to a computer-implemented method for predicting the risk of an adverse event of a patient presenting with suspected acute coronary syndrome, comprising the steps of
a) receiving data for a set of parameters obtained from the patient at a processing unit, wherein said set of parameters comprises at least five, such as five, six, seven, eight, nine or ten of the following parameters
   i. the amount of a cardiac Troponin in a first sample obtained from said patient at presentation,
   ii. the amount of said cardiac Troponin in a second sample obtained from said patient within 30 minutes to 6 hours after the first sample, such as within 1 hour to 3 hours after the first sample, such as within 1 hour to 2 hours after the first sample,
   iii. the amount of CRP (C-reactive protein) in a sample from the patient,
   iv. at least one parameter for the patient's renal function selected from the group consisting of the amount of urea in a sample from the patient, the patient's GFR and the patient's serum creatinine amount, in particular the patient's serum creatinine amount.
   v. the amount of sodium in a sample from the patient,
   vi. the amount of hemoglobin in a sample from the patient,
   vii. the patient's thrombocyte level,
   viii. the patient's age,
   ix. the patient's gender, and
   x. the presence or absence of a normal ECG in said patient,
b) carrying out at the processing unit an analysis of the set of parameters, wherein said analysis comprises calculating a score for predicting the risk of an adverse event of said patient based on the set of parameters received in step a), and
c) providing information on the score calculated in step b), thereby predicting the risk of an adverse event.

In one embodiment, data on at least five, such as five, six, seven, eight, nine or ten of the following parameters are received: the amount of a cardiac Troponin in a first sample obtained from said patient at presentation; the amount of said cardiac Troponin in a second sample (as set forth above); the amount of CRP (C-reactive protein) in a sample from the patient, the patient's serum creatinine amount, the amount of sodium in a sample from the patient, the amount of hemoglobin in a sample from the patient, the patient's thrombocyte level, information on the patient's age, information on the patient's gender, and information on the presence or absence of a normal ECG in said patient (i.e. information on whether the patient has a normal or abnormal ECG).

In one embodiment, the parameters are the parameters from any one of models 1 to 33 shown in Table 3 or 4 in the Examples section.

In a preferred embodiment, data on at least the five following parameters are obtained: the amount of a cardiac Troponin in a first sample obtained from said patient at presentation; the amount of said cardiac Troponin in a second sample (as set forth above), information on the patient's age, information on the patient's gender and the patient's serum creatinine amount.

Alternatively, the present invention relates to a method of predicting the risk of an adverse event of a patient presenting with suspected acute coronary syndrome, comprising the steps of
a) carrying out at least five, such as five, six, seven, eight, nine or ten of the following steps a1) to a10):
   a1) determining the amount of a cardiac Troponin in a first sample obtained from said patient at presentation,
   a2) determining the amount of said cardiac Troponin in a second sample obtained from said patient within 30 minutes to six hours after the first sample, such as within 1 hour to 3 hours after the first sample, such as within 1 hour to 2 hours after the first sample,
   a3) determining the amount of CRP (C-reactive protein) in a sample from the patient,
   a4) determining at least one parameter for the patient's renal function selected from the group consisting of the amount of urea nitrogen in a sample from the patient, the patient's GFR and the patient's serum creatinine amount, preferably the patient's serum creatinine amount,
   a5) determining the amount of sodium in a sample from the patient,
   a6) determining the amount of hemoglobin in a sample from the patient,
   a7) determining the patient's thrombocyte level in a sample from the patient
   a8) providing information on the patient's age,
   a9) providing information on the patient's gender, and
   a10) providing information on the presence or absence of a normal ECG in said patient,
b) calculating a score for predicting the risk of an adverse event of said patient based on the information obtained in step a), and
c) predicting the risk of the patient of an adverse event based on the score calculated in step b).

Preferred combinations for the steps under a) are described above in connection with the computer-implemented method.

In a preferred embodiment of the aforementioned methods, the sample is a blood, serum or plasma sample.

In a preferred embodiment of the aforementioned methods, the adverse event is death, such as all cause death.

In a preferred embodiment of the aforementioned methods, the risk of an adverse event within about 180 to about 365 days is predicted. For example, the risk of death within about 180 days is predicted. Alternatively, the risk of death within about 180 days is predicted.

### Predictive Method

The study cohort consists of all consecutive patients who presented with suspected ACS at the Chest Pain Unit of the University Hospital of Heidelberg between 30 Jun. 2016 until 1 Jul. 2018, and received coronary angiography with or without myocardial revascularization within 30 days after index presentation. Patients with STEMI were excluded. 1,344 patients were considered eligible. The outcome variable included patients with a significant coronary stenosis of 50% luminal obstruction or more that were allocated to percutaneous coronary intervention (PCI), coronary bypass surgery (CABG), or who were treated conservatively because of attempted but failed or unsuccessful PCI or a complex coronary anatomy that was deemed unsuitable for myocardial revascularization. Machine-learning enabled models to predict the presence of significant coronary artery disease requiring revascularization were trained using logistic regression with elastic net regularization.

The cohort was stratified by the outcome of interest and then randomly split into a training set comprising 75% (n=1,007) of the entire study population and a 25% test set (n=337; stratified random sampling). All estimated parameters were collected strictly on the training data set only. Since we used mean imputation for numeric variables and mode imputation for nominal variables (only 2.62% of data entries were missing in total, n=670 data points), mean and mode imputation values were trained on the training data set only. The outcome variable “Obstructive CAD requiring revascularization” was present in n=889 and was not present in n=444 patients. There were no missing values for the outcome variable. Skewed variables were log transformed and all numeric variables were centered and scaled based on the distribution of the training data. Further, we considered the interaction term between patients' baseline Troponin and patients' age as relevant and added it to the model. Using 5-fold cross validation we estimated the hyperparameters penalty (represents the amount of penalty applied to logistic regression model) and mixture (represents the relative amount of penalties L1 and L2) needed for elastic net regularization. The hyperparameter grid consisted of 1,000 parameter combinations each of which was fitted in the model selection process during the 5-fold cross validation. The best fitted hyperparameters were evaluated based on performance of ROC-AUC on the 5-fold cross validation. After finding the best hyperparameters we finally fit the model on the whole training data set to obtain estimates of the covariates. We chose ROC-AUC to be our primary performance measure. We then calculated the Youden-Index on the ROC curve to obtain a threshold for reporting sensitivity and specificity of our models. The minimal model consisted of eight variables: initial Troponin, delta Troponin, age, gender, creatinine, smoking status, history of revascularization and experienced chest pain. The full model included the following variables:
initial Troponin (numeric),
delta Troponin (numeric),
age (numeric), gender (binary),
creatinine (numeric), alternatively or additionally CKD-EPI (numeric) and/or urea (numeric),
abnormal ECG (binary),
cardiac risk factors (diabetes mellitus (binary),
smoking status (binary),)
history of coronary artery disease (binary), and
leading symptom dyspnea (binary).
   Background for the Need of ML Models to Predict Obstructive CAD Requiring Revascularization within 30 Days

A challenge for physicians is the decision to perform a coronary angiography. The risk of unnecessary coronary angiography that is associated with excessive risk of procedure and non-procedure related major bleedings, radiation exposure and potential kidney injury has to be balanced against the risk to miss a severe coronary artery disease that would benefit from a reperfusion therapy. For this purpose, ESC guidelines² recommend a selectively invasive strategy for low risk patients who remain free of recurrent symptoms. Moreover, the recommendation to perform stress testing preferably using imaging stress tests to decide whether a low risk patient should undergo a routine coronary angiography (selective-invasive strategy) is practically not feasible, given the high numbers of patients that would require specialized imaging stress testing.

Currently, there is no established predictor or model that allows a reliable prediction of an obstructive coronary artery disease requiring revascularization. As a consequence, there is potential overuse of coronary angiography in low risk patients and potential underuse in patients with equivocal risk, e.g. patients with unstable angina where the decision for selective invasive strategy is based on the re-occurrence of symptoms despite optimal medical therapy, or on pathological stress test, preferably stress imaging. Other scenarios include patients with equivocal symptoms and comorbidities including heart failure, obstructive airways disease, and arterial hypertension.

In the studies underlying the present invention, a method for estimation of the probability of having an obstructive coronary artery disease requiring reperfusion therapies within 30 days after index admission was established. The method helps to predict the likelihood of an obstructive coronary artery disease requiring revascularization. This method is based on characteristics of the patient including, but not limited to, age, sex, past medical history, present symptoms, vital signs, ECG parameters, hs-cTnT, hs-cTnT kinetics and/or other laboratory values. Read-out is the percent probability for the predicted event.

Accordingly, the present invention relates to a computer-implemented method for predicting the need of myocardial revascularization of a patient presenting with suspected acute coronary syndrome, comprising the steps of
a) receiving data for a set of parameters obtained from the patient at a processing unit, wherein said set of parameters comprises at least eight, such as eight, nine, ten, eleven, twelve, thirteen, fourteen or fifteen of the following parameters:
   i. the amount of a cardiac Troponin in a first sample obtained from said patient at presentation,
   ii. the amount of said cardiac Troponin in a second sample obtained from said patient within 30 minutes to 6 hours after the first sample, such as within 1 hour to 3 hours after the first sample, such as within 1 hour to 2 hours after the first sample,
   iii. the patient's age,
   iv. the patient's gender,
   v. a parameter for the patient's renal function selected from the group consisting of the patient's GFR (such as the GFR according to the Chronic Kidney Disease Epidemiology Collaboration (CKD-EPI) formula), the patient's serum creatinine amount, and the patient's blood urea nitrogen (BUN) amount, in particular the patient's serum creatinine amount,
   vi. the presence or absence of chest symptoms, such as chest pain, in said patient,
   vii. the presence or absence of dyspnea in said patient (preferably as leading symptom),
   viii. the presence or absence of a normal ECG in said patient, and
   ix. information on the patient's past medical history, comprising at least one, preferably all, of the following: information on the patient's history of diabetes, patient's history of nicotine smoking, information on the patient's history of coronary artery disease,
b) carrying out at the processing unit an analysis of the set of parameters, wherein said analysis comprises calculating a score for predicting the need of myocardial revascularization of said patient based on the set of parameters received in step a), and
c) providing information on the score calculated in step b), thereby predicting the need of myocardial revascularization of said patient.

In one embodiment, data on at least five, such as five, six, seven, eight, nine or ten of the following parameters are received: the amount of a cardiac Troponin in a first sample obtained from said patient at presentation; the amount of said cardiac Troponin in a second sample (as set forth above); the patient's serum creatinine amount, the presence or absence of chest symptoms in said patient, the presence or absence of dyspnea in said patient, information on the patient's sex (gender), information on the patient's age, information on the patient's past medical history, comprising at least one, preferably all, of the following: information on the patient's history of diabetes, information on the patient's history of nicotine smoking, information on the patient's history of coronary artery disease, and the presence or absence of a normal ECG in said patient (i.e. information on whether the patient has a normal or abnormal ECG).

In one embodiment, the parameters are the parameters from any one of models 1 to 33 in Table 9 in the Examples section.

In a preferred embodiment, data on at least the eight following parameters are obtained: the amount of a cardiac Troponin in a first sample obtained from said patient at presentation; the amount of said cardiac Troponin in a second sample (as set forth above); information on the patient's sex (gender), information on the patient's age, information on the patient's history of coronary artery disease, information on the patient's history of nicotine smoking, and information on the presence or absence of chest symptoms in said patient (such as chest pain).

Further, the present invention relates to a method of predicting the need of myocardial revascularization of a patient presenting with suspected acute coronary syndrome, comprising
a) carrying out at least eight, such as eight, nine, ten, eleven, twelve, thirteen, fourteen or fifteen of the following steps a1) to a9):
   a1) determining the amount of a cardiac Troponin in a first sample obtained from said patient at presentation,
   a2) determining the amount of said cardiac Troponin in a second sample obtained from said patient within 30 minutes to six hours after the first sample, such as within 1 hour to 3 hours after the first sample, such as within 1 hour to 2 hours after the first sample,
   a3) providing information on the patient's age,
   a4) providing information on the patient's gender,
   a5) providing information on a parameter for the patient's renal function selected from the group consisting of the patient's GFR, the patient's serum creatinine amount, and the patient's blood urea nitrogen (BUN) amount, in particular the patient's serum creatinine amount
   a6) providing information on the presence or absence of chest symptoms in said patient,
   a7) providing information on the presence or absence of dyspnea in said patient,
   a8) providing information on the presence or absence of a normal ECG in said patient,
   a9) providing information on the patient's past medical history, comprising at least one, preferably all, of the following: information on the patient's history of diabetes, information on the patient's history of nicotine smoking, and information on the patient's history of coronary artery disease,
b) calculating a score for predicting the need of myocardial revascularization of said patient based on the information obtained in step a) and
c) predicting the need of myocardial revascularization of the patient event based on the score calculated in step b).

Preferred combinations for the steps under a) are described above in connection with the computer-implemented method.

In a preferred embodiment of the aforementioned methods, the sample is a blood, serum or plasma sample.

In a preferred embodiment of the aforementioned methods, the myocardial revascularization is due to obstructive coronary artery disease.

In a preferred embodiment of the aforementioned methods, the need of myocardial revascularization within about 30 days is predicted.

In a preferred embodiment of the aforementioned methods, the score is indicative for the likelihood of the patient to require myocardial revascularization.

In a preferred embodiment of the aforementioned methods, the calculated score is shown on a display.

In a preferred embodiment, the above methods comprise recommending or subjecting the patient to myocardial revascularization.

### Definitions

As the forth above, the present invention encompasses two methods, a prognostic method, and a predictive method. The definitions and explanations provided herein above shall apply to all methods, except if specified otherwise.

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person under-stands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, and even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

It will be understood that the methods according to the present invention are, preferably, ex-vivo methods, i.e. they do not require to be practiced on the human or animal body. Rather, the methods are based on existing patient data previously gathered. For example, it is envisaged that the methods are in vitro methods. Moreover, they may comprise steps in addition to those explicitly mentioned above. For example, further steps may relate to sample pre-treatments or evaluation of the results obtained by the method. The method may be carried out manually or assisted by automation.

In some embodiments, the methods of the present invention are computer-implemented methods. In computer-implemented methods, typically, all steps of the computer-implemented method of the present invention are performed by one or more processing units of a computer or a computer network.

The phrase “predicting the risk of an adverse event” as used means that the subject to be analyzed by the method of the present invention is allocated either into the group of subjects being at risk of suffering from an adverse event or into the group of subjects not being at risk of suffering from said adverse event. Thus, it is predicted whether the subject is at risk or not at risk of an adverse event. As used herein “a patient who is at risk of an adverse event”, preferably has an elevated risk of suffering from said adverse event, preferably, within the predictive window. Preferably, said risk is elevated as compared to the average risk in a cohort of subjects. As used herein, “a subject who is not at risk of an adverse event”, preferably, has a reduced risk for developing said adverse event, preferably, within the predictive window. Preferably, said risk is reduced as compared to the average risk in a cohort of subjects. Preferably, an elevated risk or a reduced risk as referred to herein is a statistically significant elevated or reduced risk. More preferably, the predictive window in accordance with the present invention for which the risk of an adverse event is predicted is within about 180 to about 365 days, such as within about 180 days, or within about 365 days, i.e. within one year The predictive window is, typically, calculated starting from the day on which the parameters have been obtained from the patient.

The term “adverse event” as used herein refers to any worsening which occurs in the patient within the predictive window and which severely and negatively affects one or more physiological functions within said patient. More specifically, a physiological function of the cardiovascular system shall become affected. More preferably, said adverse event shall be death of any cause. Thus, in this embodiment, the adverse event is death. The term “death” as used herein, preferably, relates to death from any cause.

The phrase “predicting the need of myocardial revascularization” as used means that the subject to be analyzed by the method of the present invention is allocated either into the group of subjects being in need of myocardial revascularization or into the group of subjects not being in need of myocardial revascularization. Thus, it is predicted whether the subject is in need, or not, of myocardial revascularization. As used herein “a patient who is in need of myocardial revascularization”, preferably has an elevated likelihood of needing myocardial revascularization, preferably, within the predictive window. Preferably, said likelihood is elevated as compared to the average likelihood for need of myocardial revascularization in a cohort of subjects. As used herein, “a subject who is not in need of myocardial revascularization”, preferably, has a reduced likelihood for needing myocardial revascularization, preferably, within the predictive window. Preferably, said likelihood is reduced as compared to the average likelihood for need of myocardial revascularization in a cohort of subjects. Preferably, an elevated or a reduced likelihood as referred to herein is a statistically significant elevated or reduced likelihood. More preferably, the predictive window in accordance with the present invention for which need of myocardial revascularization is predicted is within about 30 days. The predictive window is, typically, calculated starting from the day on which the parameters have been obtained from the patient. Alternatively, obstructive coronary artery disease may be diagnosed by the method of the invention.

The term “myocardial revascularization” as used herein refers to any therapeutic measure which allows for revascularization of tissue affected by obstructive vessel events such as those caused by cardiovascular diseases or disorders and, preferably, by obstructive coronary artery disease. Coronary artery disease as referred to herein is, preferably, defined as any luminal obstruction of a major epicardial coronary artery of 50% or more. Preferably, therapeutic measures which allow for myocardial revascularization in accordance with the present invention are either percutaneous coronary intervention with or without stenting or coronary arterial bypass grafting (CABG).

Furthermore, the myocardial revascularization is one of the following:
a. invasive coronary angiography with percutaneous coronary intervention with or without stenting,
b. invasive coronary angiography with lesion(s) suitable for revascularization and attempted percutaneous coronary intervention with or without stenting,
c. invasive coronary angiography with lesion(s) suitable for revascularization and attempted but failed or unsuccessful percutaneous coronary intervention,
d. invasive coronary angiography with relevant lesion(s) unsuitable for revascularization and subsequently conservative medical treatment without percutaneous coronary intervention,
e. invasive coronary angiography with lesion(s) suitable for revascularization and immediate transfer for coronary bypass surgery
f. invasive coronary angiography with lesion(s) suitable for revascularization and recommendation for coronary bypass surgery, or
g. invasive coronary angiography with lesion(s) planned for revascularization, such as percutaneous coronary intervention or coronary bypass surgery.

In a preferred embodiment, the myocardial revascularization is invasive coronary angiography with percutaneous coronary intervention with or without stenting.

In another preferred embodiment, the myocardial revascularization is or coronary arterial bypass grafting (CABG).

In another preferred embodiment, the treatment is conservative pharmacological treatment, such as administration of an effective amount of acetylsalicylic acid, at least one beta blocker, at least one angiotensin II receptor blocker (ARBs), and/or at least one statin, in case of failed or unsuccessful percutaneous coronary intervention, or if lesion(s) are relevant but unsuitable for percutaneous coronary intervention or coronary bypass surgery.

As will be understood by those skilled in the art, the aforementioned assessments made by the methods of the present invention, i.e. the prediction or prognosis, are usually not intended to be correct for 100% of the investigated individuals. The term typically requires that the assessment is correct for a statistically significant portion of the individuals (e.g., a cohort in a cohort study). Whether a value indicating a difference in risk or likelihood, a portion of a cohort or any other difference in values is statistically significant can be determined without further ado by the person skilled in the art using various well-known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001.

The term “sample” refers to a sample of a body fluid, to a sample of separated cells or to a sample from a tissue or an organ which is known or suspected to comprise an analyte which needs to be determined as a parameter. It will be understood that the sample may depend on the analyte to be determined. For example, if a cardiac Troponin shall be determined in a first and/or second sample as referred to herein, said sample may be typically a sample containing or suspected to contain said cardiac Troponin. Typical samples may be whole blood samples or derivatives thereof such as plasma or serum samples. For other analytes, the sample may be urine samples as well or other body fluids or cell or tissue samples. The skilled artisan is well aware which samples can be used for a given analyte in order to determine the parameter referred to in accordance with the present invention. Moreover, the skilled person is also well aware of how such samples can be taken from the patient, e.g., by conventional blood taking equipment such as lancets, biopsies or the like.

In a preferred embodiment, the sample is blood, serum or plasma sample.

In another preferred embodiment, the sample is interstitial fluid.

The term “cardiac Troponin” typically refers to human cardiac Troponin T or cardiac Troponin I. The term, however, also compasses variants of the aforementioned specific Troponins, i.e., preferably, of cardiac Troponin I, and more preferably, of cardiac Troponin T. Such variants have at least the same essential biological and immunological properties as the specific cardiac Troponins. In particular, they share the same essential biological and immunological properties if they are detectable by the same specific assays referred to in this specification, e.g., by ELISA Assays using polyclonal or monoclonal antibodies specifically recognizing the said cardiac Troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least about 50%, at least about 60%, at least about 70%, at least about 80%, at least about 85%, at least about 90%, at least about 92%, at least about 95%, at least about 97%, at 10 least about 98%, or at least about 99% identical with the amino sequence of the specific Troponin. Variants may be allelic variants or any other species specific homologs, paralogs, or orthologs. Moreover, the variants referred to herein include fragments of the specific cardiac Troponins or the aforementioned types of variants as long as these fragments have the essential immunological and biological properties as referred to above. Preferably, the cardiac troponin variants have immunological properties (i.e. epitope composition) comparable to those of human troponin T or troponin I. Thus, the variants shall be recognizable by the aforementioned means or ligands used for determination of the concentration of the cardiac troponins. Thus, the variants shall be recognizable by the aforementioned means or ligands used for determination of the concentration of the cardiac troponins. Such fragments may be, e.g., degradation products of the Troponins. Further included are variants which differ due to posttranslational modifications such as phosphorylation or myristylation. Preferably the biological property of troponin I and its variant is the ability to inhibit actomyosin ATPase or to inhibit angiogenesis in vivo and in vitro, which may e.g. be detected based on the assay described by Moses et al. 1999 PNAS USA 96 (6): 2645-2650). Preferably the biological property of troponin T and its variant is the ability to form a complex with troponin C and I, to bind calcium ions or to bind to tropomyosin, preferably if present as a complex of troponin C, I and T or a complex formed by troponin C, troponin I and a variant of troponin T. Troponin T or Troponin I can be determined by immunoassays, e.g., ELISAs, that are well known in the art and commercially available. Particular preferred in accordance with the present invention is the determination of Troponin T with high sensitivity using, e.g. a commercially available hs-cTn assay.

CRP (C-reactive protein) is an acute phase protein that was discovered more than 75 years ago to be a blood protein that binds to the C-polysaccharide of pneumococci. CRP is known as a reactive inflammatory marker and is produced by a distal organ (i.e. the liver) in response or reaction to chemokines or interleukins originating from the primary lesion site. CRP is known to consist of five single subunits, which are non-covalently linked and assem-30 bled as a cyclic pentamer with a molecular weight of approximately 110-140 kDa. Preferably, CRP as used herein relates to human CRP. The sequence of human CRP is well known and disclosed, e.g., by Woo et al. (J. Biol. Chem. 1985. 260 (24), 13384-13388). The level of CRP is usually low in normal individuals but can rise 100- to 200-fold or higher due to inflammation, infection or injury (Yeh (2004) Circulation. 2004; 109:11-11-11-14). It is known that CRP is an independent factor for the prediction of a cardiovascular risk. CRP can be determined by immunoassays, e.g., ELISAs, that are well known in the art and are commercially available. Typically, CRP is hsCRP (high sensitive CRP).

Urea is the major end product of protein nitrogen metabolism. It has the chemical formula CO(NH₂)₂ and is synthesized by the urea cycle in the liver from ammonia which is produced by amino acid deamination. Urea is excreted mostly by the kidneys but minimal amounts are also excreted in sweat and degraded in the intestines by bacterial action. Determination of blood urea nitrogen is the most widely used screening test for renal function. Urea can be measured by an in vitro test for the quantitative determination of urea/urea nitrogen in human serum, plasma and urine on Roche/Hitachi cobas c systems. The test can be carried out automatically using different analysers including cobas c 311 and cobas c 501/502. The assay is a kinetic assay with urease and glutamate dehydrogenase. Urea is hydrolyzed by urease to form ammonium and carbonate. In the second reaction 2-oxoglutarate reacts with ammonium in the presence of glutamate dehydrogenase (GLDH) and the coenzyme NADH to produce L-glutamate. In this reaction 2 moles of NADH are oxidized to NAD+ for each mole of urea hydrolyzed. The rate of decrease in the NADH concentration is directly proportional to the urea concentration in the specimen and is measured photometrically.

In some embodiments, the amount of urea can be determined. Alternatively, the amount of blood urea nitrogen (abbreviated BUN) can be determined.

The marker “creatinine” is well known in the art. In muscle metabolism, creatinine is synthesized endogeneously from creatine and creatine phosphate. Under conditions of normal renal function, creatinine is excreted by glomerular filtration. Creatinine determinations are performed for the diagnosis and monitoring of acute and chronic renal disease as well as for the monitoring of renal dialysis. Creatinine concentrations in urine can be used as reference values for the excretion of certain analytes (albumin, α-amylase). Creatinine can be determined as described by Popper et al., (Popper H et al. Biochem Z 1937; 291:354), Seelig and Wüst (Seelig H P, Wüst H. Ärztl Labor 1969; 15:34) or Bartels (Bartels H et al. Clin Chim Acta 1972; 37:193). Preferably, the amount of creatinine is determined in a serum sample. Thus, the patient's serum creatinine amount is determined.

The term “hemoglobin” as used herein, preferably, refers to total hemoglobin. The level of Hemoglobin can be measured by well-known methods, e.g. by oxidation of hemoglobin to methemoglobin by potassium hexacyanoferrate. The hemoglobin level is proportional to the color intensity and, e.g., can be measured at a wavelength of 567 nm and 37° C. The level of hemoglobin can be also measured by contacting the sample with an antibody which specifically binds to hemoglobin.

The parameter “GFR (glomerular filtration rate)” is a well-known parameter which can be determined by clinical chemistry assays and detection methods well known in the art. GFR may be accurately calculated by comparative measurements of substances in the blood and urine, or estimated by formulas using just a blood test result (eGFR). Usually these estimates are used in clinical practice in particular in elderly and sick patients where reliable urine collections are difficult. eGFR is associated with GFR For clinical assessment scales of eGFR and GFR can be used interchangeably. In the studies underlying the present invention, the eGFR was determined. In an embodiment, the GFR is the GFR according to the Chronic Kidney Disease Epidemiology Collaboration (CKD-EPI) formula.

The same applies for the amount of “sodium”. The amount of sodium can also be determined without further ado by using routine clinical chemistry and well known detection techniques.

Further, the thrombocyte level can be determined by well-established clinical laboratory analyses. For example, the respective cells may be counted manually in a counting chamber. Alternatively, automation equipment including FACS analyzers may be used.

The term “amount” as used herein refers to the absolute amount of a compound referred to herein, the relative amount or concentration of the said compound as well as any value or parameter which correlates thereto or can be derived therefrom. Such values or parameters comprise intensity signal values from all specific physical or chemical properties obtained from the said compounds by direct measurements, e.g., intensity values in mass spectra or NMR spectra. Moreover, encompassed are all values or parameters which are obtained by indirect measurements specified elsewhere in this description, e.g., response levels determined from biological read out systems in response to the compounds or intensity signals obtained from specifically bound ligands. It is to be understood that values correlating to the aforementioned amounts or parameters can also be obtained by all standard mathematical operations.

The terms “determining” or “measuring” the level of a marker as referred to herein refers to the quantification of the biomarker, e.g. to determining the level of the biomarker in the sample, employing appropriate methods of detection described elsewhere herein. In an embodiment, the level of the at least one biomarker is measured by contacting the sample with a detection agent that specifically binds to the respective marker, thereby forming a complex between the agent and said marker, detecting the level of complex formed, and thereby measuring the level of said marker.

Electrocardiography (abbreviated ECG) is the process of recording the electrical activity of the heart by suitable ECG. An ECG device records the electrical signals produced by the heart which spread throughout the body to the skin. The recording is of the electrical signal is achieved by contacting the skin of the test subject with electrodes comprised by the ECG device. The process of obtaining the recording is non-invasive and risk-free. In some embodiments, of the method of the present invention it is assessed whether, the patient has a normal ECG, or not. Thus, the presence or absence of a normal ECG in the patient is assessed.

An ECG is considered as normal, if all of the following criteria are met:
1. Sinus rhythm without second or third degree AV-Block AND
2. No bundle branch block (LBBB, RBBB) or unspecific block (QRS>=120 ms) AND
3. No ventricular pacing (no single RV/LV paced QRS in obtained EKG) AND
4. No ST-depression >1 mV (in >=2 contiguous leads) AND
5. No T-wave inversions (in >=2 contiguous leads)

If at least one of the above criteria is not met, the ECG of the patient is not normal.

In some embodiments, information on the presence or absence of chest symptoms in said patient is taken into account for the score. Thus, it is assessed whether the subject shows chest symptoms, or not. In particular, it is assessed whether the subject suffers from chest pain, or not.

In some embodiments, information on the presence or absence of dyspnea (“Shortness of breath”) in the patient is taken into account for the score. The term “dyspnea” refers to an impaired respiration which results in an increased respiratory frequency and/or an increased respiratory volume. Thus, shortness of breath may result, preferably, in hyperventilation. Shortness of breath occurs, usually, at an oxygen saturation level below the normal oxygen saturation level of at least 95%. As used herein, the term “dyspnea” refers to acute shortness of breath, i.e. a non-permanently occurring shortness of breath.

In some embodiments, the method of the present invention encompasses obtaining information on the patient's past medical history. Said information may be e.g. obtained from the patient's medical records. In some embodiments, the information comprises information on the patient's history of diabetes. Thus, it is assessed whether the patient is suffering or has suffered from diabetes. The term “diabetes” as used herein refers, preferably, to diabetes mellitus type I or diabetes mellitus type II. The symptoms and clinical parameters associated with diabetes mellitus type I and II are well known in the art. In a preferred embodiment, it is assessed whether the patient is suffering or has suffered from diabetes mellitus type II.

In some embodiments, the method of the present invention encompasses obtaining information comprises information on the patient's history of nicotine smoking. The term “smoking” as used herein refers, preferably to previous or current smoking. In a preferred embodiment, it is assessed whether the patient is actively smoking or smoked nicotine in the past.

In some embodiments, the information comprises information on the patient's history of coronary artery disease, for example CAD with or without history of myocardial revascularization. Typically, it is assessed whether the patient suffers from coronary artery disease (CAD), or not. A patient who has a history of coronary artery disease preferably fulfills at least one of the following criteria: previous myocardial infarction, known CAD, previous percutaneous coronary intervention (PCI) and/or previous coronary bypass surgery (CABG).

In some embodiments, the information on coronary artery disease comprises information on the patient's history of myocardial revascularization. Thus, it is assessed whether the patient underwent a myocardial revascularization in the past, such as PCI and or CABG. The term “myocardial revascularization” is defined elsewhere herein.

In some embodiments, the information comprises information on the patient's history of myocardial infarction. Thus, it is assessed whether the patient has suffered from a myocardial infarction in the past. The term “myocardial infarction” is defined elsewhere herein.

The “patient” or “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the patient or subject in accordance with the present invention is a human. The patient referred to in accordance with the present invention shall be a patient presenting with suspected acute coronary syndrome (ACS), preferably at the emergency department. Typically, such a patient shall either suffer from ACS or shall exhibit at least one or more symptoms accompanying ACS, such as chest pain. In an embodiment, the subject shall suffer from unstable angina.

The term “acute coronary syndrome (ACS)” as used herein refers to an obstructive event affecting coronary vessels involving multiple interrelated mechanisms. Preferably, in ACS a plaque may rupture or erode, in response to inflammation, leading to local occlusive or non-occlusive thrombosis. Depending on the degree and reversibility of this dynamic obstruction, the clinical manifestations of ACS comprise a continuous spectrum of risk that progresses from unstable angina (UA) to non-ST-segment elevation myocardial infarction (NSTEMI) to ST-segment elevation myocardial infarction (STEMI). NSTEMI is distinguished from UA by ischemia sufficiently severe in intensity and duration to cause myocyte necrosis, which is recognized by the detection of cardiac Troponins, the most sensitive and specific biomarker of myocardial injury. ACS is typically accompanied by prolonged chest pain episodes, preferably, 20 min or longer.

In a preferred embodiment, the patient to be tested is suspected to suffer from non-ST-segment elevation acute coronary syndrome that comprise myocardial infarction (NSTEMI) and unstable angina. Thus, the patient does not suffer from ST-segment elevation myocardial infarction (STEMI). STEMI is defined in the presence of persisting ST segment elevations in at least 2 contiguous leads or a new bundle branch block (right or left bundle branch block) or a permanently paced rhythm. A subject who is suspected to suffer from NSTEMI, preferably, has a normal or non-diagnostic, or ST-segment depressions or T-wave inversions on the ECG and thus, does not have such ST segment elevations.

The term “data” as used herein refers to digital information such as numerical values indicative for the parameters of the set of parameters for which data shall be received in accordance with the present invention. Preferably, the digital numerical values shall represent amounts of compounds to be considered or counts of blood cells or thrombocyte level. Other digital information considered in the method according to the present invention may be identifier, e.g., identifier of gender, identifier for normal or impaired ECG, identifiers for certain events in medical history of a patient such as those mentioned elsewhere in accordance with the method of the present invention or numerical identifier of age.

In an embodiment of the present invention, the methods of the present invention are computer-implemented methods. Typically, all steps of the computer-implemented methods of the present invention are performed by one or more processing units of a computer or a computer network. However, the computer-implemented method may comprise additional steps, such as the determination of the amount of a marker in a sample, such as the amount of a cardiac Troponin in the first and the second sample, or such as the thrombocyte level.

In accordance with the methods of the present invention, as set of parameters shall be assessed, in particular in the “predictive” and the “prognostic methods”. The term “set of parameters” as referred to herein means a collection of different parameters selected from the aforementioned group of different parameters which shall be considered for carrying out the methods of the present invention. Said set of parameters shall comprise at least six, at least seven, preferably, eight, nine or ten parameters in the case of the method of predicting the risk of an adverse event of a patient presenting with suspected acute coronary syndrome or at least eight, preferably, eight, nine, ten, eleven, twelve, thirteen, fourteen or fifteen in the case of the method for predicting the need of myocardial revascularization of a patient presenting with suspected acute coronary syndrome.

In some embodiments, at least five parameters (i.e. of the above parameters) are assessed In some embodiments, at least six parameters are assessed.

In some embodiments, at least seven parameters are assessed.

In some embodiments, at least eight parameters are assessed.

In some embodiments, nine parameters are assessed.

In some embodiments, ten parameters are assessed. Thus, all parameters are assessed.

### “Prognostic” Method

In accordance with the “prognostic” method, at least five, at least six, such as at least seven, eight, nine or ten of following parameters shall be assessed
The amount of a cardiac Troponin in a first sample obtained from said patient at presentation.
The amount of said cardiac Troponin in a second sample obtained from said patient within 30 minutes to 6 hours after the first sample, such as within 1 hour to 3 hours after the first sample, such as within 1 hour to 2 hours after the first sample.
the amount of CRP (C-reactive protein) in a sample from the patient
at least one parameter for the patient's renal function such as the amount of urea in a sample from the patient, the patient's GFR and, in particular the patient's serum creatinine amount.
The amount of sodium in a sample from the patient,
The amount of hemoglobin in a sample from the patient,
The patient's thrombocyte level,
The patient's age
The patient's gender, and
The presence or absence of a normal ECG in said patient.

In some embodiments of the methods described herein, the presence or absence of normal ECG can be assessed based on ECG readings obtained from the subject.

As set forth above, at least five, six or seven of the above-parameters are assessed for the prognostic method. The full model comprise ten parameters. However, it is envisaged that the prognostic method comprises the assessment of at least the following five parameters (out of the ten parameters).
The amount of a cardiac Troponin in a first sample obtained from said patient at presentation.
The amount of said cardiac Troponin in a second sample obtained from said patient within 30 minutes to 6 hours after the first sample, such as within 1 hour to 3 hours after the first sample, such as within 1 hour to 2 hours after the first sample.
At least one parameter for the patient's renal function. Preferably, the parameter is the patient's serum creatinine amount.
The patient's age
The patient's gender.
The presence or absence of a normal ECG in said patient.

In an embodiment, all ten i) to x) parameters are assessed (or a1) to a10).)

### Predictive Method

At least eight, such as at least eight, nine, ten, eleven, twelve, thirteen, fourteen or fifteen of following parameters shall be assessed in accordance with the “predictive” methods:
the amount of a cardiac Troponin in a first sample obtained from said patient at presentation,
the amount of said cardiac Troponin in a second sample obtained from said patient within 30 minutes to 6 hours after the first sample, such as within 1 hour to 3 hours after the first sample, such as within 1 hour to 2 hours after the first sample,
the patient's gender
the patient's age [in years],
the patient's serum creatinine amount
the presence or absence of a normal ECG in said patient
the presence or absence of chest symptoms, in particular chest pain, in said patient
information on the patient's history of smoking
a parameter for the patient's renal function selected from the group consisting of the patient's GFR, the patient's serum creatinine amount, and the patient's blood urea nitrogen (BUN) amount (or urea), in particular the patient's serum creatinine amount
the presence or absence of dyspnea in said patient, and
information on the patient's past medical history, comprising at least one, preferably all, of the following: information on the patient's history of diabetes, and information on the patient's history of history of coronary artery disease (such as information on the patient's history of revascularization).

As set forth above, at least eight, nine, ten, eleven, twelve, thirteen, fourteen or fifteen of the above-parameters are assessed for the predictive method. However, it is envisaged that the predictive method comprises the assessment of the following eight parameters.
the amount of a cardiac Troponin in a first sample obtained from said patient at presentation,
the amount of said cardiac Troponin in a second sample obtained from said patient within 30 minutes to 6 hours after the first sample, such as within 1 hour to 3 hours after the first sample, such as within 1 hour to 2 hours after the first sample,
the patient's gender
the patient's age [in years]
the patient's serum creatinine amount
the presence or absence of chest pain as the leading symptom
information on the patient's history of coronary artery disease, such as on the history of myocardial revascularization (and, thus whether the patient has been subjected a myocardial revascularization in the past)
information on the patient's history of smoking (past or present).

In an embodiment, all eight parameters are assessed.

Creatinine amount in serum is needed to assess the patient's renal function. Alternatively or additionally, the least one parameter for the patient's renal function is the amount of urea. Alternatively, the amount of BUN can be assessed. Moreover, the least one parameter for the patient's renal function is the patient's eGFR or GFR.

In accordance with the predictive and the prognostic method, the amount of a cardiac Troponin and in a first sample and second sample shall be assessed (or information on the amount shall be taken into account). The first sample shall have been obtained at presentation. Moreover, the second sample shall have been obtained from said patient, preferably, within about 30 minutes to about 6 hours after the first sample, more preferably within about 1 hour to about 3 hours after the first sample, and most preferably within about 1 hour to about 2 hours after the first sample. In some embodiments, the second sample has been obtained about 1 hour after the first sample. In some embodiments, the second sample has been obtained about 2 hours after the first sample.

The amount of the cardiac Troponin in the second sample is typically used in order to calculate that the difference between the amount of the cardiac Troponin in the first sample and the amount in the second sample (delta). Thus, the methods of the present invention may encompass the calculation of this difference, such as by the processing unit. The difference can be given as a value. Preferably, the value is used as a parameter for the predictive and prognostic methods as referred to herein.

The data for the set of parameters as specified above are received by a processing unit. Typically, said data may be received from a database comprising stored data for the parameters referred to in accordance with the present invention. Alternatively or in addition the data may be received from measurement equipment performing real-time measurements on samples of the patient. It will be understood that there are parameters which cannot be measured but need to be acquired by other means from the subject and stored into a database. These parameters include, e.g., age, gender, and medical history. The data can be received from the database(s) or real-time measurement equipment via physical connections or wireless data transfer. Thus, such data transmission may be achieved by a permanent or temporary physical connection, such as coaxial, fiber, fiber-optic or twisted-pair, cables. Typically, however, it may be achieved by a temporary or permanent wireless connection using, e.g., radio waves, such as Wi-Fi, LTE, LTE-advanced or Bluetooth.

The processing unit as referred to in accordance with the method of the present invention, typically, comprises a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. Preferably, the data processing unit is a computer or computer-like device such as a tablet, smart device or mobile device.

The data processing unit shall carry out an analysis of the set of parameters, wherein said analysis comprises calculating a score for predicting the risk of an adverse event of said patient based on the set of parameters received in step a). Thus, the processing unit needs software instructions tangibly embedded on said unit which when run on the processing unit carry out the analysis of the parameters including the calculation of the score for predicting the risk of an adverse event of said patient or the score for predicting the need of myocardial revascularization of said patient.

The processing unit shall also provide information on the calculated score such that an adverse event or the need of myocardial revascularization can be predicted. Typically, the score which is calculated in accordance with the method of the present invention will be compared by the processing unit with at least one identifier comprising information for prediction of an adverse event or the need of myocardial revascularization stored in a database. Thus, if the calculated score can be linked by the processing unit to the said identifier, the information linked to said identifier can be linked to the score and the prediction of an adverse event or the need for myocardial revascularization can be provided.

The term “score” as used in accordance with the methods of the present invention, in particular the predictive and prognostic methods, refers to a parameter integrating the information comprised by the aforementioned set of parameters. By using a scoring system as described herein, advantageously, values of different dimensions or units for the parameters may be used, since the values will be mathematically transformed into the score. Preferably, a low score is associated with a low risk, and a high score with a high risk. Typically, a score is a single value which is calculated based on other values by applying mathematical operations which weight such other values according to predetermined rules. Accordingly, some values, such as the amount of cardiac Troponins, may affect the score more than other such as gender or age. Preferably, the score in accordance with the present invention can be calculated as described herein below in detail.

Preferably, the score shall allow for assessing whether a patient is at risk of an adverse event, or not (in the prognostic method) or for assessing whether the subject is in need of myocardial revascularization, or not (in the predictive method). Preferably, the score is calculated based on a suitable scoring algorithm. Said scoring algorithm, preferably, shall allow for the aforementioned assessment, based on the set of parameters. The read-out may be the percent probability for the predicted event.

For example the formula for prognosis of all-cause death within 365 days using the full model estimates plugged into a penalized logistic regression formula:

TABLE 1
Penalized logistic regression formula for prediction
of death at 365 days using full model

| | | | | Odds |
|---|---|---|---|---|
| | Term | Estimate | penalty | Ratio |
| 1 | (Intercept) | −4.139 | 0.024 | 0.016 |
| 2 | ECG sinus rhythm | −0.331 | 0.024 | 0.718 |
| | (binary) | | | |
| 3 | Sodium (numeric) | −0.188 | 0.024 | 0.828 |
| 4 | Hemoglobin | −0.158 | 0.024 | 0.854 |
| | (numeric) | | | |
| 5 | Sex (binary) | −0.089 | 0.024 | 0.915 |
| 6 | Creatinine | −0.008 | 0.024 | 0.992 |
| | (numeric) | | | |
| 7 | Thrombocyte count | 0.064 | 0.024 | 1.066 |
| | (numeric) | | | |
| 8 | Troponin delta | 0.111 | 0.024 | 1.118 |
| | (numeric) | | | |
| 9 | Age (numeric) | 0.253 | 0.024 | 1.288 |
| 10 | Interaction | 0.257 | 0.024 | 1.293 |
| | Age: Troponine_c0 | | | |
| 11 | Troponine_c0 | 0.287 | 0.024 | 1.333 |
| | (numeric) | | | |
| 12 | t0_crp_value | 0.311 | 0.024 | 1.364 |

Displayed is the model formula consisting of intercept, estimates, penalty and odds ratio for each of the included variables, i.e. sodium concentration, ECG, hemoglobin level, sex, creatinine, thrombocyte count, delta troponin, age, C-reactive protein, and initial troponin (from top to bottom). Note that the parameters of the minimal model: delta troponin, creatinine, sex, age and initial troponin are preserved in the full model as they are preserved in all 33 models.

For example, the score for method for assessing the need of revascularization due to obstructive CAD with eight parameters (minimal method, see Examples) the score may be calculated by plugging the following estimates into a penalized logistic regression model:

TABLE 2
Penalized logistic regression formula for prediction of
obstructive CAD requiring myocardial revascularization

| | Term | estimate | penalty | OR |
|---|---|---|---|---|
| 1 | Sex (binary) | −0.741 | 0.019 | 0.476 |
| 2 | Creatinine | 0.049 | 0.019 | 1.05 |
| | (numeric) | | | |
| 3 | Interaction | 0.078 | 0.019 | 1.081 |
| | Age: Troponine_c0 | | | |
| 4 | Age (numeric) | 0.119 | 0.019 | 1.126 |
| 5 | Troponin delta | 0.125 | 0.019 | 1.133 |
| | (numeric) | | | |
| 6 | (Intercept) | 0.128 | 0.019 | 1.137 |
| 7 | Smoking history | 0.332 | 0.019 | 1.393 |
| | (binary) | | | |
| 8 | Troponine_c0 | 0.476 | 0.019 | 1.61 |
| | (numeric) | | | |
| 9 | Chest Pain | 0.726 | 0.019 | 2.066 |
| | (binary) | | | |
| 10 | History of | 0.746 | 0.019 | 2.109 |
| | Revascularization | | | |
| | (binary) | | | |

Displayed is the model formula consisting of intercept, estimates, penalty and odds ratio for each of the included variables, i.e. sodium concentration, ECG, hemoglobin level, sex, creatinine, age, delta troponin, history of smoking, initial troponin, chest pain as leading symptom, history of coronary artery disease, such as the history of myocardial revascularization (from top to bottom). Note that the parameters of the minimal model are preserved in the full model comprising fifteen variables and in all 33 models.

The present invention further relates to computer program including computer-executable instructions for performing the steps of the computer-implemented method according to the present invention, when the program is executed on a computer or computer network. Typically, the computer program specifically may contain computer-executable instructions for performing the steps of the method as disclosed herein. Specifically, the computer program may be stored on a computer-readable data carrier.

The present invention further relates to computer program product with program code means stored on a machine-readable carrier, in order to perform the method according to present invention, when the program is executed on a computer or computer network, such as one or more of the above-mentioned steps discussed in the context of the computer program. As used herein, a computer program product refers to the program as a tradable product. The product may generally exist in an arbitrary format, such as in a paper format, or on a computer-readable data carrier. Specifically, the computer program product may be distributed over a data network.

The present invention further relates to a computer or computer network comprising at least one processing unit, wherein the processing unit is adapted to perform all steps of the method according to the present invention.

The present invention also, in principle, contemplates a computer program, computer program product or computer readable storage medium having tangibly embedded said computer program, wherein the computer program comprises instructions when run on a data processing device or computer carrying out the method of the present invention as specified above. Specifically, the present disclosure further encompasses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising data of parameters as defined herein elsewhere, and
a data stream signal, typically encrypted, comprising the score calculated by the methods of the present invention and, preferably, providing information of the predictions.

The present invention further relates to a device for predicting of the risk of an adverse event or predicting the need of myocardial revascularization, said device comprising a processing unit, and a computer program including computer-executable instructions (such as a computer program as set forth above), wherein said instructions, when executed by the processing unit, causes the processing unit to perform the computer-implemented method according to the present invention, i.e. to perform the steps of said method. The device may further comprise a user interface and a display, wherein the processing unit is coupled to the user interface and the display. Typically, the device provides as output the prediction. In an embodiment, the classification is provided on the display.

All references cited throughout this specification are herewith incorporated by reference with respect to the specifically mentioned disclosure content above as well as in their entireties.

The Figures show:

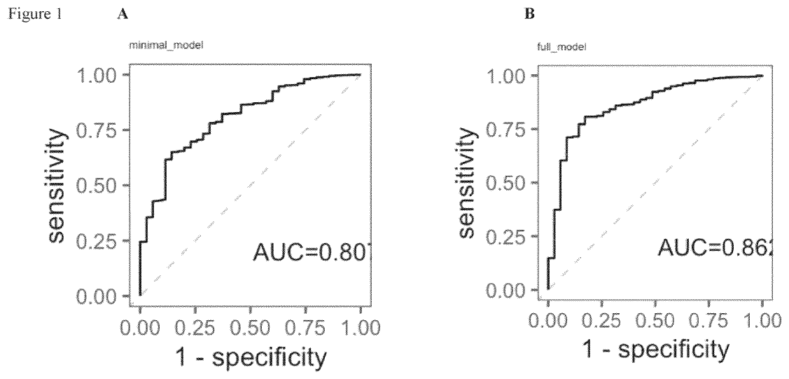

FIG. 1: Discriminatory ability to predict all-cause death at 365 days
A) The minimal model (left panel) demonstrates a moderate discriminatory ability with an AUC of 0.81 for prediction of all-cause death at 365 days. B) The full model shows a good discriminatory ability with an AUC of 0.86 to predict all-cause death at 365 days.

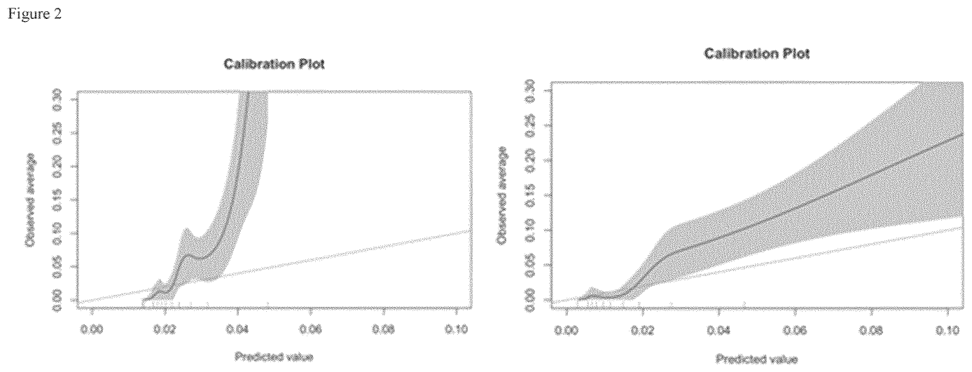

FIG. 2: Calibration plot between estimated and observed all-cause death within 365 days using the minimal model and the full model
The calibration plot displays the agreement between estimated (black line) and observed (grey line) all-cause death using the minimal prognostic model of five variables (left panel) or the full prognostic model containing 10 parameters. The agreement is linear for the full model which systematically overestimates the observed event. The agreement for the minimal model is linear over a narrow range from 0.5 to 3.5% and subsequently an exponential rise.

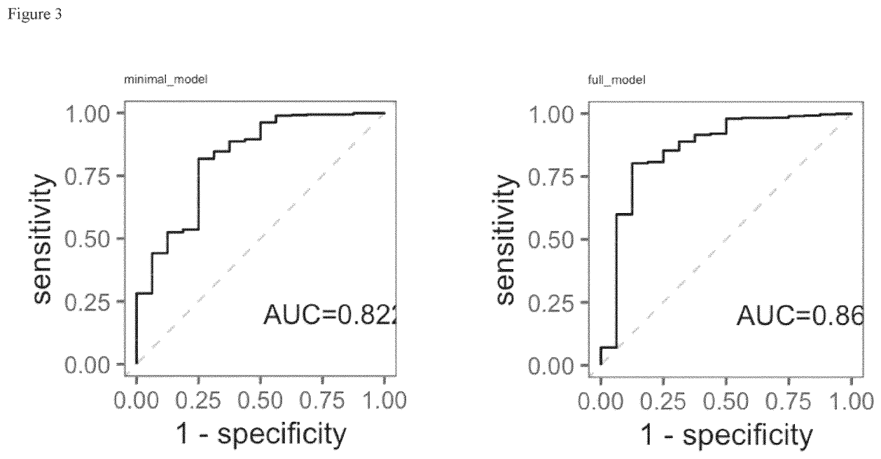

FIG. 3: Discriminatory ability to predict all-cause death at 180 days
The minimal model (left panel) demonstrates a moderate discriminatory ability with an AUC of 0.82 for prediction of all-cause death at 180 days. The full model shows an excellent discriminatory ability with an AUC of 0.86 to predict all-cause death at 160 days.

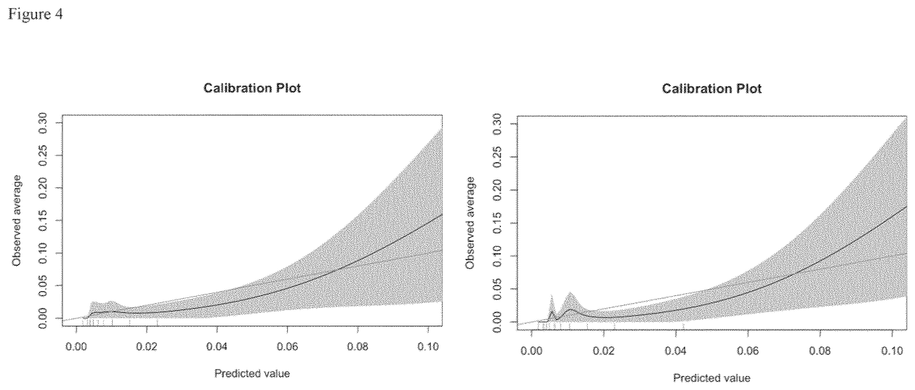

FIG. 4: Calibration plot between estimated and observed all-cause death within 180 days using the minimal model and the full model
The calibration plot displays the agreement between estimated (black line) and observed (grey line) all-cause death using the minimal prognostic model of five variables (left panel) or the full prognostic model containing 10 parameters.

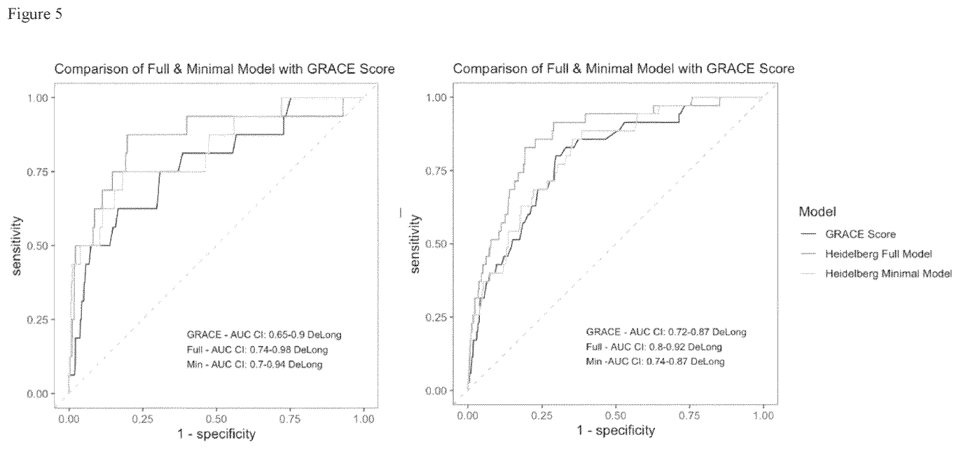

FIG. 5: Performance of GRACE score and comparison with full and minimal models at 180 days and 365 days.
Comparison of GRACE score (black line), minimal model (light grey line), and full model (dark grey line) for prediction of death at 180 days (left panel) and prediction of death at 365 days (right panel.
At 365 days (right panel), the full model outperforms the GRACE score with a statistically significant (p=0.02) gain of AUC (=delta AUC) of 0.07. Hence, the full model proved better prognostic performance than the state-of-the-art comparator. At 180 days (left panel), there is a trend for a significant gain of prognostic performance of the full model versus the GRACE score (p=0.085).

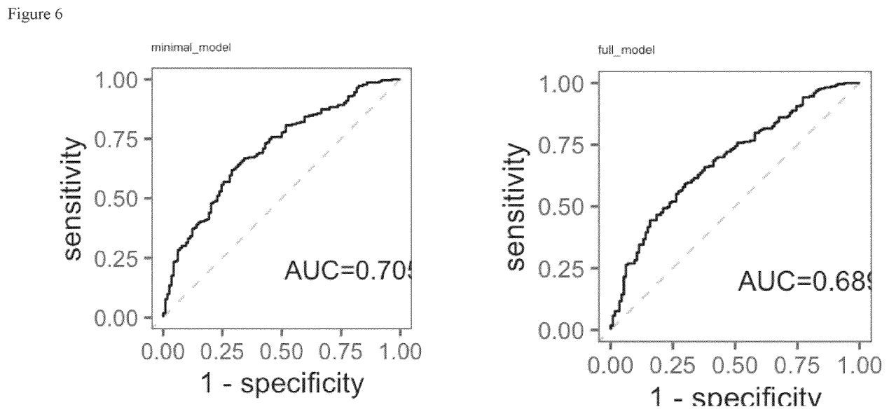

FIG. 6: Performance of full and minimal model to predict obstructive CAD requiring revascularization.
Receiver operating curve (ROC) showing an AUC of 0.71 for the minimal model (left panel) and AUC of 0.69 for the full model (right panel).

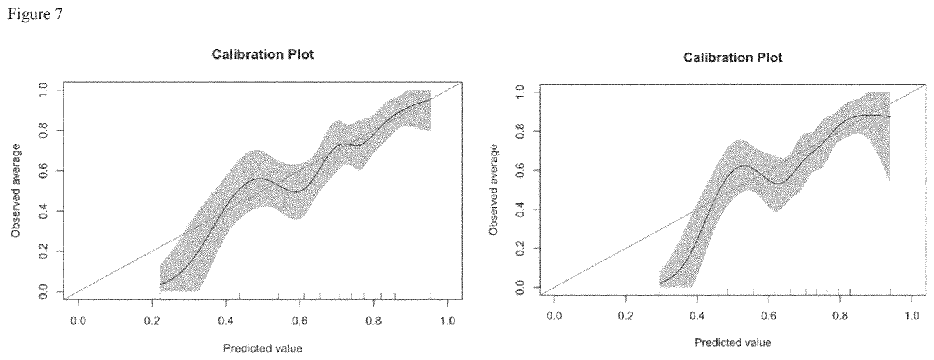

FIG. 7: Calibration plots of minimal and full model for prediction of revascularization within 30 days.
Calibration plot for the minimal model (left panel) and for the full model (right panel) showing almost linear correlation between predicted (dark line) and observed (grey line) output values.

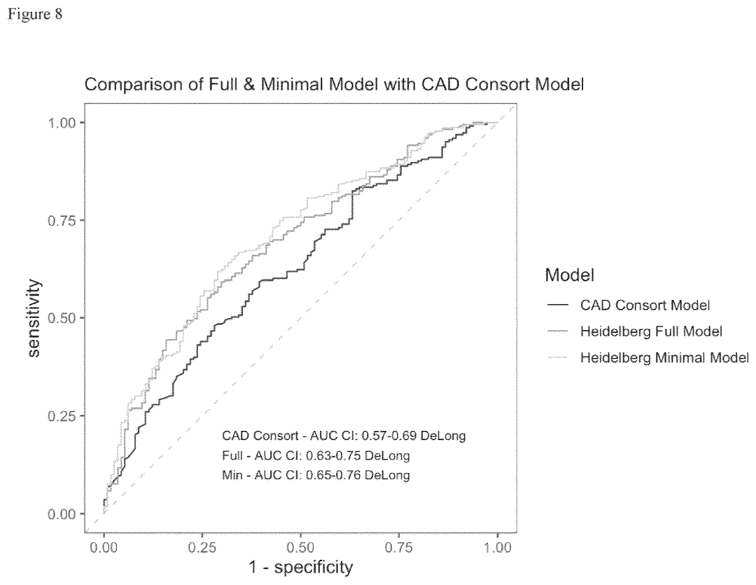

FIG. 8: Performance of ESC Consortium algorithm and comparison with full and minimal models
The figure shows the AUC of the ESC consortium algorithm that comprise 9 variables. The overall performance is moderate with an AUC of 0.63 (95% CI: 0.65-0.76). The AUC of the minimal model is 0.71 (95% CI: 0.65-0.76), and the AUC of the full model is 0.69 (95% CI: 0.63-0.75). The minimal model outperforms the ESC Consortium algorithm significantly (p=0.004) with a gain in AUC of 0.08. The full model also outperforms the ESC consortium algorithm significantly (p=0.04) with a gain in AUC of 0.06.

### EXAMPLES

The Examples shall illustrate the invention. They shall be no means construed as limiting the scope.

### Example 1: Baseline Characteristics of Derivation Cohort (Training Set) and Validation Cohort (Test Set)

Patients presenting with symptoms suggestive of myocardial infarction in which serial 5 high-sensitivity cardiac troponin T measurements were obtained at presentation and later within the emergency department were included. Patients with persisting ST-segment elevation myocardial infarction (STEMI) or those with a presumably new left bundle branch block, and those deemed ineligible as indicated earlier were excluded. Both derivation and validation cohorts were prospective, included serial hs-cTnT concentrations, and the final diagnosis of NSTEMI was adjudicated according to the Universal Definition of Myocardial Infarction. Diagnosis of NSTEMI required an elevation of hs-cTnT above a prespecified cutoff and a respective relevant rise and/or fall, together with clinical signs or symptoms suggesting an ischemic context. These signs or symptoms included ischemic symptoms (but not exclusively typical chest pain), new or presumed new significant ST-T wave changes except ST segment elevations, development of pathological Q waves, imaging evidence of new loss of viable myocardium or new regional wall motion abnormality, and/or identification of an intracoronary thrombus by angiography or autopsy. The algorithm was derived from patients recruited in Germany, at the Emergency Department of the Heidelberg University Hospital from Jul. 1, 2016 to Jul. 1, 2018. The cohort collected follow-up data for 12 months until 1 Jul. 2019.

The entire cohort comprised 3,928 patients of whom 3018 were classified as rule-out and 910 were triaged as observe zone. Within 180 days and 365 days, all-cause death occurred in 65 (1.65%) and 100 (1.82%) patients, respectively. Briefly, the study cohort was predominantly male (55.91%, n=2,195) with a mean age of 60.7 years. The overall discharge rate was 70.1% (2,801 of 3,928 patients) including mostly patients (2,343, of 2,801 patients) classified into the rule-out zone (83.6%).

Rates of coronary angiography within 30 days of index event were 1,344 out of 4,934 eligible patients (27.2%) of whom 889 patients required revascularization (66.1%), respectively.

The parameters for the models were derived and trained in 75% of the entire study cohort and the models were subsequently tested in 25% of the entire study cohort. The random selection of patients was stratified for outcome events was ensured to eliminate bias.

Rates of all-cause death at 30 and 90 days were 29 patients (0.74%) and 52 patients (1.3%), respectively. Rates of all-cause death at 180 days and 365 days were 65 patients (1.7%) and 100 patients (2.5%), respectively. Among 1,344 patients with coronary angiography within 30 days from index event a total of 889 patients (66.1%) required myocardial revascularization for obstructive coronary artery disease.

### Example 2: Algorithm Development

The logistic regression with elastic for a binary outcome was applied to identify predictors and contruct predictive and prognostic models in a training set that comprised 75% of the entire study population. The model was trained using 5-fold cross validation. A total of 33 models was developed by permutating a set of defined variables—After model training performance was measured in a blinded test set comprising 25% of the study population. All estimated parameters were collected strictly on the training data set only. Since we used mean imputation for numeric variables and mode imputation for nominal variables, mean and mode imputation values were trained on the training data set only. Skewed variables were log transformed and all numeric variables were centered and scaled based on the distribution of the training data. Further, we considered the interaction term between patient's baseline Troponin and patient's age as relevant and added it to the model. Using 5-fold cross validation we estimated the hyperparameters penalty (represents the amount of penalty applied to logistic regression model) and mixture (represents the relative amount of penalties L1 and L2) needed for elastic net regularization. The hyperparameter grid consisted of 1,000 parameter combinations each of which was fitted in the model selection process during the 5-fold cross validation. The best fitted hyperparameters were evaluated based on performance of ROC-AUC on the 5-fold cross validation. After finding the best hyperparameters we finally fit the model on the whole training data set to obtain estimates of the covariates. We chose ROC-AUC to be our primary performance measure. We then calculated the Youden-Index on the ROC curve to obtain a threshold for reporting sensitivity and specificity of our models. Additionally, algorithm calibration was assessed on the validation cohort (test set) by plotting calibration curves of predicted values and the observed average. The ML algorithm allowed estimating the individual risk of each patient attributing a probability for the adverse event within the pre-specified follow-up interval.

### Example 3: Algorithm for the Prediction of Mortality

### 3.1 Follow Up Completeness and Duration

For the outcome variable in the predictive models, all-cause death (all-cause mortality) was chosen according to the Academic Research Consortium-2 Consensus Document (European Heart Journal 2018, 2192-220). Patients were followed up for all-cause mortality for a median of 468 (374-670) days, and follow-up was complete for 99.0% (missing follow-up in 41 of 3,969 cases). We analyzed death as a binary outcome (180- and 365-days all-cause death). Complete information on vital status at 90 days at 180 days and 365 days was retrieved in all but 41 patients (99.0%).

### 3.2 Model Selection

Step 1. Correlation and Discrimination within the Derivation Cohort.

The ML algorithm was calibrated by comparing predicted (fitted) probability for the blinded test population versus the actual average values regarding the endpoint all-cause death at 180 and 365 days. The calibration plot for predicted death at 365 days using the full model as well as the minimal and the full model to predict death at 180 days found nearly linear relationship between predicted and actual mortality in the independent test population, particularly within the mortality range of interest from 0 to 3% (FIG. 1). Overall, predicted risk of mortality slightly overestimated the actual mortality. This reduces the risk of unwarranted discharge and is preferred over an underestimation of mortality risk that would lead to unsafe discharge and undertreatment.

For model selection, following points were taken into consideration:

### 1) Patient Population

### Rules Applied:

From clinical perspective, patients classified as rule in by a validated hs-cTn protocol have a very high risk of myocardial infarction and require immediate diagnostic and therapeutic measures. Therefore, rule-in patients were excluded from this model. Only patients that initially classified into the observation zone or as rule out for myocardial infarction were considered. 3,018 patients were classified into rule-out and 910 patients were classified into the observe zone (rule in n=965 excluded), resulting in a sample size of n=3,969 patients. In 41 patients the outcome of interest was missing. Therefore, the final sample size consisted of n=3,928 patients.

### 2) Outcome Variables Timing and Outcome Measure

### Outcome:

Follow up for death was available. In the study population of rule out and observation zone, the event rate of all-cause death within 180 and within 365 days were tested.

### Timing of Events:

Given follow up length of the populations of at least 12 months, timing of the binary outcome was considered to be feasible between 180 days and 365 day<s. In this population with suspected ACS, event rates at 1 month (0.5%) and 90 days (1.1%) were too low to create a predictive model with good properties. The full model to predict death at 365 days performed better than the GRACE score, with a significant gain of discriminatory ability (p=0.02). The full model and other models based on events at 6 months performed similarly well albeit statistically not significantly better than the GRACE score, presumably due to a clinical course not directly connected with current presentation and not strongly correlating with predictor variables at baseline, and due to smaller numbers of events.
A 180 to 365 days mortality outcome is clinically reasonable and of substantial relevance regarding further clinical workup and decisions. A model using 180 to 365 days mortality also yielded good diagnostic properties and was well calibrated in the region of interest (0-3%).

### 3) Predictors Variables: Demographic, Clinical, Vital Signs, ECG and Laboratory

Different models were tested. The best performing model was well calibrated and had a good diagnostic performance in the blinded test cohort with an AUC 0.86 (95% confidence interval 0.80-0.92). It consisted of rule out and observation zone patients only that were randomly classified to a 25% test set (n=982). Predictor variables were age, sex, ECG parameters, and laboratory values (including hs-cTnT and hs-cTnT kinetics, creatinine, sodium, C-reactive protein, hemoglobin, platelet count).

Tables 3 and 4 show the individual model estimates and the respective parameter with its corresponding relative weight (for 180 and 365 days). The minimal model contains 5 different parameters and the full model contains 10 parameters. Another 31 models are listed as they appear in the table from top to bottom:

TABLE 3
Overview on model equations for prediction of death within 365 days showing
all 33 models ranging from the minimal model to the full model.

| Model | (Intercept) | ΔTrop | Creatinine | Gender | Age: Trop | Age | Trop | ECG | Na | Hb | Thrombo | CRP | Penalty |
|---|---|---|---|---|---|---|---|---|---|---|---|---|---|
| Min | −3.829 | 0 | 0 | 0 | 0.036 | 0.069 | 0.123 | | | | | | 0.061 |
| Full | −4.139 | 0.111 | −0.008 | −0.089 | 0.257 | 0.253 | 0.287 | −0.331 | −0.188 | −0.158 | 0.064 | 0.311 | 0.024 |
| 3 | −3.867 | 0 | 0 | 0 | 0.058 | 0.094 | 0.213 | 0 | | | | | 0.031 |
| 4 | −4.674 | 0.189 | −0.113 | −0.135 | 0.366 | 0.453 | 0.484 | | | | | 0.555 | 0.005 |
| 5 | −3.925 | 0 | 0 | 0 | 0.12 | 0.135 | 0.181 | | −0.12 | | | | 0.061 |
| 6 | −4.384 | 0.147 | −0.078 | −0.136 | 0.312 | 0.367 | 0.474 | | | −0.274 | | | 0.01 |
| 7 | −4.401 | 0.132 | 0 | 0 | 0.334 | 0.384 | 0.51 | | | | 0.178 | | 0.01 |
| 8 | −4.346 | 0.172 | −0.086 | −0.085 | 0.346 | 0.386 | 0.416 | −0.437 | | | | 0.508 | 0.008 |
| 9 | −3.826 | 0 | 0 | 0 | 0.083 | 0.1 | 0.138 | −0.082 | −0.086 | | | | 0.077 |
| 10 | −4.149 | 0.15 | −0.103 | −0.139 | 0.313 | 0.362 | 0.464 | −0.491 | | −0.276 | | | 0.008 |
| 11 | −4.108 | 0.143 | −0.014 | −0.052 | 0.336 | 0.364 | 0.451 | −0.463 | | | 0.187 | | 0.012 |
| 12 | −4.016 | 0.012 | 0 | 0 | 0.15 | 0.159 | 0.203 | | −0.13 | | | 0.215 | 0.049 |
| 13 | −4.556 | 0.169 | −0.104 | −0.203 | 0.329 | 0.39 | 0.418 | | | −0.201 | | 0.488 | 0.008 |
| 14 | −4.621 | 0.182 | −0.083 | −0.177 | 0.368 | 0.44 | 0.472 | | | | 0.132 | 0.513 | 0.006 |
| 15 | −4.45 | 0.141 | −0.067 | −0.147 | 0.327 | 0.349 | 0.449 | | −0.293 | −0.238 | | | 0.01 |
| 16 | −3.925 | 0 | 0 | 0 | 0.12 | 0.135 | 0.181 | | −0.12 | | 0 | | 0.061 |
| 17 | −4.408 | 0.152 | −0.077 | −0.225 | 0.331 | 0.403 | 0.506 | | | −0.252 | 0.183 | | 0.008 |
| 18 | −4.038 | 0.051 | 0 | 0 | 0.211 | 0.209 | 0.258 | −0.229 | −0.167 | | | 0.282 | 0.031 |
| 19 | −4.28 | 0.159 | −0.09 | −0.163 | 0.313 | 0.346 | 0.37 | −0.417 | | −0.196 | | 0.458 | 0.01 |
| 20 | −3.865 | 0 | 0 | 0 | 0.113 | 0.129 | 0.166 | −0.12 | | | 0 | 0.185 | 0.061 |
| 21 | −4.018 | 0.096 | 0.007 | −0.042 | 0.238 | 0.234 | 0.287 | −0.331 | −0.223 | −0.188 | | | 0.031 |
| 22 | −4.053 | 0.11 | 0.021 | −0.019 | 0.283 | 0.278 | 0.34 | −0.367 | −0.243 | | 0.114 | | 0.024 |
| 23 | −4.143 | 0.15 | −0.083 | −0.208 | 0.327 | 0.379 | 0.466 | −0.486 | | −0.247 | 0.181 | | 0.008 |
| 24 | −4.331 | 0.113 | −0.008 | −0.081 | 0.26 | 0.262 | 0.302 | | −0.199 | −0.168 | | 0.323 | 0.024 |
| 25 | −3.948 | 0 | 0 | 0 | 0.115 | 0.129 | 0.168 | | −0.105 | | 0 | 0.178 | 0.061 |
| 26 | −4.548 | 0.169 | −0.091 | −0.239 | 0.337 | 0.397 | 0.424 | | | −0.184 | 0.105 | 0.471 | 0.008 |
| 27 | −4.398 | 0.133 | −0.038 | −0.166 | 0.322 | 0.339 | 0.427 | | −0.269 | −0.213 | 0.123 | | 0.012 |
| 28 | −4.243 | 0.132 | −0.046 | −0.118 | 0.294 | 0.293 | 0.327 | −0.365 | −0.206 | −0.171 | | 0.365 | 0.015 |
| 29 | −3.878 | 0 | 0 | 0 | 0.113 | 0.125 | 0.163 | −0.116 | −0.103 | | 0 | 0.176 | 0.061 |
| 30 | −4.098 | 0.117 | −0.01 | −0.088 | 0.253 | 0.261 | 0.294 | −0.347 | | −0.171 | 0.082 | 0.342 | 0.024 |
| 31 | −3.974 | 0.086 | 0.024 | −0.045 | 0.218 | 0.216 | 0.262 | −0.304 | −0.2 | −0.169 | 0.078 | | 0.039 |
| 32 | −3.956 | 0 | 0 | 0 | 0.109 | 0.122 | 0.161 | | −0.101 | −0.086 | 0 | 0.172 | 0.061 |
| 33 | −4.139 | 0.111 | −0.008 | −0.089 | 0.257 | 0.253 | 0.287 | −0.331 | −0.188 | −0.158 | 0.064 | 0.311 | 0.024 |

minimal_model
full model
minimal_model + ekg_sinus_normal
minimal_model + t0_na_value
minimal_model + t0_hb_value
minimal_model + t0_thrombo_value
minimal_model + ekg_sinus_normal + t0_crp_value
minimal_model + ekg_sinus_normal + t0_na_value
minimal_model + ekg_sinus_normal + t0_hb_value
minimal_model + ekg_sinus_normal + t0_thrombo_value
minimal_model + t0_crp_value + t0_na_value
minimal_model + t0_crp_value + t0_hb_value
minimal_model + t0_crp_value + t0_thrombo_value
minimal_model + t0_na_value + t0_hb_value
minimal_model + t0_na_value + t0_thrombo_value
minimal_model + t0_hb_value + t0_thrombo_value
minimal_model + ekg_sinus_normal + t0_crp_value + t0_na_value
minimal_model + ekg_sinus_normal + t0_crp_value + t0_hb_value
minimal_model + ekg_sinus_normal + t0_crp_value + t0_thrombo_value
minimal_model + ekg_sinus_normal + t0_na_value + t0_hb_value
minimal_model + ekg_sinus_normal + t0_na_value + t0_thrombo_value
minimal_model + ekg_sinus_normal + t0_hb_value + t0_thrombo_value
minimal_model + t0_crp_value + t0_na_value + t0_hb_value
minimal_model + t0_crp_value + t0_na_value + t0_thrombo_value
minimal_model + t0_crp_value + t0_hb_value + t0_thrombo_value
minimal_model + t0_na_value + t0_hb_value + t0_thrombo_value
minimal_model + ekg_sinus_normal + t0_crp_value + t0_na_value + t0_hb_value
minimal_model + ekg_sinus_normal + t0_crp_value + t0_na_value + t0_thrombo_value
minimal_model + ekg_sinus_normal + t0_crp_value + t0_hb_value + t0_thrombo_value
minimal_model + ekg_sinus_normal + t0_na_value + t0_hb_value + t0_thrombo_value
minimal_model + t0_crp_value + t0_na_value + t0_hb_value + t0_thrombo_value
minimal_model + ekg_sinus_normal + t0_crp_value + t0_na_value + t0_hb_value + t0_thrombo_value
if an box is empty the parameter is not included in the model

TABLE 4
Overview on model equations for prediction of death within 180 days showing
all 33 models ranging from the minimal model to the full model.

| Model | (Intercept) | Creatinine | ΔTrop | Age: Trop | Gender | Age | Trop | Hb | Na | Thrombo | ECG | CRP | Penalty |
|---|---|---|---|---|---|---|---|---|---|---|---|---|---|
| Min | −4.994 | −0.084 | 0.099 | 0.314 | 0.353 | 0.503 | 0.62 | | | | | | 0.003 |
| Full | −4.334 | 0 | 0 | 0.116 | 0 | 0.142 | 0.163 | −0.179 | −0.17 | 0 | 0 | 0.227 | 0.039 |
| 3 | −4.848 | −0.076 | 0.096 | 0.317 | 0.347 | 0.475 | 0.575 | | | | −0.19 | | 0.004 |
| 4 | −5.05 | −0.076 | 0.115 | 0.31 | 0.244 | 0.425 | 0.415 | | | | | 0.581 | 0.006 |
| 5 | −4.607 | 0 | 0.027 | 0.262 | 0.117 | 0.292 | 0.346 | | −0.326 | | | | 0.015 |
| 6 | −4.756 | −0.079 | 0.078 | 0.259 | 0.101 | 0.356 | 0.406 | −0.401 | | | | | 0.01 |
| 7 | −5.094 | −0.112 | 0.102 | 0.273 | 0.352 | 0.577 | 0.728 | | | 0.044 | | | 0.001 |
| 8 | −4.274 | 0 | 0 | 0.131 | 0 | 0.165 | 0.185 | | | | −0.013 | 0.264 | 0.039 |
| 9 | −4.403 | 0 | 0 | 0.198 | 0 | 0.231 | 0.283 | | −0.28 | | 0 | | 0.019 |
| 10 | −4.914 | −0.205 | 0.096 | 0.242 | 0.054 | 0.497 | 0.559 | −0.491 | | | −0.147 | | 0 |
| 11 | −4.147 | 0 | 0 | 0.071 | 0 | 0.105 | 0.129 | | | 0 | 0 | | 0.061 |
| 12 | −4.977 | −0.042 | 0.091 | 0.311 | 0.206 | 0.353 | 0.363 | | −0.287 | | | 0.452 | 0.01 |
| 13 | −4.767 | −0.045 | 0.08 | 0.234 | 0.098 | 0.292 | 0.297 | −0.296 | | | | 0.421 | 0.015 |
| 14 | −4.282 | 0 | 0 | 0.131 | 0 | 0.166 | 0.185 | | | 0 | | 0.264 | 0.039 |
| 15 | −5.082 | −0.155 | 0.081 | 0.292 | 0.048 | 0.441 | 0.5 | −0.427 | −0.37 | | | | 0.002 |
| 16 | −4.607 | 0 | 0.027 | 0.262 | 0.117 | 0.292 | 0.346 | | −0.326 | 0 | | | 0.015 |
| 17 | −4.92 | −0.16 | 0.09 | 0.26 | 0.076 | 0.445 | 0.508 | −0.466 | | −0.012 | | | 0.004 |
| 18 | −4.381 | 0 | 0 | 0.163 | 0.002 | 0.188 | 0.208 | | −0.205 | | −0.019 | 0.281 | 0.031 |
| 19 | −4.827 | −0.084 | 0.095 | 0.255 | 0.092 | 0.337 | 0.325 | −0.321 | | | −0.132 | 0.475 | 0.01 |
| 20 | −4.274 | 0 | 0 | 0.131 | 0 | 0.165 | 0.185 | | | 0 | −0.013 | 0.264 | 0.039 |
| 21 | −5.001 | −0.144 | 0.079 | 0.292 | 0.06 | 0.421 | 0.475 | −0.42 | −0.366 | | −0.092 | | 0.003 |
| 22 | −4.403 | 0 | 0 | 0.198 | 0 | 0.231 | 0.283 | | −0.28 | 0 | 0 | | 0.019 |
| 23 | −4.912 | −0.205 | 0.097 | 0.242 | 0.063 | 0.492 | 0.554 | −0.493 | | −0.02 | −0.15 | | 0.002 |
| 24 | −4.746 | −0.021 | 0.063 | 0.228 | 0.09 | 0.254 | 0.268 | −0.262 | −0.245 | | | 0.341 | 0.019 |
| 25 | −4.492 | 0 | 0.005 | 0.194 | 0.032 | 0.22 | 0.238 | | −0.227 | 0 | | 0.319 | 0.024 |
| 26 | −4.621 | −0.014 | 0.064 | 0.204 | 0.102 | 0.242 | 0.254 | −0.265 | | −0.03 | | 0.358 | 0.024 |
| 27 | −4.915 | −0.087 | 0.07 | 0.279 | 0.108 | 0.347 | 0.398 | −0.386 | −0.358 | −0.069 | | | 0.008 |
| 28 | −4.901 | −0.072 | 0.081 | 0.273 | 0.081 | 0.321 | 0.319 | −0.297 | −0.264 | | −0.097 | 0.407 | 0.01 |
| 29 | −4.381 | 0 | 0 | 0.163 | 0.002 | 0.188 | 0.208 | | −0.205 | 0 | −0.019 | 0.281 | 0.031 |
| 30 | −4.305 | 0 | 0 | 0.115 | 0 | 0.149 | 0.169 | −0.188 | | 0 | −0.003 | 0.247 | 0.039 |
| 31 | −4.285 | 0 | 0 | 0.122 | 0 | 0.15 | 0.182 | −0.2 | −0.196 | 0 | −0.011 | | 0.039 |
| 32 | −4.675 | −0.011 | 0.057 | 0.208 | 0.1 | 0.23 | 0.245 | −0.25 | −0.24 | −0.054 | | 0.32 | 0.024 |
| 33 | −4.334 | 0 | 0 | 0.116 | 0 | 0.142 | 0.163 | −0.179 | −0.17 | 0 | 0 | 0.227 | 0.039 |

Table legend: see legend of Table 3 (above).

### Step 2. Performance of the Models on the Test Set

Table legend: This table lists the typical performance measures for prediction of death at 365 days using all 33 models. The list comprise AUC, accuracy, sensitivity, specificity, positive predictive value (PPV), negative predictive value (NPV), precision, recall and f1-Score. The models are sorted the same way as described in the previous table legends.

For example, the performance of the full model for death within 365-days was associated with an AUC of 0.86 (0.80-0.92), a sensitivity of 83, a specificity of 81%, a PPV of 14%, and a NPV of 99%.

TABLE 5
Overview on performance of models for prediction of death within 365 days
showing all 33 models ranging from the minimal model to the full model.

| model | AUC | accuracy | sensitivity | specificity | ppv | npv | precision | recall | f1.score |
|---|---|---|---|---|---|---|---|---|---|
| min | 0.81 | 0.96 | 0.86 | 0.65 | 0.08 | 0.99 | 0.08 | 0.86 | 0.15 |
| full | 0.86 | 0.96 | 0.83 | 0.81 | 0.14 | 0.99 | 0.14 | 0.83 | 0.24 |
| 3 | 0.81 | 0.96 | 0.86 | 0.65 | 0.08 | 0.99 | 0.08 | 0.86 | 0.15 |
| 4 | 0.84 | 0.96 | 0.66 | 0.89 | 0.18 | 0.99 | 0.18 | 0.66 | 0.29 |
| 5 | 0.81 | 0.96 | 0.77 | 0.76 | 0.11 | 0.99 | 0.11 | 0.77 | 0.19 |
| 6 | 0.81 | 0.96 | 0.83 | 0.71 | 0.09 | 0.99 | 0.09 | 0.83 | 0.17 |
| 7 | 0.76 | 0.96 | 0.66 | 0.76 | 0.09 | 0.98 | 0.09 | 0.66 | 0.16 |
| 8 | 0.85 | 0.96 | 0.71 | 0.86 | 0.16 | 0.99 | 0.16 | 0.71 | 0.26 |
| 9 | 0.83 | 0.96 | 0.83 | 0.72 | 0.1 | 0.99 | 0.1 | 0.83 | 0.17 |
| 10 | 0.84 | 0.96 | 0.86 | 0.7 | 0.1 | 0.99 | 0.1 | 0.86 | 0.17 |
| 11 | 0.77 | 0.96 | 0.8 | 0.64 | 0.08 | 0.99 | 0.08 | 0.8 | 0.14 |
| 12 | 0.86 | 0.96 | 0.91 | 0.69 | 0.1 | 1 | 0.1 | 0.91 | 0.18 |
| 13 | 0.85 | 0.96 | 0.71 | 0.85 | 0.15 | 0.99 | 0.15 | 0.71 | 0.25 |
| 14 | 0.82 | 0.97 | 0.71 | 0.82 | 0.13 | 0.99 | 0.13 | 0.71 | 0.22 |
| 15 | 0.82 | 0.96 | 0.77 | 0.8 | 0.12 | 0.99 | 0.12 | 0.77 | 0.21 |
| 16 | 0.81 | 0.96 | 0.77 | 0.76 | 0.11 | 0.99 | 0.11 | 0.77 | 0.19 |
| 17 | 0.79 | 0.96 | 0.89 | 0.62 | 0.08 | 0.99 | 0.08 | 0.89 | 0.15 |
| 18 | 0.86 | 0.96 | 0.89 | 0.74 | 0.11 | 0.99 | 0.11 | 0.89 | 0.2 |
| 19 | 0.86 | 0.96 | 0.94 | 0.66 | 0.09 | 1 | 0.09 | 0.94 | 0.17 |
| 20 | 0.86 | 0.96 | 0.66 | 0.91 | 0.22 | 0.99 | 0.22 | 0.66 | 0.33 |
| 21 | 0.84 | 0.96 | 0.8 | 0.8 | 0.13 | 0.99 | 0.13 | 0.8 | 0.22 |
| 22 | 0.8 | 0.96 | 0.77 | 0.71 | 0.09 | 0.99 | 0.09 | 0.77 | 0.16 |
| 23 | 0.81 | 0.96 | 0.8 | 0.72 | 0.1 | 0.99 | 0.1 | 0.8 | 0.17 |
| 24 | 0.86 | 0.96 | 0.89 | 0.73 | 0.11 | 0.99 | 0.11 | 0.89 | 0.2 |
| 25 | 0.86 | 0.96 | 0.91 | 0.69 | 0.1 | 1 | 0.1 | 0.91 | 0.18 |
| 26 | 0.84 | 0.96 | 0.71 | 0.85 | 0.15 | 0.99 | 0.15 | 0.71 | 0.25 |
| 27 | 0.81 | 0.96 | 0.8 | 0.77 | 0.12 | 0.99 | 0.12 | 0.8 | 0.2 |
| 28 | 0.87 | 0.96 | 0.91 | 0.73 | 0.11 | 1 | 0.11 | 0.91 | 0.2 |
| 29 | 0.87 | 0.96 | 0.86 | 0.77 | 0.12 | 0.99 | 0.12 | 0.86 | 0.21 |
| 30 | 0.86 | 0.96 | 0.71 | 0.87 | 0.17 | 0.99 | 0.17 | 0.71 | 0.27 |
| 31 | 0.83 | 0.96 | 0.89 | 0.69 | 0.09 | 0.99 | 0.09 | 0.89 | 0.17 |
| 32 | 0.87 | 0.96 | 0.86 | 0.8 | 0.13 | 0.99 | 0.13 | 0.86 | 0.23 |
| 33 | 0.86 | 0.96 | 0.83 | 0.81 | 0.14 | 0.99 | 0.14 | 0.83 | 0.24 |

TABLE 6
Overview on performance of models for prediction of death within 180 days
showing all 33 models ranging from the minimal model to the full model.

| model | AUC | accuracy | sensitivity | specificity | ppv | npv | precision | recall | f1.score |
|---|---|---|---|---|---|---|---|---|---|
| min | 0.82 | 0.98 | 0.75 | 0.82 | 0.06 | 0.99 | 0.06 | 0.75 | 0.12 |
| full | 0.86 | 0.98 | 0.88 | 0.8 | 0.07 | 1 | 0.07 | 0.88 | 0.13 |
| 3 | 0.84 | 0.98 | 0.75 | 0.83 | 0.07 | 1 | 0.07 | 0.75 | 0.12 |
| 4 | 0.87 | 0.98 | 0.88 | 0.75 | 0.05 | 1 | 0.05 | 0.88 | 0.1 |
| 5 | 0.81 | 0.98 | 0.69 | 0.86 | 0.07 | 0.99 | 0.07 | 0.69 | 0.13 |
| 6 | 0.83 | 0.98 | 0.69 | 0.86 | 0.08 | 0.99 | 0.08 | 0.69 | 0.14 |
| 7 | 0.82 | 0.98 | 0.75 | 0.83 | 0.07 | 1 | 0.07 | 0.75 | 0.12 |
| 8 | 0.86 | 0.98 | 0.88 | 0.74 | 0.05 | 1 | 0.05 | 0.88 | 0.1 |
| 9 | 0.8 | 0.98 | 0.69 | 0.86 | 0.07 | 0.99 | 0.07 | 0.69 | 0.13 |
| 10 | 0.83 | 0.98 | 0.75 | 0.8 | 0.06 | 0.99 | 0.06 | 0.75 | 0.11 |
| 11 | 0.8 | 0.98 | 0.62 | 0.91 | 0.11 | 0.99 | 0.11 | 0.62 | 0.18 |
| 12 | 0.86 | 0.98 | 0.88 | 0.77 | 0.06 | 1 | 0.06 | 0.88 | 0.11 |
| 13 | 0.86 | 0.98 | 0.81 | 0.85 | 0.08 | 1 | 0.08 | 0.81 | 0.15 |
| 14 | 0.86 | 0.98 | 0.88 | 0.74 | 0.05 | 1 | 0.05 | 0.88 | 0.1 |
| 15 | 0.83 | 0.98 | 0.75 | 0.84 | 0.07 | 1 | 0.07 | 0.75 | 0.13 |
| 16 | 0.81 | 0.98 | 0.69 | 0.86 | 0.07 | 0.99 | 0.07 | 0.69 | 0.13 |
| 17 | 0.83 | 0.98 | 0.69 | 0.86 | 0.07 | 0.99 | 0.07 | 0.69 | 0.13 |
| 18 | 0.86 | 0.98 | 0.88 | 0.76 | 0.06 | 1 | 0.06 | 0.88 | 0.11 |
| 19 | 0.87 | 0.98 | 0.81 | 0.84 | 0.08 | 1 | 0.08 | 0.81 | 0.15 |
| 20 | 0.86 | 0.98 | 0.88 | 0.74 | 0.05 | 1 | 0.05 | 0.88 | 0.1 |
| 21 | 0.83 | 0.98 | 0.75 | 0.84 | 0.07 | 1 | 0.07 | 0.75 | 0.13 |
| 22 | 0.8 | 0.98 | 0.69 | 0.86 | 0.07 | 0.99 | 0.07 | 0.69 | 0.13 |
| 23 | 0.83 | 0.98 | 0.75 | 0.8 | 0.06 | 0.99 | 0.06 | 0.75 | 0.11 |
| 24 | 0.86 | 0.98 | 0.88 | 0.8 | 0.07 | 1 | 0.07 | 0.88 | 0.12 |
| 25 | 0.85 | 0.98 | 0.88 | 0.77 | 0.06 | 1 | 0.06 | 0.88 | 0.11 |
| 26 | 0.86 | 0.98 | 0.81 | 0.85 | 0.08 | 1 | 0.08 | 0.81 | 0.15 |
| 27 | 0.83 | 0.98 | 0.75 | 0.82 | 0.07 | 0.99 | 0.07 | 0.75 | 0.12 |
| 28 | 0.87 | 0.98 | 0.88 | 0.8 | 0.07 | 1 | 0.07 | 0.88 | 0.13 |
| 29 | 0.86 | 0.98 | 0.88 | 0.76 | 0.06 | 1 | 0.06 | 0.88 | 0.11 |
| 30 | 0.86 | 0.98 | 0.81 | 0.83 | 0.07 | 1 | 0.07 | 0.81 | 0.13 |
| 31 | 0.83 | 0.98 | 0.88 | 0.7 | 0.05 | 1 | 0.05 | 0.88 | 0.09 |
| 32 | 0.86 | 0.98 | 0.88 | 0.8 | 0.07 | 1 | 0.07 | 0.88 | 0.12 |
| 33 | 0.86 | 0.98 | 0.88 | 0.8 | 0.07 | 1 | 0.07 | 0.88 | 0.13 |

Table legend: see above.

### Display of AUC and Calibration Plot for the Minimal and Full Models.

The discriminatory ability of models to predict death are displayed using area-under-curve (AUC).

Performance in the Entire Test Set for Individual Prediction of all-Cause Death

All models were constructed and trained in 3,928 eligible individuals and were subsequently tested in all 982 individuals in the test set (2500 randomly selected) to yield the probability for the occurrence of all-cause death at either 365 days or at 180 days. The predicted probabilities following application of all models including the information whether the endpoint was present or absent are listed in Tables 7 and 8.

TABLE 7
Tabulation of individually predicted probabilities for death at 365
days. Note the table is truncated after the first 25 of 982 individuals
and is shown only for the first 5 models including the minimal and
the full model. A complete list on all xxx patients showing the minimal
and the full model output can be found in the supplements.

| ID | Status | Min | Full | model 3 | model 4 | model 5 |
|---|---|---|---|---|---|---|
| 3 | survived | 0.02 | 0.011 | 0.018 | 0.006 | 0.018 |
| 15 | survived | 0.018 | 0.005 | 0.016 | 0.003 | 0.015 |
| 25 | survived | 0.021 | 0.014 | 0.019 | 0.024 | 0.017 |
| 37 | survived | 0.024 | 0.05 | 0.024 | 0.033 | 0.03 |
| 76 | survived | 0.029 | 0.114 | 0.034 | 0.103 | 0.042 |
| 81 | survived | 0.026 | 0.021 | 0.029 | 0.037 | 0.024 |
| 87 | survived | 0.02 | 0.017 | 0.018 | 0.021 | 0.018 |
| 91 | survived | 0.017 | 0.005 | 0.014 | 0.003 | 0.014 |
| 100 | survived | 0.021 | 0.01 | 0.02 | 0.007 | 0.018 |
| 102 | survived | 0.029 | 0.07 | 0.034 | 0.186 | 0.028 |
| 106 | survived | 0.024 | 0.01 | 0.025 | 0.012 | 0.021 |
| 107 | survived | 0.026 | 0.025 | 0.028 | 0.029 | 0.024 |
| 111 | survived | 0.027 | 0.029 | 0.029 | 0.041 | 0.028 |
| 114 | survived | 0.023 | 0.006 | 0.024 | 0.006 | 0.02 |
| 118 | survived | 0.021 | 0.008 | 0.02 | 0.005 | 0.017 |
| 125 | survived | 0.023 | 0.024 | 0.023 | 0.026 | 0.02 |
| 139 | survived | 0.022 | 0.012 | 0.022 | 0.011 | 0.017 |
| 140 | survived | 0.028 | 0.029 | 0.031 | 0.019 | 0.043 |
| 141 | survived | 0.018 | 0.006 | 0.016 | 0.003 | 0.014 |
| 150 | survived | 0.019 | 0.009 | 0.018 | 0.003 | 0.019 |
| 152 | survived | 0.023 | 0.019 | 0.024 | 0.019 | 0.022 |
| 159 | survived | 0.023 | 0.018 | 0.023 | 0.01 | 0.021 |
| 164 | survived | 0.031 | 0.047 | 0.036 | 0.058 | 0.04 |
| 169 | survived | 0.02 | 0.025 | 0.018 | 0.022 | 0.017 |
| 171 | survived | 0.024 | 0.038 | 0.024 | 0.013 | 0.035 |

Table legend: The predicted probabilities for death at 1 year ranged from 1.8% to 3.1% in the minimal model and from 0.5% to 4.7% in the displayed patients of whom no individual actually died.
Note:
the three models that are displayed along with the full and the minimal model comprise the following (from left to right):
minimal_model + ekg_sinus_normal
minimal_model + t0_na_value
minimal_model + t0_hb_value

TABLE 8
Tabulation of individually predicted probabilities for death at
180 days. Note the table is truncated after the first 25 of 982
individuals. A complete list on all 982 patients showing the minimal
and the full model output can be found in the supplements.

| ID | Status | Min | Full | model 3 | model 4 | model 5 |
|---|---|---|---|---|---|---|
| 3 | survived | 0.006 | 0.01 | 0.007 | 0.004 | 0.009 |
| 15 | survived | 0.002 | 0.006 | 0.002 | 0.002 | 0.005 |
| 25 | survived | 0.006 | 0.014 | 0.006 | 0.016 | 0.007 |
| 37 | survived | 0.013 | 0.029 | 0.014 | 0.022 | 0.029 |
| 76 | survived | 0.053 | 0.063 | 0.056 | 0.106 | 0.056 |
| 81 | survived | 0.034 | 0.019 | 0.03 | 0.031 | 0.016 |
| 87 | survived | 0.008 | 0.02 | 0.008 | 0.023 | 0.01 |
| 91 | survived | 0.002 | 0.006 | 0.003 | 0.002 | 0.005 |
| 100 | survived | 0.005 | 0.012 | 0.004 | 0.005 | 0.008 |
| 102 | survived | 0.048 | 0.025 | 0.051 | 0.108 | 0.019 |
| 106 | survived | 0.015 | 0.01 | 0.014 | 0.007 | 0.01 |
| 107 | survived | 0.024 | 0.014 | 0.026 | 0.016 | 0.015 |
| 111 | survived | 0.043 | 0.022 | 0.038 | 0.037 | 0.023 |
| 114 | survived | 0.009 | 0.008 | 0.008 | 0.004 | 0.009 |
| 118 | survived | 0.006 | 0.008 | 0.006 | 0.003 | 0.007 |
| 125 | survived | 0.013 | 0.022 | 0.012 | 0.017 | 0.01 |
| 139 | survived | 0.009 | 0.011 | 0.01 | 0.007 | 0.007 |
| 140 | survived | 0.045 | 0.027 | 0.04 | 0.017 | 0.065 |
| 141 | survived | 0.002 | 0.006 | 0.003 | 0.002 | 0.005 |
| 150 | survived | 0.006 | 0.012 | 0.006 | 0.004 | 0.011 |
| 152 | survived | 0.015 | 0.018 | 0.013 | 0.016 | 0.016 |
| 159 | survived | 0.011 | 0.011 | 0.013 | 0.006 | 0.012 |
| 164 | survived | 0.059 | 0.023 | 0.062 | 0.035 | 0.045 |
| 169 | survived | 0.007 | 0.019 | 0.008 | 0.023 | 0.008 |
| 171 | survived | 0.016 | 0.032 | 0.018 | 0.014 | 0.047 |

Table legend: The predicted probabilities for death at 180 days ranged from 0.2% to 5.9% in the minimal model and from 0.6% to 6.3% in the displayed patients of whom no individual actually died.
Note:
the three models that are displayed along with the full and the minimal model comprise the following (from left to right):
minimal_model + ekg_sinus_normal
minimal_model + t0_na_value
minimal_model + t0_hb_value

### Case Study

Case study: case #505 was classified into the category “rule-out” per ESC 0/1 hour algorithm and a low risk defined by the GRACE score of 84 points for subsequent death was calculated using the GRACE score version 1.0. The patient was transferred to a peripheral hospital. At day 52 the patient died due to a non-cardiac cause. The ML-based minimal and full models had predicted a risk of 1.9% and 3.5% for death at 180 days, respectively indicating a superior prediction of risk for death with the full model, as compared to the established predictive GRACE score.

### Example 3-1: GRACE Score Versus the New Models

Several validated clinical scores that reflect individual risk have been proposed. Among these, the 2020 ESC Guidelines on Acute Coronary Syndromes without ST segment elevation propose the GRACE score as the preferred clinical score and assign a class IIa recommendation (should be considered). The GRACE score integrates patient's age, the occurrence of pre-hospital resuscitation, the presence of pulmonary congestion, impaired renal function into a sum score. The sum score, i.e. less than 109 points, 109 to 139 points, and 140 points or more are interpreted as low, intermediate or risk for the development of death at 180 days to 1 year. This model performance of the GRACE score was tested in the test set regarding its ability to predict death at 180 and 365 days. In the ROC analysis AUC were compared statistically using the method proposed by DeLong.

The AUC of the GRACE score for prediction of death at 180 days was 0.774 (95% CI: 0.65-0.90) (see FIG. 5). The AUC of the minimal model was 0.821 yielding a delta AUC of 0.05. This difference trended to be higher (p=0.085) for the minimal model compared to the GRACE score. The AUC of the full model was 0.86 (95% CI: 0.74-0.98) yielding a large albeit not significant (p=0.11) delta AUC of 0.15.

Likewise, the performance of the GRACE score was compared to the minimal and the full model to predict death at 365 days. Here, the AUC of the full model was 0.86 (95% CI:0.80-0.92) and thus significantly higher (p=0.02) than the AUC of 0.79 (95% CI: 0.72-0.87) of the GRACE score.

### Example 4: Algorithm Obstructive CAD

### Model Selection

Step 1. Correlation and Discrimination within the Derivation Cohort.

The ML algorithm was calibrated by comparing predicted (fitted) probability for the independent test population versus the actual average values regarding the endpoint obstructive CAD requiring revascularization therapies. The calibration found a well-calibrated almost linear relationship between predicted probability for the presence of an obstructed CAD and the actual detection of obstructive CAD requiring revascularization across the entire probability space.

For model selection, following points were taken into consideration:

### 1) Patient Population

### Rules Applied:

From clinical perspective, obstructive CAD is found in patients of all classifications—rule in, observation zone and rule out. All patients receiving coronary angiography within 30 days after presentation were included (N=1,344). Patients without coronary angiography within 30 days after admission were excluded.

### 2) Outcome Variables Timing and Outcome Measure

### Outcome:

The definition of obstructive coronary artery disease requiring revascularization is defined in the preceding sections. Clinically, probability of obstructive CAD is a relevant outcome, since it may influence further diagnostic workup, therapy, as well as their timing.

### 3) Predictors Variables: Demographic, Clinical, Vital Signs, ECG and Laboratory

Different models were tested. The best performing model was well calibrated and showed a moderate diagnostic performance in the test set AUC 0.71 (95% confidence interval 0.708-0.781). Predictor variables were age, sex, creatinine, ECG, and laboratory values (including renal function, hs-cTnT and hs-cTnT kinetics).

TABLE 9
Overview on model equations for prediction of obstructive CAD requiring revascularization
within 30 days showing all 33 models ranging from the minimal model to the full model.

| model | (Intercept) | Gender | Crea | Age: Trop | Age | ΔTρoπ | Smoking | Trop | CP | Revasc |
|---|---|---|---|---|---|---|---|---|---|---|
| min | 0.128 | −0.741 | 0.049 | 0.078 | 0.119 | 0.125 | 0.332 | 0.476 | 0.726 | 0.746 |
| full | 0.272 | −0.614 | 0 | 0 | 0.001 | 0.001 | 0.032 | 0.421 | 0.563 | 0.636 |
| 3 | 0.169 | −0.741 | 0.048 | 0.076 | 0.111 | 0.123 | 0.33 | 0.469 | 0.732 | 0.742 |
| 4 | 0.196 | −0.653 | 0.062 | 0.053 | 0.097 | 0.098 | 0.27 | 0.395 | 0.617 | 0.641 |
| 5 | 0.18 | −0.613 | 0.06 | 0.046 | 0.084 | 0.091 | 0.26 | 0.379 | 0.581 | 0.497 |
| 6 | 0.076 | −0.8 | 0.026 | 0.095 | 0.132 | 0.145 | 0.365 | 0.536 | 0.793 | 0.769 |
| 7 | 0.137 | −0.726 | 0.04 | 0.066 | 0.105 | 0.102 | 0.294 | 0.47 | 0.713 | 0.735 |
| 8 | 0.163 | −0.74 | 0.047 | 0.076 | 0.11 | 0.122 | 0.329 | 0.469 | 0.731 | 0.74 |
| 9 | 0.133 | −0.734 | 0.042 | 0.076 | 0.104 | 0.12 | 0.334 | 0.477 | 0.732 | 0.644 |
| 10 | 0.25 | −0.639 | 0 | 0 | 0.016 | 0.005 | 0.058 | 0.445 | 0.589 | 0.669 |
| 11 | 0.302 | −0.59 | 0 | 0 | 0.008 | 0.002 | 0.022 | 0.398 | 0.521 | 0.618 |
| 12 | 0.194 | −0.579 | 0.061 | 0.039 | 0.076 | 0.084 | 0.238 | 0.351 | 0.541 | 0.461 |
| 13 | 0.079 | −0.799 | 0.019 | 0.091 | 0.125 | 0.128 | 0.346 | 0.541 | 0.791 | 0.779 |
| 14 | 0.133 | −0.726 | 0.04 | 0.066 | 0.104 | 0.102 | 0.293 | 0.469 | 0.712 | 0.734 |
| 15 | 0.156 | −0.644 | 0.052 | 0.051 | 0.09 | 0.097 | 0.277 | 0.402 | 0.609 | 0.479 |
| 16 | 0.108 | −0.719 | 0.035 | 0.066 | 0.098 | 0.1 | 0.298 | 0.477 | 0.71 | 0.64 |
| 17 | 0.275 | −0.583 | 0.042 | 0.014 | 0.061 | 0.053 | 0.161 | 0.351 | 0.52 | 0.521 |
| 18 | 0.333 | −0.557 | 0 | 0 | 0 | 0 | 0 | 0.384 | 0.476 | 0.589 |
| 19 | 0.25 | −0.639 | 0 | 0 | 0.016 | 0.005 | 0.058 | 0.445 | 0.589 | 0.669 |
| 20 | 0.345 | −0.543 | 0.047 | 0.004 | 0.046 | 0.042 | 0.126 | 0.318 | 0.482 | 0.533 |
| 21 | 0.111 | −0.776 | 0.015 | 0.079 | 0.105 | 0.103 | 0.316 | 0.527 | 0.771 | 0.72 |
| 22 | 0.333 | −0.557 | 0 | 0 | 0 | 0 | 0 | 0.384 | 0.476 | 0.589 |
| 23 | 0.25 | −0.639 | 0 | 0 | 0.016 | 0.005 | 0.058 | 0.445 | 0.589 | 0.669 |
| 24 | 0.101 | −0.759 | 0.013 | 0.071 | 0.1 | 0.089 | 0.29 | 0.519 | 0.743 | 0.695 |
| 25 | 0.333 | −0.557 | 0 | 0 | 0 | 0 | 0 | 0.384 | 0.476 | 0.589 |
| 26 | 0.25 | −0.639 | 0 | 0 | 0.016 | 0.005 | 0.058 | 0.445 | 0.589 | 0.669 |
| 27 | 0.209 | −0.666 | 0 | 0.012 | 0.039 | 0.025 | 0.123 | 0.457 | 0.621 | 0.641 |
| 28 | 0.333 | −0.557 | 0 | 0 | 0 | 0 | 0 | 0.384 | 0.476 | 0.589 |
| 29 | 0.333 | −0.557 | 0 | 0 | 0 | 0 | 0 | 0.384 | 0.476 | 0.589 |
| 30 | 0.25 | −0.639 | 0 | 0 | 0.016 | 0.005 | 0.058 | 0.445 | 0.589 | 0.669 |
| 31 | 0.333 | −0.557 | 0 | 0 | 0 | 0 | 0 | 0.384 | 0.476 | 0.589 |
| 32 | 0.206 | −0.674 | 0 | 0.013 | 0.038 | 0.023 | 0.122 | 0.466 | 0.632 | 0.671 |
| 33 | 0.333 | −0.557 | 0 | 0 | 0 | 0 | 0 | 0.384 | 0.476 | 0.589 |

| model | CKD-EPI | ECG | Diabetes | Dyspnea | CAD | MI | Urea | Penalty |
|---|---|---|---|---|---|---|---|---|
| min | | | | | | | | 0.019 |
| full | 0 | 0 | 0 | 0 | 0.006 | 0.02 | 0.031 | 0.019 |
| 3 | | −0.1 | | | | | | 0.019 |
| 4 | | | 0.045 | | | | | 0.049 |
| 5 | | | | | 0.195 | | | 0.061 |
| 6 | | | | | | 0.117 | | 0.004 |
| 7 | | | | 0.017 | | | | 0.019 |
| 8 | | −0.098 | 0.024 | | | | | 0.019 |
| 9 | | −0.094 | | | 0.142 | | | 0.019 |
| 10 | | 0 | | | | 0.03 | | 0.015 |
| 11 | | −0.001 | | 0 | | | | 0.031 |
| 12 | | | 0.046 | | 0.198 | | | 0.077 |
| 13 | | | 0 | | | 0.098 | | 0.002 |
| 14 | | | 0.016 | 0.017 | | | | 0.019 |
| 15 | | | | | 0.168 | 0.182 | | 0.049 |
| 16 | | | | 0.011 | 0.136 | | | 0.019 |
| 17 | | | | 0 | | 0.172 | | 0.061 |
| 18 | | 0 | 0 | | 0 | | | 0.024 |
| 19 | | 0 | 0 | | | 0.03 | | 0.015 |
| 20 | | −0.06 | 0.001 | 0 | | | | 0.077 |
| 21 | | −0.064 | | | 0.06 | 0.083 | | 0.004 |
| 22 | | 0 | | 0 | 0 | | | 0.024 |
| 23 | | 0 | | 0 | | 0.03 | | 0.015 |
| 24 | | | 0 | | 0.069 | 0.091 | | 0.006 |
| 25 | | | 0 | 0 | 0 | | | 0.024 |
| 26 | | | 0 | 0 | | 0.03 | | 0.015 |
| 27 | | | | 0 | 0.047 | 0.059 | | 0.015 |
| 28 | | 0 | 0 | | 0 | 0 | | 0.024 |
| 29 | | 0 | 0 | 0 | 0 | | | 0.024 |
| 30 | | 0 | 0 | 0 | | 0.03 | | 0.015 |
| 31 | | 0 | | 0 | 0 | 0 | | 0.024 |
| 32 | | | 0 | 0 | 0.029 | 0.046 | | 0.012 |
| 33 | | 0 | 0 | 0 | 0 | 0 | | 0.024 |

Table legend: The table shows the individual model equations and the respective parameter with its corresponding relative weight. The minimal model contains 8 different parameters. The full model contains 15 different parameters.
All models as appear from top to bottom are listed regarding their parameters below:
minimal_model
full_model
minimal_model + ekg_sinus_normal
minimal_model + h_diabetes
minimal_model + h_khk
minimal_model + h_infarkt
minimal_model + leading_symptom_dyspnea
minimal_model + ekg_sinus_normal + h_diabetes
minimal_model + ekg_sinus_normal + h_khk
minimal_model + ekg_sinus_normal + h_infarkt
minimal_model + ekg_sinus_normal + leading_symptom_dyspnea
minimal_model + h_diabetes + h_khk
minimal_model + h_diabetes + h_infarkt
minimal_model + h_diabetes + leading_symptom_dyspnea
minimal_model + h_khk + h_infarkt
minimal_model + h_khk + leading_symptom_dyspnea
minimal_model + h_infarkt + leading_symptom_dyspnea
minimal_model + ekg_sinus_normal + h_diabetes + h_khk
minimal_model + ekg_sinus_normal + h_diabetes + h_infarkt
minimal_model + ekg_sinus_normal + h_diabetes + leading_symptom_dyspnea
minimal_model + ekg_sinus_normal + h_khk + h_infarkt
minimal_model + ekg_sinus_normal + h_khk + leading_symptom_dyspnea
minimal_model + ekg_sinus_normal + h_infarkt + leading_symptom_dyspnea
minimal_model + h_diabetes + h_khk + h_infarkt
minimal_model + h_diabetes + h_khk + leading_symptom_dyspnea
minimal_model + h_diabetes + h_infarkt + leading_symptom_dyspnea
minimal_model + h_khk + h_infarkt + leading_symptom_dyspnea
minimal_model + ekg_sinus_normal + h_diabetes + h_khk + h_infarkt
minimal_model + ekg_sinus_normal + h_diabetes + h_khk + leading_symptom_dyspnea
minimal_model + ekg_sinus_normal + h_diabetes + h_infarkt + leading_symptom_dyspnea
minimal_model + ekg_sinus_normal + h_khk + h_infarkt + leading_symptom_dyspnea
minimal_model + h_diabetes + h_khk + h_infarkt + leading_symptom_dyspnea
minimal_model + ekg_sinus_normal + h_diabetes + h_khk + h_infarkt + leading_symptom_dyspne

### Step 2. Performance of the New Models in the Test Set

The risk estimation for the presence of an obstructive CAD requiring revascularization within 30 days with ML was overall moderate to fair with AUC values ranging between 0.69 and 0.71. A summary of the performance of all MVL algorithms including information on AUC, sensitivities, specificities, negative predictive values, positive predictive values, precision, recall and f1.score are listed in Table 10.

TABLE 10
Overview on performance of models on prediction of
CAD requiring revascularization within 30 days.

| model | AUC | accuracy | sensitivity | specificity | ppv | npv | precision | recall | f1.score |
|---|---|---|---|---|---|---|---|---|---|
| min | 0.71 | 0.68 | 0.62 | 0.71 | 0.81 | 0.49 | 0.81 | 0.62 | 0.7 |
| full | 0.69 | 0.7 | 0.59 | 0.7 | 0.8 | 0.47 | 0.8 | 0.59 | 0.68 |
| 3 | 0.7 | 0.68 | 0.66 | 0.68 | 0.8 | 0.5 | 0.8 | 0.66 | 0.72 |
| 4 | 0.7 | 0.68 | 0.62 | 0.71 | 0.81 | 0.49 | 0.81 | 0.62 | 0.7 |
| 5 | 0.7 | 0.68 | 0.62 | 0.7 | 0.8 | 0.49 | 0.8 | 0.62 | 0.7 |
| 6 | 0.71 | 0.68 | 0.64 | 0.7 | 0.81 | 0.5 | 0.81 | 0.64 | 0.71 |
| 7 | 0.7 | 0.68 | 0.65 | 0.68 | 0.8 | 0.5 | 0.8 | 0.65 | 0.71 |
| 8 | 0.7 | 0.68 | 0.66 | 0.68 | 0.8 | 0.51 | 0.8 | 0.66 | 0.73 |
| 9 | 0.7 | 0.69 | 0.67 | 0.66 | 0.79 | 0.5 | 0.79 | 0.67 | 0.73 |
| 10 | 0.69 | 0.69 | 0.56 | 0.75 | 0.81 | 0.46 | 0.81 | 0.56 | 0.66 |
| 11 | 0.69 | 0.7 | 0.58 | 0.74 | 0.81 | 0.47 | 0.81 | 0.58 | 0.68 |
| 12 | 0.7 | 0.69 | 0.64 | 0.68 | 0.8 | 0.49 | 0.8 | 0.64 | 0.71 |
| 13 | 0.71 | 0.69 | 0.64 | 0.7 | 0.81 | 0.5 | 0.81 | 0.64 | 0.72 |
| 14 | 0.7 | 0.68 | 0.65 | 0.68 | 0.8 | 0.5 | 0.8 | 0.65 | 0.72 |
| 15 | 0.7 | 0.68 | 0.62 | 0.71 | 0.81 | 0.49 | 0.81 | 0.62 | 0.7 |
| 16 | 0.7 | 0.69 | 0.64 | 0.69 | 0.8 | 0.5 | 0.8 | 0.64 | 0.71 |
| 17 | 0.69 | 0.69 | 0.62 | 0.72 | 0.81 | 0.49 | 0.81 | 0.62 | 0.7 |
| 18 | 0.69 | 0.69 | 0.65 | 0.66 | 0.79 | 0.49 | 0.79 | 0.65 | 0.71 |
| 19 | 0.69 | 0.69 | 0.56 | 0.75 | 0.81 | 0.46 | 0.81 | 0.56 | 0.66 |
| 20 | 0.69 | 0.7 | 0.61 | 0.7 | 0.8 | 0.48 | 0.8 | 0.61 | 0.7 |
| 21 | 0.7 | 0.69 | 0.66 | 0.68 | 0.8 | 0.51 | 0.8 | 0.66 | 0.72 |
| 22 | 0.69 | 0.69 | 0.65 | 0.66 | 0.79 | 0.49 | 0.79 | 0.65 | 0.71 |
| 23 | 0.69 | 0.69 | 0.56 | 0.75 | 0.81 | 0.46 | 0.81 | 0.56 | 0.66 |
| 24 | 0.7 | 0.69 | 0.65 | 0.69 | 0.81 | 0.5 | 0.81 | 0.65 | 0.72 |
| 25 | 0.69 | 0.69 | 0.65 | 0.66 | 0.79 | 0.49 | 0.79 | 0.65 | 0.71 |
| 26 | 0.69 | 0.69 | 0.56 | 0.75 | 0.8 | 0.46 | 0.81 | 0.56 | 0.66 |
| 27 | 0.7 | 0.68 | 0.55 | 0.76 | 0.82 | 0.47 | 0.82 | 0.55 | 0.66 |
| 28 | 0.69 | 0.69 | 0.65 | 0.66 | 0.79 | 0.49 | 0.79 | 0.65 | 0.71 |
| 29 | 0.69 | 0.69 | 0.65 | 0.66 | 0.79 | 0.49 | 0.79 | 0.65 | 0.71 |
| 30 | 0.69 | 0.69 | 0.56 | 0.75 | 0.81 | 0.46 | 0.81 | 0.56 | 0.66 |
| 31 | 0.69 | 0.69 | 0.65 | 0.66 | 0.79 | 0.49 | 0.79 | 0.65 | 0.71 |
| 32 | 0.7 | 0.69 | 0.55 | 0.76 | 0.82 | 0.47 | 0.82 | 0.55 | 0.66 |
| 33 | 0.69 | 0.69 | 0.65 | 0.66 | 0.79 | 0.49 | 0.79 | 0.65 | 0.71 |

Table legend: This table depicts the respective performance parameters that describe the discriminatory ability to predict the presence of significant coronary artery disease requiring myocardial revascularization. The parameters contain AUC (area under the curve), accuracy, sensitivity, specificity, positive predictive value (PPV), negative predictive value (NPV), precision, recall and F1.Score. Overall AUC ranges from 0.69 to 0.71, with PPVs ranging from 79% to 82% and specificities ranging from 66% to 76%. As the purpose of the model is to predict CAD requiring revascularization the NPVs and sensitivities are not important in this scenario.

### Display of AUC and Calibration Plot for Selected Models.

The minimal and full model had AUC of 0.705 and 0.689, respectively, and were well calibrated (FIG. 6) showing almost linear calibration between predicted and observed values.

The full model comprise the following parameters: patient's sex, age, first troponin (c0), delta troponin, creatinine, estimated glomerular filtration rate, urea (Hst), history of smoking, history of previous revascularization, history of coronary heart disease, history of diabetes. EKG, and presence of chest pain or dyspnea as the leading symptom. The minimal model comprise the following parameter: patient's sex, age, first troponin (c0_Tn), delta troponin, creatinine, history of smoking, history of revascularization, and presence of chest pain.

Performance in the Entire Test Set for Individual Prediction of the Presence of a Significant Coronary Artery Disease Requiring Myocardial Revascularization within 30 Days.

All models trained in 1,007 eligible individuals were tested in 337 individuals in the test set (25% randomly selected) to yield the probability for the presence of significant CAD requiring myocardial revascularization within 30 days. The predicted probabilities following application of all models including the information whether the endpoint was present or absent is listed in Table 11.

TABLE 11
Tabulation of individually predicted probabilities for significant
coronary artery disease requiring myocardial revascularization
within 30 days on the patient level. Note the table is truncated
after the first 25 of 337 individuals. A complete list on
all 337 patients showing the minimal and the full model output
can be found in the supplements.

| ID | status | min | full | mod3 | mod4 | mod5 |
|---|---|---|---|---|---|---|
| 4 | obstructive | 0.811 | 0.794 | 0.818 | 0.791 | 0.791 |
| 13 | non-obstructive | 0.802 | 0.791 | 0.794 | 0.784 | 0.786 |
| 21 | obstructive | 0.769 | 0.759 | 0.778 | 0.753 | 0.756 |
| 31 | non-obstructive | 0.726 | 0.711 | 0.712 | 0.73 | 0.726 |
| 49 | non-obstructive | 0.526 | 0.486 | 0.538 | 0.535 | 0.531 |
| 54 | obstructive | 0.312 | 0.384 | 0.322 | 0.363 | 0.359 |
| 72 | obstructive | 0.437 | 0.456 | 0.445 | 0.475 | 0.463 |
| 77 | obstructive | 0.786 | 0.766 | 0.776 | 0.77 | 0.771 |
| 78 | non-obstructive | 0.767 | 0.703 | 0.773 | 0.763 | 0.766 |
| 95 | obstructive | 0.808 | 0.799 | 0.816 | 0.79 | 0.792 |
| 98 | non-obstructive | 0.655 | 0.656 | 0.663 | 0.657 | 0.664 |
| 124 | obstructive | 0.582 | 0.638 | 0.568 | 0.588 | 0.584 |
| 129 | obstructive | 0.756 | 0.743 | 0.764 | 0.745 | 0.747 |
| 155 | non-obstructive | 0.644 | 0.635 | 0.654 | 0.647 | 0.679 |
| 167 | non-obstructive | 0.602 | 0.675 | 0.613 | 0.61 | 0.608 |
| 175 | non-obstructive | 0.407 | 0.434 | 0.39 | 0.437 | 0.438 |
| 186 | non-obstructive | 0.357 | 0.398 | 0.364 | 0.389 | 0.392 |
| 197 | obstructive | 0.797 | 0.771 | 0.787 | 0.781 | 0.781 |
| 207 | obstructive | 0.476 | 0.503 | 0.483 | 0.485 | 0.484 |
| 273 | obstructive | 0.704 | 0.664 | 0.694 | 0.699 | 0.68 |
| 301 | obstructive | 0.475 | 0.528 | 0.486 | 0.501 | 0.5 |
| 338 | obstructive | 0.748 | 0.735 | 0.735 | 0.721 | 0.711 |
| 350 | non-obstructive | 0.732 | 0.724 | 0.739 | 0.738 | 0.735 |
| 363 | obstructive | 0.796 | 0.775 | 0.803 | 0.778 | 0.778 |
| 370 | obstructive | 0.834 | 0.812 | 0.838 | 0.806 | 0.793 |

Table legend: The table shows predicted probabilities for individuals with and without the presence of CAD requiring revascularization. For better overview only 5 of the 33 models are displayed: from left to right: the minimal model, the full model, a minimal model + EKG, minimal model + Diabetes, and minimal model + history of CAD. The predicted value in the minimal model ranges from 29-96% (data are shown in the complete table).
Case study:
Case study: case #304, a 69 year old man with atypical chest pain but a history of CAD was classified into the category “rule-out” per ESC 0/1 hour algorithm and was attributed a low pre-test probability for relevant coronary artery disease of 25% based on the modified Diamond-Forrest prediction tool. The latter integrates age, sex, typicality of symptoms, presence of cardiovascular risk factors and coronary calcium score where available. The patient was admitted to hospital at the discretion of the attending physician and received coronary angiography showing coronary multivessel disease and a tight coronary lesion requiring percutaneous coronary intervention. The ML-based minimal and full models predicted a risk for CAD requiring revascularization of 55% and 65%, respective, highlighting a superior ability for prediction of CAD requiring revascularization than the established modified Diamond-Forrest prediction tool.

### Example 5: ESC Consortium Prediction Tool Versus the New Predictive Models

Current guidelines in the United States and Canada recommend using the Diamond and Forrester model (Diamond G A, et al. New Engl J Med 1979 June 14, 300 (24): 1350-8) or the Duke clinical score (Pryor D B, et al. Ann Intern Med 1993 January 15, 118 (2): 81-90) to estimate the pre-test probability of CAD in patients presenting with stable chest pain. Both models tend to overestimate the pre-test probability of CAD compared to the ESC consortium calculator (Genders T S, et al. BMJ: British Medical Journal 2012 June 12, 344: e3485). This calculator was developed and validated based on more than 5,500 patients from 18 different hospitals across Europe and the United States. The predictive model integrates age, sex, chest pain, diabetes, hypertension, hyperlipidemia, smoking, coronary calcium score available, and coronary calcium score (enter 0 if not available, and accordingly calculates probabilities by age and symptoms, separately for men and women. This model was tested in the test set regarding its ability to predict coronary obstruction requiring revascularization, and the AUC was compared to all models. The AUC of the ESC consortium algorithm showed a moderate discriminatory performance with an AUC of 0.63 (95% confidence interval: 0.57-0.69). The AUC of the minimal model was 0.71 yielding a delta AUC of 0.08, p=0.004) and the AUC of the full model was 0.69 yielding a delta AUC of 0.06, p=0.04) were significantly higher than the AUC of the ESC Consortium score (see FIG. 8 below). All other models showed an AUC between the AUC of the minimal and the full model.

### Supplemental Tables

SUPPLEMENTAL TABLE 1
Display of demographic, clinical and laboratory
parameters split by survival status at 365 days

| | survived | Died | p | test |
|---|---|---|---|---|
| N | 3828 | 100 | | |
| age (mean (SD)) | 60.30 | 76.34 | <0.001 | |
| | (17.12) | (13.42) | | |
| sex_f1_m0 = 1 (%) | 1684 | 49 | 0.371 | |
| | (44.0) | (49.0) | | |
| ekg_sinus_normal = 1 (%) | 2467 | 32 | <0.001 | |
| | (64.4) | (32.0) | | |
| c0_tnt | 7.00 | 18.00 | <0.001 | nonnorm |
| (median [IQR]) | [4.00, 12.00] | [12.00, 29.25] | | |
| c_first_tnt | 7.00 | 17.50 | <0.001 | nonnorm |
| (median [IQR]) | [4.00, 12.00] | [12.00, 31.00] | | |
| delta_c_first_tnt | −0.12 | 0.03 | 0.437 | |
| (mean (SD)) | (1.95) | (2.33) | | |
| t0_crp_value | 1.00 | 13.90 | <0.001 | nonnorm |
| (median [IQR]) | [1.00, 6.40] | [3.80, 37.83] | | |
| t0_got_value | 24.00 | 25.00 | 0.124 | nonnorm |
| (median [IQR]) | [19.00, 31.00] | [20.00, 34.00] | | |
| t0_gpt_value | 21.00 | 18.00 | 0.004 | nonnorm |
| (median [IQR]) | [15.00, 30.00] | [14.00, 26.00] | | |
| t0_ckdepi_value | 84.58 | 66.82 | <0.001 | |
| (mean (SD)) | (23.10) | (24.90) | | |
| t0_krea_value | 0.85 | 0.97 | 0.002 | nonnorm |
| (median [IQR]) | [0.73, 1.02] | [0.74, 1.27] | | |
| t0_hst_value | 28.00 | 37.50 | <0.001 | nonnorm |
| (median [IQR]) | [22.00, 36.00] | [28.00, 50.50] | | |
| t0_ck_value | 108.00 | 75.00 | <0.001 | nonnorm |
| (median [IQR]) | [76.00, 159.00] | [49.50, 113.00] | | |
| t0_hb_value | 13.75 | 12.28 | <0.001 | |
| (mean (SD)) | (1.64) | (1.94) | | |
| t0_leuko_value | 7.88 | 8.03 | 0.173 | nonnorm |
| (median [IQR]) | [6.47, 9.61] | [6.88, 10.46] | | |
| t0_thrombo_value | 230.00 | 224.00 | 0.262 | nonnorm |
| (median [IQR]) | [193.00, 273.00] | [175.50, 276.50] | | |
| t0_na_value | 139.07 | 136.82 | <0.001 | |
| (mean (SD)) | (3.06) | (5.23) | | |
| t0_gluc_value | 105.00 | 117.00 | <0.001 | nonno |
| (median [IQR]) | [95.00, 125.00] | [100.00, 160.50] | | |

SUPPLEMENTAL TABLE 2
Display of demographic, clinical and laboratory
parameters split by survival status at 180 days

| | survived | Died | p | test |
|---|---|---|---|---|
| N | 3863 | 65 | | |
| age (mean (SD)) | 60.43 | 77.26 | <0.001 | |
| | (17.14) | (13.63) | | |
| sex_f1_m0 = 1 (%) | 1695 | 38 | 0.026 | |
| | (43.9) | (58.5) | | |
| ekg_sinus_normal = 1 (%) | 2476 | 23 | <0.001 | |
| | (64.1) | (35.4) | | |
| c0_tnt | 7.00 | 20.00 | <0.001 | nonnorm |
| (median [IQR]) | [4.00, 12.00] | [12.00, 28.00] | | |
| c_first_tnt | 7.00 | 20.00 | <0.001 | nonnorm |
| (median [IQR]) | [4.00, 12.00] | [12.00, 30.00] | | |
| delta_c_first_tnt | −0.12 | 0.04 | 0.510 | |
| (mean (SD)) | (1.96) | (2.28) | | |
| t0_crp_value | 1.00 | 16.95 | <0.001 | nonnorm |
| (median [IQR]) | [1.00, 6.50] | [4.12, 43.80] | | |
| t0_got_value | 24.00 | 24.00 | 0.313 | nonnorm |
| (median [IQR]) | [19.00, 31.00] | [20.00, 32.25] | | |
| t0_gpt_value | 21.00 | 17.00 | 0.001 | nonnorm |
| (median [IQR]) | [15.00, 30.00] | [13.00, 22.25] | | |
| t0_ckdepi_value | 84.45 | 65.42 | <0.001 | |
| (mean (SD)) | (23.16) | (25.00) | | |
| t0_krea_value | 0.85 | 0.91 | 0.043 | nonnorm |
| (median [IQR]) | [0.73, 1.02] | [0.73, 1.32] | | |
| t0_hst_value | 28.00 | 36.00 | <0.001 | nonnorm |
| (median [IQR]) | [22.00, 36.00] | [27.00, 53.00] | | |
| t0_ck_value | 108.00 | 67.00 | <0.001 | nonnorm |
| (median [IQR]) | [76.00, 159.00] | [47.00, 106.00] | | |
| t0_hb_value | 13.74 | 12.02 | <0.001 | |
| (mean (SD)) | (1.65) | (1.97) | | |
| t0_leuko_value | 7.88 | 7.98 | 0.270 | nonnorm |
| (median [IQR]) | [6.47, 9.63] | [6.95, 11.08] | | |
| t0_thrombo_value | 230.00 | 224.00 | 0.622 | nonnorm |
| (median [IQR]) | [192.00, 273.00] | [185.50, 292.75] | | |
| t0_na_value | 139.06 | 136.05 | <0.001 | |
| (mean (SD)) | (3.07) | (5.69) | | |
| t0_gluc_value | 105.00 | 116.50 | 0.001 | nonnorm |
| (median [IQR]) | [95.00, 125.00] | [100.75, 156.75] | | |

SUPPLEMENTAL TABLE 3
Display of demographic, clinical and laboratory parameters split
by obstructive CAD requiring revascularization within 30 days

| | non-obstructive | obstructive | p | test |
|---|---|---|---|---|
| N | 455 | 889 | | |
| age (mean (SD)) | 69.40 | 70.70 | 0.067 | |
| | (13.36) | (11.70) | | |
| sex_f1_m0 = 1 (%) | 206 | 222 | <0.001 | |
| | (45.3) | (25.0) | | |
| c0_tnt | 17.00 | 28.00 | <0.001 | nonnorm |
| (median [IQR]) | [9.00, 38.00] | [13.00, 86.00] | | |
| delta_c_first_tnt | 19.96 | 50.63 | 0.047 | |
| (mean (SD)) | (129.65) | (315.15) | | |
| h_diabetes = 1 (%) | 126 | 290 | 0.087 | |
| | (29.8) | (34.8) | | |
| h_khk = 1 (%) | 185 | 505 | <0.001 | |
| | (40.7) | (56.9) | | |
| h_infarkt = 1 (%) | 71 | 238 | <0.001 | |
| | (15.7) | (26.8) | | |
| h_hypertonie = 1 (%) | 346 | 718 | 0.125 | |
| | (81.2) | (84.8) | | |
| aktiver_raucher = 1 (%) | 69 | 173 | 0.100 | |
| | (18.7) | (23.2) | | |
| h_cholesterin = 1 (%) | 248 | 537 | 0.116 | |
| | (65.8) | (70.6) | | |
| ekg_sinus_normal = 1 (%) | 211 | 369 | 0.093 | |
| | (46.5) | (41.5) | | |
| t0_ckdepi_value | 77.00 | 75.40 | 0.427 | nonnorm |
| (median [IQR]) | [56.00, 88.50] | [54.32, 89.50] | | |
| t0_krea_value | 0.92 | 0.96 | 0.001 | nonnorm |
| (median [IQR]) | [0.76, 1.12] | [0.81, 1.21] | | |
| t0_hst_value | 32.00 | 34.00 | 0.002 | nonnorm |
| (median [IQR]) | [25.00, 41.25] | [27.00, 45.00] | | |
| symptom_thoraxschmerz = 1 (%) | 274 | 653 | <0.001 | |
| | (60.4) | (73.6) | | |
| leading_symptom_dyspnea = 1 (%) | 91 | 132 | 0.020 | |
| | (20.0) | (14.9) | | |
| h_revasc = 1 (%) | 135 | 443 | <0.001 | |
| | (29.8) | (50.1) | | |

SUPPLEMENTAL TABLE 4
Predicted probabilities for death at 365 days using the
minimal and the full model (of a total of 33 models)

| rapID | o_mortality | minimal_model | full_model |
|---|---|---|---|
| 3 | survived | 0.02 | 0.011 |
| 15 | survived | 0.018 | 0.005 |
| 25 | survived | 0.021 | 0.014 |
| 37 | survived | 0.024 | 0.05 |
| 76 | survived | 0.029 | 0.114 |
| 81 | survived | 0.026 | 0.021 |
| 87 | survived | 0.02 | 0.017 |
| 91 | survived | 0.017 | 0.005 |
| 100 | survived | 0.021 | 0.01 |
| 102 | survived | 0.029 | 0.07 |
| 106 | survived | 0.024 | 0.01 |
| 107 | survived | 0.026 | 0.025 |
| 111 | survived | 0.027 | 0.029 |
| 114 | survived | 0.023 | 0.006 |
| 118 | survived | 0.021 | 0.008 |
| 125 | survived | 0.023 | 0.024 |
| 139 | survived | 0.022 | 0.012 |
| 140 | survived | 0.028 | 0.029 |
| 141 | survived | 0.018 | 0.006 |
| 150 | survived | 0.019 | 0.009 |
| 152 | survived | 0.023 | 0.019 |
| 159 | survived | 0.023 | 0.018 |
| 164 | survived | 0.031 | 0.047 |
| 169 | survived | 0.02 | 0.025 |
| 171 | survived | 0.024 | 0.038 |
| 174 | survived | 0.028 | 0.045 |
| 175 | survived | 0.033 | 0.035 |
| 179 | survived | 0.021 | 0.008 |
| 181 | survived | 0.021 | 0.04 |
| 182 | survived | 0.019 | 0.006 |
| 184 | survived | 0.025 | 0.021 |
| 186 | survived | 0.03 | 0.037 |
| 191 | survived | 0.024 | 0.032 |
| 199 | survived | 0.028 | 0.099 |
| 202 | survived | 0.021 | 0.008 |
| 204 | survived | 0.023 | 0.017 |
| 205 | survived | 0.025 | 0.075 |
| 209 | died | 0.033 | 0.091 |
| 217 | survived | 0.023 | 0.017 |
| 229 | survived | 0.017 | 0.007 |
| 231 | survived | 0.022 | 0.011 |
| 234 | survived | 0.025 | 0.011 |
| 245 | survived | 0.017 | 0.007 |
| 246 | survived | 0.022 | 0.009 |
| 275 | survived | 0.027 | 0.043 |
| 277 | survived | 0.022 | 0.012 |
| 287 | survived | 0.026 | 0.014 |
| 290 | survived | 0.028 | 0.049 |
| 307 | died | 0.032 | 0.114 |
| 309 | survived | 0.032 | 0.079 |
| 310 | survived | 0.03 | 0.064 |
| 320 | survived | 0.029 | 0.02 |
| 344 | survived | 0.025 | 0.046 |
| 348 | survived | 0.029 | 0.013 |
| 350 | survived | 0.029 | 0.084 |
| 356 | survived | 0.025 | 0.013 |
| 358 | survived | 0.019 | 0.005 |
| 366 | survived | 0.024 | 0.045 |
| 373 | survived | 0.017 | 0.004 |
| 377 | survived | 0.021 | 0.008 |
| 381 | survived | 0.031 | 0.085 |
| 383 | died | 0.023 | 0.03 |
| 392 | survived | 0.025 | 0.052 |
| 395 | survived | 0.028 | 0.034 |
| 402 | survived | 0.031 | 0.161 |
| 407 | survived | 0.022 | 0.023 |
| 409 | survived | 0.025 | 0.014 |
| 428 | survived | 0.029 | 0.044 |
| 445 | survived | 0.029 | 0.041 |
| 449 | survived | 0.022 | 0.011 |
| 459 | survived | 0.028 | 0.054 |
| 463 | survived | 0.028 | 0.043 |
| 478 | survived | 0.024 | 0.011 |
| 479 | survived | 0.021 | 0.01 |
| 498 | survived | 0.025 | 0.03 |
| 507 | survived | 0.023 | 0.03 |
| 516 | survived | 0.021 | 0.01 |
| 521 | survived | 0.022 | 0.014 |
| 522 | survived | 0.02 | 0.007 |
| 528 | survived | 0.025 | 0.009 |
| 529 | survived | 0.017 | 0.006 |
| 537 | survived | 0.018 | 0.006 |
| 540 | survived | 0.023 | 0.015 |
| 544 | survived | 0.017 | 0.004 |
| 556 | survived | 0.029 | 0.082 |
| 566 | survived | 0.017 | 0.007 |
| 575 | survived | 0.022 | 0.018 |
| 579 | survived | 0.017 | 0.005 |
| 585 | survived | 0.019 | 0.007 |
| 588 | survived | 0.02 | 0.007 |
| 591 | survived | 0.026 | 0.017 |
| 594 | survived | 0.02 | 0.007 |
| 614 | died | 0.032 | 0.192 |
| 619 | survived | 0.027 | 0.016 |
| 626 | survived | 0.023 | 0.01 |
| 639 | survived | 0.028 | 0.025 |
| 643 | died | 0.033 | 0.313 |
| 658 | survived | 0.02 | 0.007 |
| 664 | survived | 0.017 | 0.013 |
| 669 | survived | 0.02 | 0.01 |
| 671 | survived | 0.021 | 0.013 |
| 674 | survived | 0.02 | 0.009 |
| 686 | survived | 0.021 | 0.019 |
| 689 | survived | 0.023 | 0.014 |
| 692 | survived | 0.024 | 0.03 |
| 693 | survived | 0.017 | 0.014 |
| 694 | survived | 0.017 | 0.007 |
| 704 | survived | 0.029 | 0.018 |
| 708 | survived | 0.017 | 0.005 |
| 716 | survived | 0.031 | 0.036 |
| 717 | survived | 0.022 | 0.018 |
| 718 | survived | 0.024 | 0.013 |
| 721 | survived | 0.018 | 0.005 |
| 724 | survived | 0.018 | 0.008 |
| 752 | survived | 0.018 | 0.004 |
| 753 | survived | 0.026 | 0.022 |
| 754 | survived | 0.022 | 0.009 |
| 773 | survived | 0.024 | 0.029 |
| 775 | survived | 0.019 | 0.014 |
| 777 | survived | 0.019 | 0.01 |
| 778 | survived | 0.02 | 0.008 |
| 789 | survived | 0.026 | 0.024 |
| 790 | survived | 0.023 | 0.027 |
| 825 | survived | 0.028 | 0.064 |
| 830 | survived | 0.021 | 0.008 |
| 839 | survived | 0.02 | 0.005 |
| 841 | survived | 0.022 | 0.015 |
| 843 | survived | 0.021 | 0.01 |
| 857 | survived | 0.017 | 0.011 |
| 862 | survived | 0.022 | 0.015 |
| 871 | survived | 0.021 | 0.023 |
| 874 | survived | 0.028 | 0.028 |
| 883 | survived | 0.023 | 0.012 |
| 890 | survived | 0.024 | 0.013 |
| 897 | survived | 0.021 | 0.012 |
| 898 | survived | 0.035 | 0.223 |
| 899 | survived | 0.026 | 0.054 |
| 901 | survived | 0.019 | 0.016 |
| 904 | died | 0.025 | 0.034 |
| 909 | survived | 0.02 | 0.006 |
| 914 | survived | 0.02 | 0.006 |
| 917 | survived | 0.02 | 0.01 |
| 919 | survived | 0.024 | 0.02 |
| 931 | survived | 0.021 | 0.007 |
| 934 | survived | 0.018 | 0.005 |
| 941 | survived | 0.021 | 0.007 |
| 943 | survived | 0.033 | 0.24 |
| 951 | survived | 0.02 | 0.012 |
| 955 | survived | 0.021 | 0.01 |
| 963 | survived | 0.02 | 0.018 |
| 969 | survived | 0.031 | 0.115 |
| 974 | survived | 0.029 | 0.037 |
| 976 | survived | 0.022 | 0.021 |
| 977 | survived | 0.026 | 0.02 |
| 983 | survived | 0.026 | 0.022 |
| 985 | survived | 0.017 | 0.005 |
| 988 | survived | 0.02 | 0.013 |
| 994 | survived | 0.03 | 0.057 |
| 996 | survived | 0.017 | 0.006 |
| 998 | survived | 0.021 | 0.008 |
| 1001 | survived | 0.023 | 0.009 |
| 1019 | survived | 0.017 | 0.017 |
| 1031 | survived | 0.024 | 0.025 |
| 1034 | survived | 0.02 | 0.008 |
| 1035 | died | 0.02 | 0.006 |
| 1036 | survived | 0.021 | 0.012 |
| 1037 | survived | 0.019 | 0.006 |
| 1050 | survived | 0.021 | 0.006 |
| 1055 | survived | 0.02 | 0.008 |
| 1065 | survived | 0.027 | 0.056 |
| 1069 | survived | 0.024 | 0.015 |
| 1083 | survived | 0.017 | 0.005 |
| 1114 | survived | 0.024 | 0.009 |
| 1116 | survived | 0.018 | 0.006 |
| 1129 | survived | 0.03 | 0.14 |
| 1131 | survived | 0.019 | 0.006 |
| 1135 | survived | 0.017 | 0.006 |
| 1140 | survived | 0.021 | 0.006 |
| 1151 | survived | 0.021 | 0.011 |
| 1152 | survived | 0.024 | 0.015 |
| 1163 | survived | 0.024 | 0.021 |
| 1190 | died | 0.023 | 0.025 |
| 1193 | survived | 0.018 | 0.006 |
| 1204 | survived | 0.023 | 0.021 |
| 1212 | survived | 0.019 | 0.007 |
| 1216 | survived | 0.021 | 0.043 |
| 1226 | survived | 0.023 | 0.025 |
| 1228 | survived | 0.018 | 0.008 |
| 1232 | survived | 0.017 | 0.012 |
| 1250 | survived | 0.022 | 0.014 |
| 1257 | survived | 0.022 | 0.009 |
| 1258 | survived | 0.019 | 0.008 |
| 1260 | survived | 0.023 | 0.018 |
| 1270 | survived | 0.019 | 0.004 |
| 1272 | survived | 0.022 | 0.012 |
| 1275 | survived | 0.029 | 0.079 |
| 1280 | survived | 0.018 | 0.007 |
| 1284 | survived | 0.021 | 0.016 |
| 1287 | survived | 0.022 | 0.009 |
| 1300 | survived | 0.032 | 0.053 |
| 1303 | survived | 0.021 | 0.006 |
| 1314 | survived | 0.02 | 0.008 |
| 1315 | survived | 0.017 | 0.008 |
| 1319 | survived | 0.017 | 0.006 |
| 1324 | survived | 0.017 | 0.009 |
| 1339 | survived | 0.023 | 0.024 |
| 1343 | survived | 0.022 | 0.007 |
| 1346 | survived | 0.023 | 0.012 |
| 1354 | survived | 0.031 | 0.052 |
| 1355 | survived | 0.031 | 0.025 |
| 1368 | survived | 0.021 | 0.01 |
| 1391 | survived | 0.02 | 0.02 |
| 1394 | survived | 0.021 | 0.019 |
| 1399 | survived | 0.019 | 0.007 |
| 1402 | survived | 0.031 | 0.114 |
| 1404 | survived | 0.033 | 0.276 |
| 1412 | survived | 0.019 | 0.01 |
| 1416 | survived | 0.017 | 0.008 |
| 1420 | survived | 0.019 | 0.008 |
| 1446 | survived | 0.024 | 0.023 |
| 1454 | survived | 0.022 | 0.012 |
| 1457 | survived | 0.017 | 0.026 |
| 1479 | survived | 0.03 | 0.095 |
| 1483 | survived | 0.034 | 0.1 |
| 1486 | survived | 0.025 | 0.016 |
| 1502 | survived | 0.022 | 0.007 |
| 1510 | survived | 0.019 | 0.006 |
| 1511 | survived | 0.021 | 0.023 |
| 1516 | survived | 0.017 | 0.008 |
| 1521 | survived | 0.026 | 0.025 |
| 1529 | survived | 0.031 | 0.069 |
| 1552 | survived | 0.022 | 0.008 |
| 1568 | survived | 0.021 | 0.007 |
| 1572 | died | 0.035 | 0.167 |
| 1577 | survived | 0.018 | 0.012 |
| 1578 | died | 0.023 | 0.026 |
| 1586 | survived | 0.018 | 0.005 |
| 1599 | survived | 0.017 | 0.007 |
| 1600 | survived | 0.023 | 0.048 |
| 1611 | survived | 0.022 | 0.01 |
| 1612 | survived | 0.028 | 0.058 |
| 1617 | survived | 0.023 | 0.016 |
| 1628 | survived | 0.024 | 0.02 |
| 1630 | survived | 0.021 | 0.008 |
| 1640 | survived | 0.018 | 0.008 |
| 1644 | survived | 0.021 | 0.007 |
| 1646 | survived | 0.02 | 0.007 |
| 1647 | survived | 0.019 | 0.003 |
| 1649 | survived | 0.027 | 0.047 |
| 1657 | survived | 0.023 | 0.009 |
| 1672 | survived | 0.019 | 0.003 |
| 1680 | survived | 0.028 | 0.027 |
| 1690 | survived | 0.018 | 0.013 |
| 1708 | survived | 0.018 | 0.007 |
| 1753 | survived | 0.02 | 0.006 |
| 1755 | survived | 0.025 | 0.013 |
| 1760 | survived | 0.02 | 0.005 |
| 1772 | survived | 0.02 | 0.006 |
| 1777 | survived | 0.03 | 0.045 |
| 1778 | survived | 0.017 | 0.006 |
| 1780 | survived | 0.02 | 0.008 |
| 1783 | survived | 0.023 | 0.011 |
| 1791 | survived | 0.03 | 0.021 |
| 1796 | survived | 0.02 | 0.007 |
| 1801 | survived | 0.02 | 0.008 |
| 1810 | survived | 0.024 | 0.02 |
| 1815 | survived | 0.019 | 0.009 |
| 1817 | survived | 0.024 | 0.024 |
| 1824 | survived | 0.026 | 0.024 |
| 1833 | survived | 0.021 | 0.005 |
| 1835 | survived | 0.02 | 0.011 |
| 1844 | survived | 0.024 | 0.013 |
| 1852 | survived | 0.021 | 0.011 |
| 1855 | survived | 0.023 | 0.009 |
| 1859 | survived | 0.02 | 0.006 |
| 1867 | survived | 0.02 | 0.005 |
| 1874 | survived | 0.026 | 0.015 |
| 1888 | survived | 0.019 | 0.009 |
| 1898 | survived | 0.021 | 0.007 |
| 1906 | survived | 0.02 | 0.006 |
| 1918 | survived | 0.018 | 0.005 |
| 1926 | survived | 0.02 | 0.012 |
| 1933 | survived | 0.026 | 0.025 |
| 1935 | survived | 0.031 | 0.078 |
| 1944 | survived | 0.02 | 0.008 |
| 1953 | survived | 0.021 | 0.007 |
| 1954 | survived | 0.028 | 0.021 |
| 1958 | survived | 0.021 | 0.008 |
| 1966 | survived | 0.018 | 0.006 |
| 1972 | survived | 0.019 | 0.011 |
| 1975 | survived | 0.021 | 0.01 |
| 2022 | survived | 0.018 | 0.009 |
| 2034 | survived | 0.022 | 0.008 |
| 2035 | survived | 0.021 | 0.008 |
| 2036 | survived | 0.027 | 0.033 |
| 2041 | survived | 0.024 | 0.024 |
| 2044 | died | 0.026 | 0.09 |
| 2049 | survived | 0.019 | 0.005 |
| 2071 | survived | 0.023 | 0.009 |
| 2081 | survived | 0.02 | 0.005 |
| 2083 | survived | 0.02 | 0.005 |
| 2086 | survived | 0.03 | 0.07 |
| 2101 | survived | 0.023 | 0.021 |
| 2107 | survived | 0.03 | 0.037 |
| 2111 | survived | 0.022 | 0.015 |
| 2122 | survived | 0.03 | 0.118 |
| 2129 | survived | 0.027 | 0.016 |
| 2137 | survived | 0.019 | 0.006 |
| 2138 | survived | 0.026 | 0.025 |
| 2144 | survived | 0.02 | 0.009 |
| 2145 | survived | 0.019 | 0.006 |
| 2157 | survived | 0.021 | 0.008 |
| 2169 | survived | 0.024 | 0.022 |
| 2179 | survived | 0.023 | 0.018 |
| 2180 | survived | 0.022 | 0.017 |
| 2200 | survived | 0.022 | 0.008 |
| 2209 | survived | 0.019 | 0.005 |
| 2212 | survived | 0.02 | 0.007 |
| 2216 | survived | 0.02 | 0.004 |
| 2220 | survived | 0.023 | 0.01 |
| 2222 | survived | 0.022 | 0.009 |
| 2226 | survived | 0.025 | 0.017 |
| 2237 | survived | 0.019 | 0.006 |
| 2239 | survived | 0.019 | 0.005 |
| 2243 | survived | 0.022 | 0.01 |
| 2248 | survived | 0.021 | 0.021 |
| 2256 | died | 0.027 | 0.071 |
| 2258 | survived | 0.028 | 0.05 |
| 2267 | survived | 0.027 | 0.058 |
| 2268 | survived | 0.017 | 0.006 |
| 2273 | survived | 0.021 | 0.011 |
| 2275 | survived | 0.021 | 0.016 |
| 2276 | survived | 0.02 | 0.053 |
| 2286 | survived | 0.019 | 0.004 |
| 2287 | survived | 0.024 | 0.011 |
| 2303 | survived | 0.017 | 0.008 |
| 2305 | survived | 0.019 | 0.012 |
| 2320 | survived | 0.019 | 0.007 |
| 2326 | survived | 0.02 | 0.007 |
| 2328 | survived | 0.021 | 0.018 |
| 2345 | survived | 0.019 | 0.005 |
| 2370 | survived | 0.018 | 0.01 |
| 2371 | survived | 0.018 | 0.005 |
| 2373 | survived | 0.019 | 0.01 |
| 2379 | survived | 0.02 | 0.005 |
| 2389 | survived | 0.023 | 0.011 |
| 2391 | survived | 0.02 | 0.007 |
| 2392 | survived | 0.018 | 0.01 |
| 2393 | survived | 0.024 | 0.017 |
| 2407 | survived | 0.02 | 0.011 |
| 2421 | survived | 0.032 | 0.081 |
| 2424 | survived | 0.018 | 0.012 |
| 2431 | survived | 0.02 | 0.005 |
| 2438 | survived | 0.018 | 0.009 |
| 2442 | survived | 0.026 | 0.05 |
| 2444 | survived | 0.022 | 0.015 |
| 2455 | survived | 0.018 | 0.006 |
| 2457 | survived | 0.027 | 0.145 |
| 2459 | survived | 0.032 | 0.04 |
| 2461 | survived | 0.027 | 0.087 |
| 2464 | survived | 0.023 | 0.028 |
| 2472 | survived | 0.022 | 0.024 |
| 2475 | survived | 0.026 | 0.011 |
| 2480 | survived | 0.019 | 0.015 |
| 2492 | survived | 0.021 | 0.013 |
| 2496 | survived | 0.02 | 0.007 |
| 2507 | survived | 0.018 | 0.005 |
| 2508 | survived | 0.031 | 0.058 |
| 2511 | survived | 0.032 | 0.067 |
| 2517 | survived | 0.021 | 0.011 |
| 2524 | survived | 0.019 | 0.005 |
| 2528 | survived | 0.019 | 0.007 |
| 2532 | died | 0.022 | 0.009 |
| 2540 | survived | 0.017 | 0.006 |
| 2541 | survived | 0.02 | 0.011 |
| 2556 | survived | 0.022 | 0.012 |
| 2564 | survived | 0.027 | 0.022 |
| 2568 | survived | 0.02 | 0.01 |
| 2584 | survived | 0.019 | 0.005 |
| 2589 | survived | 0.017 | 0.008 |
| 2592 | survived | 0.02 | 0.007 |
| 2607 | survived | 0.021 | 0.01 |
| 2616 | survived | 0.023 | 0.006 |
| 2622 | survived | 0.022 | 0.008 |
| 2625 | survived | 0.019 | 0.007 |
| 2628 | survived | 0.018 | 0.004 |
| 2651 | survived | 0.023 | 0.025 |
| 2664 | survived | 0.02 | 0.007 |
| 2673 | died | 0.02 | 0.028 |
| 2676 | survived | 0.025 | 0.019 |
| 2697 | survived | 0.021 | 0.006 |
| 2701 | died | 0.026 | 0.042 |
| 2707 | survived | 0.019 | 0.004 |
| 2726 | died | 0.026 | 0.034 |
| 2733 | survived | 0.026 | 0.029 |
| 2738 | survived | 0.021 | 0.009 |
| 2739 | survived | 0.021 | 0.008 |
| 2743 | survived | 0.02 | 0.006 |
| 2747 | survived | 0.024 | 0.018 |
| 2749 | survived | 0.018 | 0.01 |
| 2750 | survived | 0.025 | 0.026 |
| 2752 | survived | 0.026 | 0.015 |
| 2757 | survived | 0.024 | 0.015 |
| 2759 | survived | 0.017 | 0.007 |
| 2781 | survived | 0.02 | 0.005 |
| 2789 | survived | 0.026 | 0.08 |
| 2791 | survived | 0.022 | 0.007 |
| 2797 | survived | 0.028 | 0.02 |
| 2800 | survived | 0.022 | 0.011 |
| 2804 | survived | 0.021 | 0.007 |
| 2807 | survived | 0.018 | 0.005 |
| 2811 | survived | 0.02 | 0.01 |
| 2826 | survived | 0.019 | 0.01 |
| 2833 | survived | 0.018 | 0.004 |
| 2852 | survived | 0.02 | 0.007 |
| 2859 | survived | 0.019 | 0.005 |
| 2873 | survived | 0.017 | 0.007 |
| 2884 | survived | 0.024 | 0.014 |
| 2889 | survived | 0.026 | 0.012 |
| 2897 | survived | 0.017 | 0.005 |
| 2901 | survived | 0.029 | 0.04 |
| 2907 | survived | 0.017 | 0.005 |
| 2909 | survived | 0.023 | 0.017 |
| 2912 | survived | 0.021 | 0.013 |
| 2913 | survived | 0.017 | 0.008 |
| 2920 | survived | 0.02 | 0.009 |
| 2948 | survived | 0.027 | 0.038 |
| 2968 | survived | 0.019 | 0.006 |
| 2971 | survived | 0.017 | 0.005 |
| 2974 | survived | 0.017 | 0.005 |
| 2980 | survived | 0.021 | 0.007 |
| 2987 | survived | 0.017 | 0.01 |
| 2989 | survived | 0.025 | 0.015 |
| 3000 | survived | 0.032 | 0.045 |
| 3022 | survived | 0.019 | 0.004 |
| 3024 | survived | 0.019 | 0.009 |
| 3032 | survived | 0.021 | 0.005 |
| 3033 | survived | 0.022 | 0.015 |
| 3039 | survived | 0.019 | 0.016 |
| 3047 | survived | 0.02 | 0.015 |
| 3049 | survived | 0.023 | 0.007 |
| 3058 | survived | 0.024 | 0.019 |
| 3059 | survived | 0.03 | 0.026 |
| 3061 | survived | 0.023 | 0.015 |
| 3068 | survived | 0.019 | 0.005 |
| 3072 | survived | 0.032 | 0.062 |
| 3074 | survived | 0.024 | 0.015 |
| 3079 | survived | 0.019 | 0.015 |
| 3100 | survived | 0.029 | 0.029 |
| 3105 | survived | 0.02 | 0.01 |
| 3107 | survived | 0.017 | 0.008 |
| 3110 | survived | 0.023 | 0.017 |
| 3111 | survived | 0.017 | 0.01 |
| 3114 | survived | 0.019 | 0.005 |
| 3130 | survived | 0.024 | 0.009 |
| 3143 | survived | 0.024 | 0.017 |
| 3156 | died | 0.034 | 0.145 |
| 3158 | survived | 0.021 | 0.007 |
| 3168 | survived | 0.019 | 0.01 |
| 3188 | survived | 0.019 | 0.009 |
| 3196 | died | 0.03 | 0.065 |
| 3203 | survived | 0.021 | 0.011 |
| 3221 | survived | 0.024 | 0.014 |
| 3225 | survived | 0.017 | 0.004 |
| 3226 | survived | 0.017 | 0.007 |
| 3238 | survived | 0.017 | 0.007 |
| 3241 | survived | 0.019 | 0.007 |
| 3252 | survived | 0.028 | 0.173 |
| 3259 | survived | 0.02 | 0.008 |
| 3265 | survived | 0.017 | 0.006 |
| 3270 | survived | 0.018 | 0.005 |
| 3271 | survived | 0.02 | 0.008 |
| 3281 | survived | 0.022 | 0.007 |
| 3298 | died | 0.025 | 0.023 |
| 3314 | survived | 0.017 | 0.006 |
| 3324 | survived | 0.017 | 0.007 |
| 3330 | survived | 0.019 | 0.005 |
| 3334 | survived | 0.022 | 0.03 |
| 3349 | survived | 0.019 | 0.011 |
| 3353 | survived | 0.018 | 0.004 |
| 3359 | survived | 0.017 | 0.008 |
| 3360 | survived | 0.017 | 0.008 |
| 3363 | survived | 0.021 | 0.012 |
| 3370 | survived | 0.018 | 0.005 |
| 3379 | survived | 0.025 | 0.03 |
| 3383 | survived | 0.017 | 0.004 |
| 3385 | survived | 0.019 | 0.007 |
| 3386 | survived | 0.017 | 0.019 |
| 3391 | survived | 0.03 | 0.041 |
| 3397 | survived | 0.023 | 0.024 |
| 3405 | survived | 0.021 | 0.01 |
| 3410 | survived | 0.017 | 0.006 |
| 3420 | survived | 0.022 | 0.068 |
| 3421 | survived | 0.018 | 0.007 |
| 3432 | survived | 0.021 | 0.011 |
| 10033 | survived | 0.017 | 0.007 |
| 10040 | survived | 0.021 | 0.005 |
| 10042 | survived | 0.026 | 0.028 |
| 10056 | survived | 0.022 | 0.014 |
| 10058 | survived | 0.027 | 0.018 |
| 10061 | survived | 0.019 | 0.006 |
| 10080 | survived | 0.028 | 0.041 |
| 10099 | survived | 0.019 | 0.006 |
| 10106 | died | 0.028 | 0.04 |
| 10110 | survived | 0.019 | 0.006 |
| 10112 | survived | 0.019 | 0.016 |
| 10113 | survived | 0.024 | 0.012 |
| 10120 | survived | 0.026 | 0.027 |
| 10133 | survived | 0.017 | 0.008 |
| 10148 | survived | 0.02 | 0.007 |
| 10155 | survived | 0.023 | 0.02 |
| 10161 | survived | 0.021 | 0.012 |
| 10163 | survived | 0.018 | 0.006 |
| 10169 | survived | 0.023 | 0.017 |
| 10175 | survived | 0.017 | 0.007 |
| 10180 | died | 0.026 | 0.049 |
| 10182 | survived | 0.018 | 0.009 |
| 10188 | survived | 0.019 | 0.007 |
| 10189 | survived | 0.034 | 0.113 |
| 10192 | survived | 0.033 | 0.098 |
| 10208 | survived | 0.022 | 0.017 |
| 10220 | survived | 0.019 | 0.005 |
| 10226 | survived | 0.022 | 0.015 |
| 10237 | survived | 0.02 | 0.006 |
| 10239 | died | 0.024 | 0.051 |
| 10248 | survived | 0.021 | 0.008 |
| 10251 | survived | 0.018 | 0.005 |
| 10259 | survived | 0.032 | 0.362 |
| 10260 | survived | 0.02 | 0.014 |
| 10279 | survived | 0.021 | 0.011 |
| 10308 | survived | 0.026 | 0.013 |
| 10317 | survived | 0.024 | 0.018 |
| 10327 | survived | 0.026 | 0.021 |
| 10332 | survived | 0.023 | 0.028 |
| 10340 | survived | 0.019 | 0.011 |
| 10353 | survived | 0.028 | 0.043 |
| 10360 | survived | 0.02 | 0.008 |
| 10377 | survived | 0.023 | 0.032 |
| 10399 | survived | 0.019 | 0.008 |
| 10401 | survived | 0.02 | 0.011 |
| 10411 | survived | 0.017 | 0.01 |
| 10416 | survived | 0.017 | 0.012 |
| 10418 | survived | 0.024 | 0.019 |
| 10423 | survived | 0.02 | 0.013 |
| 10441 | died | 0.025 | 0.061 |
| 10445 | survived | 0.019 | 0.006 |
| 10456 | survived | 0.019 | 0.009 |
| 10477 | survived | 0.017 | 0.007 |
| 10483 | survived | 0.021 | 0.025 |
| 10494 | survived | 0.021 | 0.012 |
| 10504 | survived | 0.021 | 0.019 |
| 10509 | survived | 0.024 | 0.013 |
| 10558 | survived | 0.019 | 0.006 |
| 10605 | survived | 0.032 | 0.059 |
| 10610 | survived | 0.019 | 0.005 |
| 10614 | survived | 0.017 | 0.007 |
| 10615 | survived | 0.017 | 0.011 |
| 10628 | survived | 0.024 | 0.008 |
| 10655 | died | 0.027 | 0.1 |
| 10670 | survived | 0.028 | 0.035 |
| 10676 | survived | 0.023 | 0.021 |
| 10684 | survived | 0.02 | 0.01 |
| 10697 | survived | 0.021 | 0.008 |
| 10704 | survived | 0.024 | 0.022 |
| 10732 | survived | 0.018 | 0.005 |
| 10748 | survived | 0.02 | 0.005 |
| 10750 | survived | 0.025 | 0.015 |
| 10772 | survived | 0.021 | 0.007 |
| 10775 | survived | 0.025 | 0.079 |
| 10780 | survived | 0.025 | 0.017 |
| 10783 | survived | 0.021 | 0.017 |
| 10785 | survived | 0.025 | 0.021 |
| 10803 | survived | 0.017 | 0.007 |
| 10809 | survived | 0.02 | 0.018 |
| 10826 | survived | 0.021 | 0.013 |
| 10828 | survived | 0.017 | 0.008 |
| 10836 | survived | 0.023 | 0.017 |
| 10839 | survived | 0.019 | 0.006 |
| 10843 | survived | 0.019 | 0.01 |
| 10846 | survived | 0.02 | 0.01 |
| 10856 | survived | 0.018 | 0.015 |
| 10864 | survived | 0.025 | 0.013 |
| 10871 | survived | 0.027 | 0.028 |
| 10879 | survived | 0.019 | 0.007 |
| 10893 | survived | 0.02 | 0.007 |
| 10894 | survived | 0.026 | 0.023 |
| 10896 | survived | 0.034 | 0.109 |
| 10899 | survived | 0.027 | 0.028 |
| 10901 | survived | 0.02 | 0.021 |
| 10907 | survived | 0.018 | 0.004 |
| 10908 | survived | 0.019 | 0.005 |
| 10913 | survived | 0.024 | 0.031 |
| 10918 | survived | 0.017 | 0.007 |
| 10922 | survived | 0.02 | 0.005 |
| 10933 | survived | 0.021 | 0.011 |
| 10939 | survived | 0.025 | 0.057 |
| 10940 | survived | 0.025 | 0.096 |
| 10942 | survived | 0.019 | 0.007 |
| 10945 | survived | 0.023 | 0.009 |
| 10951 | survived | 0.021 | 0.007 |
| 10955 | survived | 0.021 | 0.009 |
| 10959 | survived | 0.026 | 0.02 |
| 10974 | survived | 0.023 | 0.014 |
| 10977 | survived | 0.019 | 0.01 |
| 10983 | survived | 0.017 | 0.01 |
| 11017 | survived | 0.022 | 0.014 |
| 11054 | survived | 0.026 | 0.033 |
| 11057 | survived | 0.022 | 0.015 |
| 11061 | survived | 0.021 | 0.049 |
| 11065 | survived | 0.02 | 0.005 |
| 11070 | survived | 0.024 | 0.037 |
| 11079 | survived | 0.031 | 0.025 |
| 11088 | survived | 0.027 | 0.029 |
| 11102 | survived | 0.017 | 0.005 |
| 11104 | survived | 0.017 | 0.013 |
| 11109 | survived | 0.017 | 0.006 |
| 11111 | survived | 0.019 | 0.011 |
| 11113 | survived | 0.027 | 0.039 |
| 11114 | survived | 0.02 | 0.008 |
| 11119 | survived | 0.022 | 0.01 |
| 11129 | survived | 0.029 | 0.058 |
| 11144 | survived | 0.017 | 0.008 |
| 11151 | survived | 0.02 | 0.007 |
| 11156 | survived | 0.023 | 0.018 |
| 11168 | survived | 0.017 | 0.011 |
| 11176 | survived | 0.025 | 0.043 |
| 11178 | survived | 0.022 | 0.014 |
| 11195 | survived | 0.024 | 0.023 |
| 11201 | survived | 0.017 | 0.006 |
| 11212 | survived | 0.02 | 0.018 |
| 11213 | survived | 0.017 | 0.004 |
| 11215 | survived | 0.023 | 0.012 |
| 11221 | survived | 0.017 | 0.006 |
| 11233 | survived | 0.02 | 0.018 |
| 11234 | survived | 0.017 | 0.011 |
| 11235 | survived | 0.017 | 0.007 |
| 11245 | survived | 0.019 | 0.0 |
| 11255 | survived | 0.024 | 0.025 |
| 11270 | died | 0.019 | 0.018 |
| 11274 | survived | 0.02 | 0.007 |
| 11283 | survived | 0.019 | 0.016 |
| 11306 | died | 0.027 | 0.037 |
| 11308 | survived | 0.018 | 0.005 |
| 11336 | survived | 0.03 | 0.132 |
| 11340 | survived | 0.021 | 0.011 |
| 11341 | survived | 0.029 | 0.038 |
| 11365 | survived | 0.028 | 0.022 |
| 11370 | survived | 0.024 | 0.025 |
| 11371 | survived | 0.026 | 0.025 |
| 11384 | died | 0.02 | 0.014 |
| 11386 | survived | 0.02 | 0.013 |
| 11404 | survived | 0.017 | 0.009 |
| 11413 | survived | 0.028 | 0.042 |
| 11427 | survived | 0.027 | 0.03 |
| 11428 | survived | 0.024 | 0.02 |
| 11456 | survived | 0.023 | 0.017 |
| 11471 | survived | 0.017 | 0.006 |
| 11476 | survived | 0.024 | 0.051 |
| 11477 | survived | 0.027 | 0.035 |
| 11482 | survived | 0.023 | 0.01 |
| 11508 | survived | 0.021 | 0.024 |
| 11528 | survived | 0.019 | 0.021 |
| 11534 | survived | 0.02 | 0.007 |
| 11535 | died | 0.023 | 0.025 |
| 11553 | survived | 0.018 | 0.004 |
| 11559 | survived | 0.017 | 0.009 |
| 11570 | survived | 0.017 | 0.004 |
| 11571 | survived | 0.022 | 0.075 |
| 11613 | survived | 0.022 | 0.014 |
| 11641 | survived | 0.022 | 0.013 |
| 11642 | survived | 0.022 | 0.012 |
| 11651 | survived | 0.017 | 0.01 |
| 11659 | survived | 0.017 | 0.008 |
| 11669 | survived | 0.017 | 0.008 |
| 11681 | survived | 0.021 | 0.017 |
| 11722 | survived | 0.02 | 0.011 |
| 11750 | survived | 0.017 | 0.007 |
| 11790 | survived | 0.021 | 0.012 |
| 11825 | survived | 0.019 | 0.007 |
| 11835 | survived | 0.027 | 0.064 |
| 11844 | survived | 0.017 | 0.008 |
| 11883 | survived | 0.024 | 0.017 |
| 11892 | survived | 0.021 | 0.01 |
| 11896 | survived | 0.023 | 0.017 |
| 11897 | survived | 0.018 | 0.007 |
| 11917 | survived | 0.02 | 0.006 |
| 11926 | survived | 0.02 | 0.011 |
| 11928 | survived | 0.021 | 0.008 |
| 11938 | survived | 0.022 | 0.026 |
| 11947 | survived | 0.024 | 0.01 |
| 11949 | survived | 0.021 | 0.013 |
| 11982 | survived | 0.017 | 0.006 |
| 11993 | survived | 0.021 | 0.012 |
| 12007 | survived | 0.022 | 0.024 |
| 12036 | survived | 0.017 | 0.011 |
| 12047 | survived | 0.017 | 0.008 |
| 12048 | survived | 0.02 | 0.006 |
| 12053 | survived | 0.021 | 0.006 |
| 12076 | survived | 0.017 | 0.006 |
| 12081 | survived | 0.019 | 0.014 |
| 12083 | survived | 0.02 | 0.008 |
| 12091 | survived | 0.017 | 0.006 |
| 12092 | survived | 0.022 | 0.011 |
| 12101 | survived | 0.017 | 0.006 |
| 12143 | survived | 0.025 | 0.028 |
| 12150 | survived | 0.022 | 0.011 |
| 12151 | survived | 0.027 | 0.037 |
| 12153 | survived | 0.021 | 0.009 |
| 12169 | survived | 0.022 | 0.01 |
| 12174 | survived | 0.021 | 0.016 |
| 12176 | survived | 0.023 | 0.015 |
| 12185 | survived | 0.027 | 0.047 |
| 12198 | survived | 0.019 | 0.006 |
| 12207 | survived | 0.021 | 0.011 |
| 12211 | survived | 0.019 | 0.007 |
| 12215 | survived | 0.018 | 0.006 |
| 12220 | survived | 0.026 | 0.036 |
| 12222 | survived | 0.018 | 0.007 |
| 12241 | survived | 0.02 | 0.008 |
| 12244 | survived | 0.022 | 0.014 |
| 12262 | survived | 0.023 | 0.019 |
| 12269 | survived | 0.022 | 0.04 |
| 12279 | survived | 0.017 | 0.008 |
| 12289 | survived | 0.023 | 0.015 |
| 12292 | survived | 0.021 | 0.007 |
| 12296 | survived | 0.021 | 0.006 |
| 12298 | survived | 0.028 | 0.067 |
| 12309 | survived | 0.019 | 0.019 |
| 12316 | survived | 0.018 | 0.006 |
| 12327 | survived | 0.017 | 0.009 |
| 12338 | survived | 0.021 | 0.012 |
| 12339 | survived | 0.017 | 0.008 |
| 12340 | survived | 0.023 | 0.018 |
| 12356 | survived | 0.02 | 0.011 |
| 12358 | survived | 0.018 | 0.006 |
| 12383 | survived | 0.019 | 0.014 |
| 12385 | survived | 0.019 | 0.008 |
| 12396 | survived | 0.028 | 0.047 |
| 12397 | survived | 0.027 | 0.044 |
| 12402 | survived | 0.017 | 0.028 |
| 12411 | survived | 0.02 | 0.015 |
| 12415 | survived | 0.019 | 0.004 |
| 12421 | survived | 0.017 | 0.005 |
| 12423 | survived | 0.02 | 0.037 |
| 12435 | survived | 0.02 | 0.022 |
| 12447 | survived | 0.023 | 0.017 |
| 12449 | survived | 0.02 | 0.01 |
| 12457 | survived | 0.025 | 0.017 |
| 12464 | survived | 0.017 | 0.007 |
| 12466 | survived | 0.026 | 0.029 |
| 12479 | survived | 0.017 | 0.009 |
| 12483 | survived | 0.029 | 0.025 |
| 12504 | survived | 0.022 | 0.012 |
| 12508 | survived | 0.02 | 0.008 |
| 12518 | survived | 0.017 | 0.01 |
| 12528 | survived | 0.031 | 0.056 |
| 12530 | survived | 0.024 | 0.038 |
| 12541 | died | 0.029 | 0.033 |
| 12543 | survived | 0.021 | 0.005 |
| 12547 | survived | 0.029 | 0.031 |
| 12559 | survived | 0.024 | 0.016 |
| 12560 | survived | 0.025 | 0.023 |
| 12561 | survived | 0.02 | 0.006 |
| 12577 | died | 0.023 | 0.019 |
| 12593 | survived | 0.02 | 0.008 |
| 12598 | survived | 0.021 | 0.009 |
| 12601 | survived | 0.026 | 0.02 |
| 12608 | survived | 0.026 | 0.021 |
| 12626 | survived | 0.02 | 0.023 |
| 12627 | survived | 0.019 | 0.007 |
| 12629 | survived | 0.017 | 0.005 |
| 12636 | survived | 0.017 | 0.01 |
| 12645 | survived | 0.02 | 0.006 |
| 12651 | survived | 0.022 | 0.015 |
| 12654 | survived | 0.025 | 0.03 |
| 12689 | survived | 0.017 | 0.007 |
| 12691 | survived | 0.026 | 0.03 |
| 12699 | survived | 0.028 | 0.022 |
| 12713 | survived | 0.024 | 0.026 |
| 12744 | survived | 0.02 | 0.006 |
| 12745 | survived | 0.02 | 0.008 |
| 12758 | survived | 0.018 | 0.011 |
| 12763 | survived | 0.021 | 0.012 |
| 12780 | survived | 0.02 | 0.009 |
| 12798 | survived | 0.023 | 0.009 |
| 12809 | survived | 0.02 | 0.007 |
| 12823 | survived | 0.02 | 0.005 |
| 12836 | survived | 0.023 | 0.011 |
| 12848 | survived | 0.024 | 0.029 |
| 12856 | survived | 0.021 | 0.021 |
| 12862 | survived | 0.023 | 0.007 |
| 12871 | survived | 0.019 | 0.019 |
| 12873 | survived | 0.029 | 0.112 |
| 12885 | survived | 0.023 | 0.013 |
| 12895 | survived | 0.02 | 0.009 |
| 12899 | survived | 0.017 | 0.005 |
| 12907 | survived | 0.017 | 0.006 |
| 12915 | survived | 0.021 | 0.019 |
| 12924 | survived | 0.017 | 0.005 |
| 12949 | survived | 0.02 | 0.008 |
| 12950 | survived | 0.025 | 0.044 |
| 12958 | survived | 0.017 | 0.015 |
| 12959 | survived | 0.019 | 0.008 |
| 12962 | survived | 0.02 | 0.007 |
| 12992 | survived | 0.026 | 0.014 |
| 12997 | survived | 0.024 | 0.024 |
| 13009 | survived | 0.02 | 0.006 |
| 13010 | survived | 0.02 | 0.009 |
| 13026 | survived | 0.03 | 0.04 |
| 13027 | survived | 0.017 | 0.008 |
| 13033 | survived | 0.024 | 0.028 |
| 13039 | survived | 0.017 | 0.006 |
| 13043 | survived | 0.022 | 0.016 |
| 13049 | survived | 0.022 | 0.029 |
| 13058 | survived | 0.029 | 0.052 |
| 13090 | survived | 0.021 | 0.018 |
| 13093 | survived | 0.017 | 0.012 |
| 13117 | survived | 0.02 | 0.005 |
| 13118 | survived | 0.022 | 0.016 |
| 13119 | survived | 0.022 | 0.008 |
| 13124 | survived | 0.022 | 0.018 |
| 13136 | died | 0.035 | 0.171 |
| 13138 | survived | 0.02 | 0.009 |
| 13173 | survived | 0.025 | 0.032 |
| 13190 | survived | 0.025 | 0.048 |
| 13195 | survived | 0.019 | 0.005 |
| 13196 | survived | 0.017 | 0.017 |
| 13200 | survived | 0.021 | 0.01 |
| 13203 | survived | 0.025 | 0.015 |
| 13228 | survived | 0.028 | 0.028 |
| 13234 | survived | 0.03 | 0.047 |
| 13241 | survived | 0.02 | 0.007 |
| 13266 | survived | 0.02 | 0.008 |
| 13277 | survived | 0.026 | 0.033 |
| 13281 | survived | 0.022 | 0.016 |
| 13318 | survived | 0.017 | 0.006 |
| 13330 | survived | 0.024 | 0.028 |
| 13348 | survived | 0.02 | 0.027 |
| 13349 | survived | 0.025 | 0.033 |
| 13357 | survived | 0.017 | 0.007 |
| 13363 | survived | 0.026 | 0.017 |
| 13393 | survived | 0.024 | 0.01 |
| 13399 | survived | 0.017 | 0.007 |
| 13405 | survived | 0.023 | 0.016 |
| 13444 | survived | 0.019 | 0.005 |
| 13457 | survived | 0.028 | 0.039 |
| 13465 | survived | 0.019 | 0.008 |
| 13474 | survived | 0.024 | 0.017 |
| 13489 | survived | 0.021 | 0.007 |
| 13513 | survived | 0.026 | 0.043 |
| 13517 | survived | 0.021 | 0.007 |
| 13532 | survived | 0.019 | 0.007 |
| 13537 | survived | 0.021 | 0.008 |
| 13546 | survived | 0.03 | 0.059 |
| 13547 | survived | 0.021 | 0.012 |
| 13558 | survived | 0.019 | 0.008 |
| 13589 | survived | 0.017 | 0.01 |
| 13592 | survived | 0.017 | 0.005 |
| 13594 | survived | 0.019 | 0.006 |
| 13595 | survived | 0.019 | 0.011 |
| 13613 | survived | 0.025 | 0.015 |
| 13635 | survived | 0.026 | 0.016 |
| 13639 | survived | 0.021 | 0.006 |
| 13642 | survived | 0.022 | 0.008 |
| 13679 | survived | 0.018 | 0.011 |
| 13692 | survived | 0.021 | 0.03 |
| 13694 | survived | 0.027 | 0.054 |
| 13697 | survived | 0.019 | 0.015 |
| 13698 | survived | 0.02 | 0.007 |
| 13706 | survived | 0.019 | 0.006 |
| 13715 | survived | 0.022 | 0.012 |
| 13725 | survived | 0.017 | 0.008 |
| 13728 | survived | 0.02 | 0.013 |
| 13731 | survived | 0.026 | 0.018 |
| 13742 | survived | 0.022 | 0.018 |
| 13747 | survived | 0.029 | 0.053 |
| 13748 | survived | 0.02 | 0.01 |
| 13770 | survived | 0.019 | 0.009 |
| 13782 | survived | 0.018 | 0.007 |
| 13794 | survived | 0.018 | 0.005 |
| 13801 | survived | 0.024 | 0.019 |
| 13809 | survived | 0.022 | 0.006 |
| 13810 | survived | 0.019 | 0.005 |
| 13815 | survived | 0.02 | 0.007 |
| 13832 | survived | 0.025 | 0.047 |
| 13844 | survived | 0.021 | 0.011 |
| 13848 | survived | 0.023 | 0.019 |
| 13852 | survived | 0.02 | 0.007 |
| 13853 | survived | 0.024 | 0.015 |
| 13860 | survived | 0.02 | 0.007 |
| 13862 | survived | 0.027 | 0.046 |
| 13865 | survived | 0.019 | 0.005 |
| 13872 | survived | 0.021 | 0.019 |
| 13885 | survived | 0.028 | 0.032 |
| 13886 | survived | 0.027 | 0.053 |
| 13892 | survived | 0.022 | 0.006 |
| 13902 | survived | 0.021 | 0.038 |
| 13915 | survived | 0.019 | 0.009 |
| 13919 | survived | 0.02 | 0.012 |
| 13926 | survived | 0.019 | 0.012 |
| 13929 | survived | 0.017 | 0.007 |
| 13945 | survived | 0.017 | 0.01 |
| 13954 | survived | 0.021 | 0.007 |
| 13957 | survived | 0.022 | 0.015 |
| 13962 | survived | 0.021 | 0.01 |
| 13970 | survived | 0.022 | 0.009 |
| 13971 | survived | 0.02 | 0.007 |
| 13973 | survived | 0.017 | 0.005 |
| 13976 | survived | 0.018 | 0.011 |
| 13994 | survived | 0.022 | 0.013 |
| 14002 | survived | 0.02 | 0.009 |
| 14014 | survived | 0.023 | 0.009 |
| 14019 | survived | 0.027 | 0.069 |
| 14039 | survived | 0.024 | 0.027 |
| 14053 | survived | 0.023 | 0.019 |
| 14065 | survived | 0.02 | 0.007 |
| 14071 | survived | 0.022 | 0.024 |
| 14072 | survived | 0.022 | 0.021 |
| 14074 | survived | 0.028 | 0.038 |
| 14081 | died | 0.032 | 0.121 |
| 14098 | survived | 0.028 | 0.02 |
| 14102 | survived | 0.019 | 0.022 |
| 14105 | survived | 0.02 | 0.006 |
| 14120 | survived | 0.024 | 0.047 |
| 14132 | survived | 0.025 | 0.03 |
| 14134 | survived | 0.02 | 0.006 |
| 14139 | survived | 0.032 | 0.083 |
| 14155 | survived | 0.036 | 0.212 |
| 14161 | survived | 0.022 | 0.014 |
| 14203 | died | 0.03 | 0.055 |
| 14204 | survived | 0.024 | 0.011 |
| 14205 | survived | 0.019 | 0.006 |
| 14211 | survived | 0.017 | 0.011 |
| 14224 | survived | 0.024 | 0.016 |
| 14235 | survived | 0.018 | 0.011 |
| 14259 | survived | 0.023 | 0.032 |
| 14273 | survived | 0.02 | 0.014 |
| 14274 | survived | 0.019 | 0.006 |
| 14276 | survived | 0.017 | 0.006 |
| 14297 | survived | 0.025 | 0.012 |
| 14300 | survived | 0.025 | 0.016 |
| 14302 | survived | 0.019 | 0.005 |
| 14306 | survived | 0.018 | 0.005 |
| 14314 | survived | 0.02 | 0.004 |
| 14329 | survived | 0.021 | 0.009 |
| 14330 | survived | 0.019 | 0.006 |
| 14334 | survived | 0.02 | 0.009 |
| 14337 | survived | 0.022 | 0.016 |
| 14341 | survived | 0.024 | 0.015 |
| 14344 | survived | 0.021 | 0.025 |
| 14345 | survived | 0.019 | 0.029 |
| 14346 | survived | 0.019 | 0.009 |
| 14361 | survived | 0.022 | 0.006 |
| 14393 | died | 0.036 | 0.16 |
| 14422 | survived | 0.025 | 0.015 |
| 14424 | survived | 0.021 | 0.009 |
| 14428 | survived | 0.025 | 0.015 |
| 14437 | survived | 0.024 | 0.011 |
| 14452 | survived | 0.018 | 0.006 |
| 14456 | survived | 0.026 | 0.026 |
| 14460 | survived | 0.02 | 0.006 |
| 14469 | survived | 0.021 | 0.005 |
| 14480 | survived | 0.019 | 0.005 |
| 14490 | survived | 0.018 | 0.007 |
| 14502 | survived | 0.025 | 0.015 |
| 14550 | survived | 0.025 | 0.04 |
| 14557 | survived | 0.03 | 0.069 |
| 14617 | survived | 0.025 | 0.013 |
| 14618 | survived | 0.021 | 0.01 |
| 14625 | survived | 0.02 | 0.011 |
| 14629 | survived | 0.018 | 0.006 |
| 14637 | survived | 0.021 | 0.044 |
| 14643 | survived | 0.022 | 0.012 |
| 14665 | survived | 0.031 | 0.065 |
| 14667 | survived | 0.019 | 0.007 |
| 14669 | died | 0.03 | 0.075 |
| 14675 | survived | 0.024 | 0.013 |
| 14686 | survived | 0.019 | 0.01 |
| 14703 | survived | 0.019 | 0.007 |
| 14704 | survived | 0.021 | 0.007 |
| 14735 | survived | 0.021 | 0.013 |
| 14758 | survived | 0.021 | 0.011 |

SUPPLEMENTAL TABLE 5
Predicted probabilities for death at 180 days using the
minimal and the full model (of a total of 33 models)

| rapID | o_mortality | minimal_model | full_model |
|---|---|---|---|
| 3 | survived | 0.006 | 0.01 |
| 15 | survived | 0.002 | 0.006 |
| 25 | survived | 0.006 | 0.014 |
| 37 | survived | 0.013 | 0.029 |
| 76 | survived | 0.053 | 0.063 |
| 81 | survived | 0.034 | 0.019 |
| 87 | survived | 0.008 | 0.02 |
| 91 | survived | 0.002 | 0.006 |
| 100 | survived | 0.005 | 0.012 |
| 102 | survived | 0.048 | 0.025 |
| 106 | survived | 0.015 | 0.01 |
| 107 | survived | 0.024 | 0.014 |
| 111 | survived | 0.043 | 0.022 |
| 114 | survived | 0.009 | 0.008 |
| 118 | survived | 0.006 | 0.008 |
| 125 | survived | 0.013 | 0.022 |
| 139 | survived | 0.009 | 0.011 |
| 140 | survived | 0.045 | 0.027 |
| 141 | survived | 0.002 | 0.006 |
| 150 | survived | 0.006 | 0.012 |
| 152 | survived | 0.015 | 0.018 |
| 159 | survived | 0.011 | 0.011 |
| 164 | survived | 0.059 | 0.023 |
| 169 | survived | 0.007 | 0.019 |
| 171 | survived | 0.016 | 0.032 |
| 174 | survived | 0.055 | 0.033 |
| 175 | survived | 0.121 | 0.026 |
| 179 | survived | 0.009 | 0.012 |
| 181 | survived | 0.006 | 0.035 |
| 182 | survived | 0.005 | 0.007 |
| 184 | survived | 0.016 | 0.019 |
| 186 | survived | 0.086 | 0.021 |
| 191 | survived | 0.008 | 0.025 |
| 199 | survived | 0.039 | 0.052 |
| 202 | survived | 0.008 | 0.009 |
| 204 | survived | 0.012 | 0.013 |
| 205 | survived | 0.024 | 0.048 |
| 209 | survived | 0.072 | 0.049 |
| 217 | survived | 0.01 | 0.015 |
| 229 | survived | 0.004 | 0.01 |
| 231 | survived | 0.012 | 0.009 |
| 234 | survived | 0.017 | 0.011 |
| 245 | survived | 0.002 | 0.009 |
| 246 | survived | 0.012 | 0.011 |
| 269 | survived | 0.006 | 0.009 |
| 275 | survived | 0.034 | 0.024 |
| 277 | survived | 0.007 | 0.012 |
| 287 | survived | 0.02 | 0.013 |
| 290 | survived | 0.066 | 0.029 |
| 307 | died | 0.123 | 0.054 |
| 309 | survived | 0.068 | 0.03 |
| 310 | survived | 0.037 | 0.042 |
| 320 | survived | 0.042 | 0.017 |
| 344 | survived | 0.02 | 0.027 |
| 348 | survived | 0.021 | 0.012 |
| 350 | survived | 0.03 | 0.041 |
| 356 | survived | 0.022 | 0.015 |
| 358 | survived | 0.003 | 0.007 |
| 366 | survived | 0.015 | 0.031 |
| 369 | survived | 0.007 | 0.02 |
| 373 | survived | 0.002 | 0.005 |
| 377 | survived | 0.01 | 0.011 |
| 381 | survived | 0.061 | 0.034 |
| 383 | survived | 0.009 | 0.021 |
| 392 | survived | 0.032 | 0.045 |
| 395 | survived | 0.048 | 0.022 |
| 402 | survived | 0.091 | 0.069 |
| 407 | survived | 0.015 | 0.021 |
| 409 | survived | 0.031 | 0.015 |
| 428 | survived | 0.042 | 0.022 |
| 445 | survived | 0.049 | 0.019 |
| 449 | survived | 0.007 | 0.014 |
| 459 | survived | 0.028 | 0.036 |
| 463 | survived | 0.024 | 0.022 |
| 478 | survived | 0.014 | 0.014 |
| 479 | survived | 0.008 | 0.011 |
| 498 | survived | 0.018 | 0.028 |
| 507 | survived | 0.018 | 0.022 |
| 516 | survived | 0.006 | 0.009 |
| 521 | survived | 0.01 | 0.018 |
| 522 | survived | 0.006 | 0.011 |
| 528 | survived | 0.021 | 0.008 |
| 529 | survived | 0.002 | 0.007 |
| 537 | survived | 0.004 | 0.009 |
| 540 | survived | 0.021 | 0.012 |
| 544 | survived | 0.003 | 0.007 |
| 556 | survived | 0.042 | 0.041 |
| 566 | survived | 0.003 | 0.008 |
| 575 | survived | 0.007 | 0.015 |
| 585 | survived | 0.004 | 0.01 |
| 588 | survived | 0.004 | 0.009 |
| 591 | survived | 0.023 | 0.014 |
| 594 | survived | 0.007 | 0.011 |
| 614 | died | 0.126 | 0.082 |
| 619 | survived | 0.03 | 0.015 |
| 626 | survived | 0.014 | 0.014 |
| 627 | survived | 0.014 | 0.016 |
| 639 | survived | 0.044 | 0.015 |
| 643 | died | 0.107 | 0.115 |
| 658 | survived | 0.005 | 0.008 |
| 664 | survived | 0.003 | 0.012 |
| 669 | survived | 0.006 | 0.011 |
| 671 | survived | 0.007 | 0.016 |
| 674 | survived | 0.008 | 0.012 |
| 686 | survived | 0.01 | 0.023 |
| 689 | survived | 0.025 | 0.012 |
| 692 | survived | 0.015 | 0.02 |
| 693 | survived | 0.004 | 0.017 |
| 694 | survived | 0.003 | 0.009 |
| 704 | survived | 0.033 | 0.019 |
| 708 | survived | 0.002 | 0.006 |
| 716 | survived | 0.09 | 0.03 |
| 717 | survived | 0.014 | 0.016 |
| 718 | survived | 0.022 | 0.015 |
| 723 | survived | 0.004 | 0.01 |
| 724 | survived | 0.003 | 0.01 |
| 745 | survived | 0.01 | 0.01 |
| 752 | survived | 0.003 | 0.006 |
| 753 | survived | 0.019 | 0.021 |
| 754 | survived | 0.006 | 0.009 |
| 773 | survived | 0.014 | 0.018 |
| 775 | survived | 0.004 | 0.011 |
| 777 | survived | 0.006 | 0.011 |
| 778 | survived | 0.006 | 0.01 |
| 789 | survived | 0.041 | 0.013 |
| 790 | survived | 0.01 | 0.026 |
| 825 | survived | 0.058 | 0.031 |
| 830 | survived | 0.006 | 0.011 |
| 839 | survived | 0.004 | 0.008 |
| 841 | survived | 0.01 | 0.014 |
| 843 | survived | 0.007 | 0.011 |
| 857 | survived | 0.004 | 0.01 |
| 862 | survived | 0.007 | 0.015 |
| 871 | survived | 0.005 | 0.019 |
| 874 | survived | 0.042 | 0.019 |
| 883 | survived | 0.019 | 0.013 |
| 890 | survived | 0.019 | 0.015 |
| 897 | survived | 0.006 | 0.011 |
| 898 | survived | 0.249 | 0.063 |
| 899 | survived | 0.039 | 0.03 |
| 901 | survived | 0.005 | 0.016 |
| 904 | died | 0.029 | 0.02 |
| 914 | survived | 0.009 | 0.009 |
| 917 | survived | 0.006 | 0.01 |
| 919 | survived | 0.028 | 0.014 |
| 931 | survived | 0.007 | 0.009 |
| 934 | survived | 0.003 | 0.006 |
| 941 | survived | 0.005 | 0.007 |
| 943 | survived | 0.079 | 0.088 |
| 949 | survived | 0.009 | 0.016 |
| 951 | survived | 0.007 | 0.011 |
| 955 | survived | 0.011 | 0.012 |
| 963 | survived | 0.005 | 0.013 |
| 969 | survived | 0.095 | 0.059 |
| 974 | survived | 0.065 | 0.026 |
| 976 | survived | 0.009 | 0.015 |
| 977 | survived | 0.033 | 0.02 |
| 983 | survived | 0.022 | 0.014 |
| 985 | survived | 0.002 | 0.006 |
| 988 | survived | 0.007 | 0.014 |
| 994 | survived | 0.044 | 0.036 |
| 996 | survived | 0.003 | 0.01 |
| 998 | survived | 0.009 | 0.01 |
| 1001 | survived | 0.009 | 0.011 |
| 1019 | survived | 0.004 | 0.012 |
| 1031 | survived | 0.02 | 0.019 |
| 1034 | survived | 0.007 | 0.012 |
| 1035 | died | 0.007 | 0.007 |
| 1036 | survived | 0.01 | 0.011 |
| 1037 | survived | 0.004 | 0.007 |
| 1050 | survived | 0.008 | 0.011 |
| 1055 | survived | 0.005 | 0.01 |
| 1065 | survived | 0.045 | 0.031 |
| 1069 | survived | 0.023 | 0.016 |
| 1083 | survived | 0.002 | 0.007 |
| 1114 | survived | 0.009 | 0.013 |
| 1116 | survived | 0.003 | 0.006 |
| 1129 | survived | 0.057 | 0.055 |
| 1131 | survived | 0.003 | 0.007 |
| 1135 | survived | 0.003 | 0.008 |
| 1140 | survived | 0.005 | 0.011 |
| 1151 | survived | 0.009 | 0.01 |
| 1152 | survived | 0.014 | 0.015 |
| 1163 | survived | 0.011 | 0.015 |
| 1193 | survived | 0.005 | 0.008 |
| 1204 | survived | 0.008 | 0.018 |
| 1212 | survived | 0.003 | 0.007 |
| 1216 | survived | 0.013 | 0.026 |
| 1226 | survived | 0.009 | 0.019 |
| 1228 | survived | 0.002 | 0.01 |
| 1232 | survived | 0.004 | 0.011 |
| 1257 | survived | 0.007 | 0.008 |
| 1258 | survived | 0.006 | 0.011 |
| 1260 | survived | 0.014 | 0.021 |
| 1272 | survived | 0.014 | 0.013 |
| 1275 | survived | 0.045 | 0.035 |
| 1280 | survived | 0.004 | 0.01 |
| 1284 | survived | 0.01 | 0.017 |
| 1287 | survived | 0.008 | 0.009 |
| 1300 | survived | 0.088 | 0.035 |
| 1303 | survived | 0.007 | 0.008 |
| 1314 | survived | 0.008 | 0.011 |
| 1324 | survived | 0.004 | 0.011 |
| 1339 | survived | 0.016 | 0.021 |
| 1343 | survived | 0.008 | 0.009 |
| 1346 | survived | 0.015 | 0.014 |
| 1354 | survived | 0.063 | 0.026 |
| 1355 | survived | 0.062 | 0.011 |
| 1368 | survived | 0.008 | 0.011 |
| 1391 | survived | 0.007 | 0.017 |
| 1394 | survived | 0.009 | 0.022 |
| 1399 | survived | 0.004 | 0.009 |
| 1402 | survived | 0.064 | 0.044 |
| 1404 | survived | 0.089 | 0.118 |
| 1412 | survived | 0.003 | 0.009 |
| 1416 | survived | 0.004 | 0.01 |
| 1420 | survived | 0.006 | 0.011 |
| 1446 | survived | 0.02 | 0.02 |
| 1454 | survived | 0.013 | 0.015 |
| 1457 | survived | 0.002 | 0.018 |
| 1479 | survived | 0.063 | 0.053 |
| 1483 | survived | 0.187 | 0.048 |
| 1486 | survived | 0.03 | 0.017 |
| 1502 | survived | 0.015 | 0.008 |
| 1510 | survived | 0.003 | 0.009 |
| 1511 | survived | 0.006 | 0.02 |
| 1516 | survived | 0.004 | 0.01 |
| 1521 | survived | 0.035 | 0.016 |
| 1529 | survived | 0.069 | 0.032 |
| 1552 | survived | 0.007 | 0.007 |
| 1568 | survived | 0.008 | 0.01 |
| 1572 | died | 0.23 | 0.053 |
| 1577 | survived | 0.004 | 0.01 |
| 1578 | survived | 0.016 | 0.021 |
| 1586 | survived | 0.003 | 0.007 |
| 1599 | survived | 0.004 | 0.01 |
| 1600 | survived | 0.008 | 0.031 |
| 1611 | survived | 0.011 | 0.013 |
| 1612 | survived | 0.036 | 0.03 |
| 1617 | survived | 0.015 | 0.013 |
| 1628 | survived | 0.018 | 0.018 |
| 1630 | survived | 0.007 | 0.01 |
| 1640 | survived | 0.004 | 0.012 |
| 1644 | survived | 0.004 | 0.01 |
| 1646 | survived | 0.004 | 0.01 |
| 1647 | survived | 0.002 | 0.006 |
| 1649 | survived | 0.033 | 0.034 |
| 1657 | survived | 0.018 | 0.01 |
| 1672 | survived | 0.002 | 0.005 |
| 1680 | survived | 0.039 | 0.017 |
| 1690 | survived | 0.004 | 0.013 |
| 1708 | survived | 0.003 | 0.008 |
| 1753 | survived | 0.008 | 0.008 |
| 1755 | survived | 0.029 | 0.013 |
| 1760 | survived | 0.005 | 0.007 |
| 1772 | survived | 0.008 | 0.008 |
| 1777 | survived | 0.045 | 0.025 |
| 1778 | survived | 0.002 | 0.007 |
| 1780 | survived | 0.004 | 0.008 |
| 1783 | survived | 0.011 | 0.011 |
| 1791 | survived | 0.056 | 0.015 |
| 1796 | survived | 0.003 | 0.008 |
| 1810 | survived | 0.021 | 0.015 |
| 1815 | survived | 0.004 | 0.009 |
| 1817 | survived | 0.025 | 0.015 |
| 1818 | survived | 0.004 | 0.011 |
| 1824 | survived | 0.035 | 0.015 |
| 1833 | survived | 0.006 | 0.007 |
| 1835 | survived | 0.005 | 0.017 |
| 1844 | survived | 0.019 | 0.01 |
| 1852 | survived | 0.009 | 0.011 |
| 1859 | survived | 0.005 | 0.008 |
| 1867 | survived | 0.009 | 0.007 |
| 1874 | survived | 0.022 | 0.016 |
| 1875 | survived | 0.002 | 0.007 |
| 1888 | survived | 0.006 | 0.009 |
| 1898 | survived | 0.005 | 0.007 |
| 1900 | survived | 0.012 | 0.012 |
| 1906 | survived | 0.005 | 0.009 |
| 1918 | survived | 0.002 | 0.007 |
| 1926 | survived | 0.007 | 0.011 |
| 1933 | survived | 0.023 | 0.016 |
| 1935 | survived | 0.079 | 0.027 |
| 1944 | survived | 0.008 | 0.011 |
| 1953 | survived | 0.008 | 0.008 |
| 1954 | survived | 0.054 | 0.019 |
| 1958 | survived | 0.009 | 0.011 |
| 1966 | survived | 0.002 | 0.007 |
| 1972 | survived | 0.006 | 0.015 |
| 1975 | survived | 0.005 | 0.011 |
| 1982 | survived | 0.004 | 0.009 |
| 2022 | survived | 0.003 | 0.009 |
| 2035 | survived | 0.006 | 0.011 |
| 2036 | survived | 0.056 | 0.021 |
| 2045 | survived | 0.004 | 0.007 |
| 2068 | survived | 0.012 | 0.008 |
| 2071 | survived | 0.012 | 0.01 |
| 2086 | survived | 0.078 | 0.037 |
| 2101 | survived | 0.009 | 0.019 |
| 2107 | survived | 0.062 | 0.019 |
| 2108 | survived | 0.008 | 0.01 |
| 2115 | survived | 0.007 | 0.01 |
| 2116 | survived | 0.021 | 0.016 |
| 2122 | survived | 0.077 | 0.064 |
| 2138 | survived | 0.029 | 0.017 |
| 2143 | survived | 0.004 | 0.009 |
| 2144 | survived | 0.004 | 0.012 |
| 2145 | survived | 0.005 | 0.01 |
| 2157 | survived | 0.007 | 0.01 |
| 2163 | survived | 0.011 | 0.011 |
| 2169 | survived | 0.023 | 0.015 |
| 2175 | survived | 0.016 | 0.012 |
| 2180 | survived | 0.009 | 0.013 |
| 2183 | survived | 0.003 | 0.006 |
| 2209 | survived | 0.004 | 0.007 |
| 2212 | survived | 0.004 | 0.009 |
| 2216 | survived | 0.004 | 0.008 |
| 2222 | survived | 0.013 | 0.011 |
| 2226 | survived | 0.026 | 0.015 |
| 2227 | survived | 0.024 | 0.028 |
| 2233 | survived | 0.016 | 0.013 |
| 2237 | survived | 0.004 | 0.007 |
| 2239 | survived | 0.005 | 0.009 |
| 2248 | survived | 0.011 | 0.016 |
| 2258 | survived | 0.048 | 0.028 |
| 2264 | survived | 0.004 | 0.01 |
| 2267 | survived | 0.032 | 0.056 |
| 2268 | survived | 0.002 | 0.007 |
| 2273 | survived | 0.008 | 0.011 |
| 2275 | survived | 0.006 | 0.016 |
| 2276 | survived | 0.004 | 0.051 |
| 2286 | survived | 0.003 | 0.006 |
| 2287 | survived | 0.022 | 0.014 |
| 2303 | survived | 0.004 | 0.01 |
| 2320 | survived | 0.004 | 0.008 |
| 2328 | survived | 0.008 | 0.018 |
| 2338 | survived | 0.007 | 0.016 |
| 2345 | survived | 0.003 | 0.006 |
| 2369 | survived | 0.008 | 0.01 |
| 2370 | survived | 0.004 | 0.012 |
| 2371 | survived | 0.002 | 0.006 |
| 2373 | survived | 0.006 | 0.013 |
| 2390 | survived | 0.003 | 0.007 |
| 2392 | survived | 0.004 | 0.01 |
| 2393 | survived | 0.023 | 0.019 |
| 2407 | survived | 0.004 | 0.012 |
| 2418 | survived | 0.182 | 0.032 |
| 2421 | survived | 0.066 | 0.047 |
| 2424 | survived | 0.003 | 0.013 |
| 2426 | survived | 0.032 | 0.025 |
| 2431 | survived | 0.004 | 0.007 |
| 2438 | survived | 0.004 | 0.013 |
| 2442 | survived | 0.031 | 0.029 |
| 2455 | survived | 0.003 | 0.008 |
| 2457 | survived | 0.041 | 0.054 |
| 2461 | survived | 0.051 | 0.046 |
| 2464 | survived | 0.01 | 0.028 |
| 2473 | survived | 0.017 | 0.013 |
| 2475 | survived | 0.021 | 0.012 |
| 2492 | survived | 0.005 | 0.014 |
| 2507 | survived | 0.004 | 0.008 |
| 2517 | survived | 0.006 | 0.01 |
| 2521 | survived | 0.037 | 0.027 |
| 2530 | survived | 0.004 | 0.01 |
| 2536 | survived | 0.022 | 0.015 |
| 2540 | survived | 0.004 | 0.008 |
| 2551 | survived | 0.004 | 0.017 |
| 2552 | died | 0.04 | 0.063 |
| 2557 | survived | 0.008 | 0.02 |
| 2564 | survived | 0.044 | 0.02 |
| 2568 | survived | 0.007 | 0.011 |
| 2573 | survived | 0.02 | 0.014 |
| 2574 | survived | 0.003 | 0.007 |
| 2581 | survived | 0.005 | 0.008 |
| 2584 | survived | 0.003 | 0.008 |
| 2589 | survived | 0.004 | 0.009 |
| 2592 | survived | 0.008 | 0.009 |
| 2607 | survived | 0.009 | 0.014 |
| 2616 | survived | 0.009 | 0.008 |
| 2620 | survived | 0.007 | 0.006 |
| 2621 | survived | 0.003 | 0.007 |
| 2627 | survived | 0.003 | 0.013 |
| 2628 | survived | 0.002 | 0.007 |
| 2635 | survived | 0.004 | 0.023 |
| 2646 | survived | 0.007 | 0.013 |
| 2651 | survived | 0.014 | 0.022 |
| 2658 | survived | 0.03 | 0.018 |
| 2673 | died | 0.004 | 0.027 |
| 2692 | survived | 0.012 | 0.017 |
| 2697 | survived | 0.009 | 0.007 |
| 2707 | survived | 0.003 | 0.006 |
| 2719 | survived | 0.075 | 0.033 |
| 2733 | survived | 0.022 | 0.024 |
| 2738 | survived | 0.011 | 0.011 |
| 2739 | survived | 0.009 | 0.01 |
| 2743 | survived | 0.005 | 0.007 |
| 2744 | survived | 0.021 | 0.033 |
| 2747 | survived | 0.013 | 0.018 |
| 2748 | survived | 0.006 | 0.008 |
| 2749 | survived | 0.003 | 0.01 |
| 2752 | survived | 0.022 | 0.016 |
| 2757 | survived | 0.011 | 0.014 |
| 2761 | survived | 0.003 | 0.011 |
| 2781 | survived | 0.006 | 0.008 |
| 2788 | survived | 0.025 | 0.016 |
| 2791 | survived | 0.006 | 0.008 |
| 2792 | survived | 0.011 | 0.01 |
| 2793 | survived | 0.009 | 0.013 |
| 2800 | survived | 0.011 | 0.014 |
| 2804 | survived | 0.007 | 0.008 |
| 2811 | survived | 0.003 | 0.015 |
| 2821 | survived | 0.022 | 0.019 |
| 2824 | survived | 0.02 | 0.014 |
| 2856 | survived | 0.007 | 0.012 |
| 2873 | survived | 0.004 | 0.008 |
| 2884 | survived | 0.012 | 0.012 |
| 2894 | survived | 0.007 | 0.01 |
| 2909 | survived | 0.013 | 0.016 |
| 2912 | survived | 0.008 | 0.014 |
| 2913 | survived | 0.003 | 0.007 |
| 2916 | survived | 0.009 | 0.008 |
| 2920 | survived | 0.004 | 0.012 |
| 2933 | survived | 0.025 | 0.019 |
| 2948 | survived | 0.046 | 0.027 |
| 2983 | survived | 0.007 | 0.028 |
| 2989 | survived | 0.03 | 0.012 |
| 2994 | survived | 0.041 | 0.027 |
| 3011 | survived | 0.007 | 0.018 |
| 3018 | survived | 0.004 | 0.007 |
| 3022 | survived | 0.005 | 0.007 |
| 3024 | survived | 0.003 | 0.011 |
| 3032 | survived | 0.005 | 0.007 |
| 3040 | survived | 0.004 | 0.008 |
| 3047 | survived | 0.006 | 0.014 |
| 3049 | survived | 0.009 | 0.008 |
| 3061 | survived | 0.019 | 0.016 |
| 3068 | survived | 0.003 | 0.007 |
| 3072 | survived | 0.054 | 0.032 |
| 3073 | survived | 0.098 | 0.022 |
| 3074 | survived | 0.025 | 0.011 |
| 3083 | survived | 0.064 | 0.028 |
| 3100 | survived | 0.045 | 0.018 |
| 3107 | survived | 0.002 | 0.009 |
| 3111 | survived | 0.003 | 0.01 |
| 3126 | survived | 0.003 | 0.007 |
| 3142 | survived | 0.03 | 0.013 |
| 3144 | survived | 0.007 | 0.015 |
| 3145 | survived | 0.005 | 0.009 |
| 3151 | survived | 0.006 | 0.011 |
| 3156 | died | 0.099 | 0.054 |
| 3167 | survived | 0.004 | 0.007 |
| 3168 | survived | 0.004 | 0.016 |
| 3183 | survived | 0.01 | 0.008 |
| 3185 | survived | 0.01 | 0.014 |
| 3192 | survived | 0.002 | 0.008 |
| 3196 | died | 0.068 | 0.03 |
| 3216 | survived | 0.007 | 0.007 |
| 3221 | survived | 0.013 | 0.016 |
| 3224 | survived | 0.009 | 0.01 |
| 3225 | survived | 0.003 | 0.007 |
| 3226 | survived | 0.004 | 0.009 |
| 3237 | survived | 0.027 | 0.015 |
| 3241 | survived | 0.004 | 0.009 |
| 3262 | survived | 0.005 | 0.009 |
| 3282 | survived | 0.002 | 0.006 |
| 3298 | died | 0.024 | 0.02 |
| 3304 | survived | 0.005 | 0.01 |
| 3320 | survived | 0.005 | 0.007 |
| 3323 | survived | 0.004 | 0.007 |
| 3330 | survived | 0.005 | 0.009 |
| 3334 | survived | 0.009 | 0.02 |
| 3336 | survived | 0.002 | 0.006 |
| 3343 | survived | 0.02 | 0.012 |
| 3349 | survived | 0.004 | 0.012 |
| 3359 | survived | 0.004 | 0.01 |
| 3360 | survived | 0.003 | 0.008 |
| 3370 | survived | 0.002 | 0.006 |
| 3377 | survived | 0.004 | 0.008 |
| 3383 | survived | 0.003 | 0.007 |
| 3386 | survived | 0.003 | 0.012 |
| 3393 | survived | 0.082 | 0.024 |
| 3397 | survived | 0.021 | 0.019 |
| 3406 | survived | 0.023 | 0.022 |
| 3417 | survived | 0.006 | 0.009 |
| 3421 | survived | 0.003 | 0.007 |
| 3430 | survived | 0.004 | 0.008 |
| 3431 | survived | 0.009 | 0.012 |
| 3433 | survived | 0.002 | 0.006 |
| 10022 | survived | 0.022 | 0.022 |
| 10033 | survived | 0.004 | 0.009 |
| 10036 | survived | 0.027 | 0.016 |
| 10038 | survived | 0.007 | 0.013 |
| 10039 | survived | 0.003 | 0.009 |
| 10040 | survived | 0.007 | 0.009 |
| 10042 | survived | 0.023 | 0.019 |
| 10045 | survived | 0.008 | 0.017 |
| 10056 | survived | 0.009 | 0.013 |
| 10061 | survived | 0.003 | 0.009 |
| 10062 | survived | 0.003 | 0.009 |
| 10079 | survived | 0.007 | 0.011 |
| 10097 | survived | 0.026 | 0.027 |
| 10099 | survived | 0.004 | 0.007 |
| 10106 | died | 0.036 | 0.023 |
| 10113 | survived | 0.02 | 0.012 |
| 10134 | survived | 0.004 | 0.013 |
| 10139 | survived | 0.013 | 0.011 |
| 10144 | survived | 0.014 | 0.028 |
| 10148 | survived | 0.007 | 0.008 |
| 10155 | survived | 0.018 | 0.02 |
| 10161 | survived | 0.009 | 0.012 |
| 10166 | survived | 0.015 | 0.029 |
| 10169 | survived | 0.017 | 0.013 |
| 10175 | survived | 0.003 | 0.01 |
| 10180 | survived | 0.031 | 0.031 |
| 10181 | survived | 0.004 | 0.013 |
| 10186 | survived | 0.009 | 0.012 |
| 10189 | survived | 0.111 | 0.035 |
| 10192 | survived | 0.1 | 0.039 |
| 10208 | survived | 0.007 | 0.017 |
| 10220 | survived | 0.003 | 0.007 |
| 10226 | survived | 0.008 | 0.016 |
| 10246 | survived | 0.002 | 0.006 |
| 10248 | survived | 0.005 | 0.008 |
| 10259 | survived | 0.162 | 0.112 |
| 10260 | survived | 0.004 | 0.017 |
| 10264 | survived | 0.02 | 0.015 |
| 10279 | survived | 0.006 | 0.01 |
| 10292 | survived | 0.046 | 0.02 |
| 10296 | survived | 0.011 | 0.013 |
| 10308 | survived | 0.034 | 0.015 |
| 10310 | survived | 0.021 | 0.02 |
| 10311 | survived | 0.033 | 0.017 |
| 10313 | survived | 0.01 | 0.01 |
| 10317 | survived | 0.017 | 0.017 |
| 10319 | survived | 0.005 | 0.008 |
| 10337 | survived | 0.005 | 0.008 |
| 10340 | survived | 0.005 | 0.017 |
| 10354 | survived | 0.004 | 0.009 |
| 10355 | survived | 0.002 | 0.009 |
| 10376 | survived | 0.007 | 0.011 |
| 10378 | survived | 0.011 | 0.014 |
| 10382 | survived | 0.009 | 0.015 |
| 10387 | survived | 0.023 | 0.015 |
| 10401 | survived | 0.008 | 0.011 |
| 10411 | survived | 0.004 | 0.012 |
| 10416 | survived | 0.003 | 0.012 |
| 10418 | survived | 0.024 | 0.013 |
| 10426 | survived | 0.017 | 0.012 |
| 10445 | survived | 0.006 | 0.01 |
| 10456 | survived | 0.006 | 0.013 |
| 10461 | survived | 0.023 | 0.021 |
| 10466 | survived | 0.006 | 0.011 |
| 10473 | survived | 0.002 | 0.01 |
| 10477 | survived | 0.004 | 0.01 |
| 10504 | survived | 0.008 | 0.013 |
| 10520 | survived | 0.025 | 0.016 |
| 10527 | survived | 0.015 | 0.022 |
| 10558 | survived | 0.003 | 0.009 |
| 10599 | survived | 0.005 | 0.019 |
| 10603 | survived | 0.014 | 0.015 |
| 10605 | survived | 0.068 | 0.046 |
| 10607 | survived | 0.005 | 0.007 |
| 10614 | survived | 0.003 | 0.008 |
| 10628 | survived | 0.017 | 0.008 |
| 10665 | survived | 0.02 | 0.015 |
| 10670 | survived | 0.024 | 0.029 |
| 10672 | survived | 0.006 | 0.011 |
| 10684 | survived | 0.007 | 0.012 |
| 10702 | survived | 0.005 | 0.01 |
| 10704 | survived | 0.011 | 0.017 |
| 10708 | survived | 0.004 | 0.007 |
| 10727 | survived | 0.006 | 0.01 |
| 10748 | survived | 0.007 | 0.007 |
| 10750 | survived | 0.018 | 0.015 |
| 10756 | survived | 0.003 | 0.007 |
| 10768 | survived | 0.004 | 0.013 |
| 10772 | survived | 0.008 | 0.011 |
| 10775 | survived | 0.015 | 0.039 |
| 10780 | survived | 0.024 | 0.018 |
| 10781 | survived | 0.011 | 0.018 |
| 10783 | survived | 0.008 | 0.014 |
| 10785 | survived | 0.017 | 0.014 |
| 10800 | survived | 0.008 | 0.009 |
| 10811 | survived | 0.007 | 0.009 |
| 10826 | survived | 0.008 | 0.013 |
| 10848 | survived | 0.005 | 0.011 |
| 10856 | survived | 0.007 | 0.012 |
| 10864 | survived | 0.018 | 0.013 |
| 10866 | survived | 0.017 | 0.012 |
| 10868 | survived | 0.014 | 0.01 |
| 10879 | survived | 0.004 | 0.01 |
| 10898 | survived | 0.004 | 0.011 |
| 10899 | survived | 0.03 | 0.021 |
| 10902 | survived | 0.002 | 0.007 |
| 10906 | survived | 0.005 | 0.013 |
| 10907 | survived | 0.002 | 0.006 |
| 10909 | survived | 0.011 | 0.008 |
| 10918 | survived | 0.004 | 0.009 |
| 10925 | survived | 0.003 | 0.011 |
| 10939 | survived | 0.021 | 0.029 |
| 10940 | survived | 0.02 | 0.046 |
| 10942 | survived | 0.004 | 0.009 |
| 10945 | survived | 0.008 | 0.012 |
| 10947 | survived | 0.012 | 0.021 |
| 10972 | survived | 0.008 | 0.017 |
| 10977 | survived | 0.005 | 0.009 |
| 10979 | survived | 0.004 | 0.009 |
| 10984 | survived | 0.005 | 0.014 |
| 11006 | survived | 0.004 | 0.012 |
| 11016 | survived | 0.007 | 0.009 |
| 11017 | survived | 0.013 | 0.015 |
| 11018 | survived | 0.003 | 0.01 |
| 11054 | survived | 0.039 | 0.03 |
| 11057 | survived | 0.009 | 0.012 |
| 11070 | survived | 0.012 | 0.025 |
| 11071 | survived | 0.011 | 0.008 |
| 11076 | survived | 0.004 | 0.011 |
| 11094 | survived | 0.002 | 0.007 |
| 11099 | survived | 0.002 | 0.008 |
| 11102 | survived | 0.002 | 0.007 |
| 11109 | survived | 0.003 | 0.007 |
| 11112 | survived | 0.052 | 0.04 |
| 11118 | survived | 0.066 | 0.048 |
| 11123 | survived | 0.017 | 0.024 |
| 11142 | survived | 0.002 | 0.006 |
| 11148 | survived | 0.005 | 0.01 |
| 11163 | survived | 0.013 | 0.012 |
| 11168 | survived | 0.003 | 0.008 |
| 11176 | survived | 0.031 | 0.026 |
| 11178 | survived | 0.008 | 0.016 |
| 11184 | survived | 0.03 | 0.015 |
| 11187 | survived | 0.003 | 0.01 |
| 11189 | survived | 0.007 | 0.018 |
| 11193 | survived | 0.005 | 0.012 |
| 11194 | survived | 0.011 | 0.02 |
| 11213 | survived | 0.002 | 0.007 |
| 11221 | survived | 0.003 | 0.006 |
| 11234 | survived | 0.004 | 0.01 |
| 11235 | survived | 0.004 | 0.009 |
| 11242 | survived | 0.012 | 0.023 |
| 11253 | survived | 0.009 | 0.01 |
| 11255 | survived | 0.014 | 0.018 |
| 11256 | survived | 0.007 | 0.011 |
| 11274 | survived | 0.004 | 0.009 |
| 11283 | survived | 0.005 | 0.017 |
| 11284 | survived | 0.069 | 0.028 |
| 11297 | survived | 0.002 | 0.012 |
| 11308 | survived | 0.002 | 0.007 |
| 11337 | survived | 0.003 | 0.008 |
| 11340 | survived | 0.005 | 0.011 |
| 11341 | survived | 0.042 | 0.033 |
| 11365 | survived | 0.044 | 0.015 |
| 11384 | died | 0.008 | 0.014 |
| 11386 | survived | 0.005 | 0.013 |
| 11391 | survived | 0.004 | 0.007 |
| 11396 | survived | 0.003 | 0.007 |
| 11402 | survived | 0.003 | 0.01 |
| 11416 | survived | 0.006 | 0.014 |
| 11417 | survived | 0.01 | 0.011 |
| 11427 | survived | 0.027 | 0.024 |
| 11429 | survived | 0.024 | 0.022 |
| 11448 | survived | 0.006 | 0.011 |
| 11456 | survived | 0.01 | 0.014 |
| 11486 | survived | 0.008 | 0.011 |
| 11498 | survived | 0.034 | 0.011 |
| 11530 | survived | 0.004 | 0.008 |
| 11557 | survived | 0.015 | 0.012 |
| 11558 | survived | 0.002 | 0.013 |
| 11573 | survived | 0.007 | 0.012 |
| 11576 | survived | 0.002 | 0.015 |
| 11585 | survived | 0.004 | 0.008 |
| 11601 | survived | 0.005 | 0.008 |
| 11610 | survived | 0.01 | 0.018 |
| 11613 | survived | 0.011 | 0.012 |
| 11620 | died | 0.009 | 0.03 |
| 11628 | survived | 0.015 | 0.012 |
| 11640 | survived | 0.02 | 0.024 |
| 11650 | survived | 0.021 | 0.021 |
| 11651 | survived | 0.004 | 0.009 |
| 11661 | survived | 0.013 | 0.015 |
| 11670 | survived | 0.004 | 0.007 |
| 11684 | survived | 0.026 | 0.018 |
| 11691 | survived | 0.062 | 0.033 |
| 11711 | survived | 0.015 | 0.018 |
| 11729 | survived | 0.044 | 0.016 |
| 11737 | survived | 0.07 | 0.031 |
| 11738 | survived | 0.003 | 0.011 |
| 11746 | survived | 0.004 | 0.01 |
| 11750 | survived | 0.002 | 0.009 |
| 11777 | survived | 0.021 | 0.015 |
| 11825 | survived | 0.004 | 0.01 |
| 11833 | survived | 0.015 | 0.013 |
| 11835 | survived | 0.043 | 0.035 |
| 11840 | survived | 0.005 | 0.01 |
| 11868 | survived | 0.004 | 0.016 |
| 11883 | survived | 0.016 | 0.016 |
| 11885 | survived | 0.027 | 0.02 |
| 11890 | survived | 0.03 | 0.012 |
| 11896 | survived | 0.011 | 0.015 |
| 11897 | survived | 0.005 | 0.011 |
| 11952 | survived | 0.012 | 0.025 |
| 11963 | survived | 0.005 | 0.008 |
| 11967 | survived | 0.002 | 0.025 |
| 11980 | survived | 0.005 | 0.015 |
| 11993 | survived | 0.006 | 0.011 |
| 11997 | survived | 0.009 | 0.009 |
| 12006 | survived | 0.007 | 0.017 |
| 12007 | survived | 0.009 | 0.016 |
| 12030 | survived | 0.02 | 0.03 |
| 12034 | survived | 0.006 | 0.01 |
| 12078 | survived | 0.021 | 0.021 |
| 12081 | survived | 0.005 | 0.014 |
| 12088 | survived | 0.073 | 0.029 |
| 12100 | survived | 0.012 | 0.039 |
| 12118 | survived | 0.003 | 0.01 |
| 12150 | survived | 0.007 | 0.009 |
| 12154 | survived | 0.004 | 0.007 |
| 12166 | survived | 0.004 | 0.011 |
| 12169 | survived | 0.012 | 0.012 |
| 12174 | survived | 0.011 | 0.015 |
| 12183 | survived | 0.01 | 0.014 |
| 12185 | survived | 0.023 | 0.029 |
| 12207 | survived | 0.008 | 0.012 |
| 12208 | survived | 0.003 | 0.009 |
| 12211 | survived | 0.004 | 0.008 |
| 12215 | survived | 0.003 | 0.008 |
| 12218 | survived | 0.007 | 0.008 |
| 12220 | survived | 0.028 | 0.024 |
| 12222 | survived | 0.003 | 0.008 |
| 12241 | survived | 0.005 | 0.007 |
| 12244 | survived | 0.008 | 0.015 |
| 12262 | survived | 0.015 | 0.016 |
| 12279 | survived | 0.004 | 0.01 |
| 12281 | survived | 0.004 | 0.01 |
| 12289 | survived | 0.017 | 0.016 |
| 12298 | survived | 0.048 | 0.042 |
| 12299 | survived | 0.01 | 0.016 |
| 12320 | survived | 0.004 | 0.011 |
| 12321 | survived | 0.021 | 0.018 |
| 12332 | survived | 0.029 | 0.026 |
| 12335 | survived | 0.003 | 0.008 |
| 12339 | survived | 0.002 | 0.009 |
| 12340 | survived | 0.013 | 0.012 |
| 12353 | survived | 0.005 | 0.012 |
| 12368 | survived | 0.02 | 0.02 |
| 12371 | survived | 0.059 | 0.032 |
| 12375 | survived | 0.018 | 0.012 |
| 12399 | survived | 0.055 | 0.021 |
| 12402 | survived | 0.005 | 0.023 |
| 12411 | survived | 0.008 | 0.013 |
| 12414 | survived | 0.016 | 0.016 |
| 12416 | survived | 0.004 | 0.017 |
| 12421 | survived | 0.003 | 0.007 |
| 12426 | survived | 0.007 | 0.008 |
| 12443 | survived | 0.009 | 0.009 |
| 12449 | survived | 0.006 | 0.011 |
| 12453 | survived | 0.004 | 0.014 |
| 12457 | survived | 0.022 | 0.01 |
| 12466 | survived | 0.033 | 0.026 |
| 12473 | survived | 0.021 | 0.012 |
| 12479 | survived | 0.004 | 0.012 |
| 12483 | survived | 0.049 | 0.015 |
| 12492 | survived | 0.01 | 0.011 |
| 12504 | survived | 0.012 | 0.014 |
| 12508 | survived | 0.005 | 0.01 |
| 12528 | survived | 0.078 | 0.022 |
| 12549 | survived | 0.004 | 0.009 |
| 12553 | survived | 0.004 | 0.015 |
| 12556 | survived | 0.015 | 0.011 |
| 12563 | survived | 0.007 | 0.013 |
| 12571 | survived | 0.103 | 0.056 |
| 12584 | survived | 0.006 | 0.009 |
| 12601 | survived | 0.03 | 0.013 |
| 12603 | survived | 0.009 | 0.012 |
| 12614 | survived | 0.004 | 0.02 |
| 12628 | survived | 0.002 | 0.012 |
| 12629 | survived | 0.003 | 0.008 |
| 12630 | survived | 0.035 | 0.049 |
| 12656 | survived | 0.008 | 0.015 |
| 12698 | survived | 0.006 | 0.013 |
| 12699 | survived | 0.031 | 0.014 |
| 12702 | survived | 0.014 | 0.017 |
| 12709 | survived | 0.006 | 0.008 |
| 12734 | survived | 0.004 | 0.008 |
| 12744 | survived | 0.005 | 0.007 |
| 12751 | survived | 0.067 | 0.018 |
| 12763 | survived | 0.007 | 0.01 |
| 12771 | survived | 0.004 | 0.007 |
| 12780 | survived | 0.009 | 0.012 |
| 12797 | survived | 0.005 | 0.013 |
| 12804 | survived | 0.003 | 0.007 |
| 12824 | survived | 0.03 | 0.03 |
| 12871 | survived | 0.006 | 0.018 |
| 12880 | survived | 0.009 | 0.007 |
| 12897 | survived | 0.01 | 0.013 |
| 12899 | survived | 0.002 | 0.007 |
| 12913 | survived | 0.007 | 0.009 |
| 12935 | survived | 0.005 | 0.008 |
| 12939 | survived | 0.006 | 0.014 |
| 12946 | survived | 0.042 | 0.025 |
| 12950 | survived | 0.017 | 0.026 |
| 12962 | survived | 0.007 | 0.01 |
| 12995 | survived | 0.003 | 0.008 |
| 12997 | survived | 0.014 | 0.017 |
| 12998 | survived | 0.086 | 0.038 |
| 13006 | survived | 0.004 | 0.01 |
| 13026 | survived | 0.04 | 0.023 |
| 13027 | survived | 0.004 | 0.01 |
| 13038 | survived | 0.002 | 0.006 |
| 13039 | survived | 0.002 | 0.008 |
| 13045 | survived | 0.006 | 0.007 |
| 13056 | survived | 0.005 | 0.01 |
| 13058 | survived | 0.054 | 0.039 |
| 13061 | survived | 0.008 | 0.008 |
| 13087 | survived | 0.003 | 0.016 |
| 13093 | survived | 0.004 | 0.013 |
| 13136 | died | 0.233 | 0.05 |
| 13196 | survived | 0.005 | 0.015 |
| 13198 | survived | 0.011 | 0.063 |
| 13203 | survived | 0.017 | 0.015 |
| 13248 | survived | 0.197 | 0.056 |
| 13261 | survived | 0.006 | 0.009 |
| 13266 | survived | 0.006 | 0.01 |
| 13273 | survived | 0.003 | 0.011 |
| 13279 | survived | 0.048 | 0.027 |
| 13285 | survived | 0.004 | 0.01 |
| 13297 | survived | 0.003 | 0.015 |
| 13303 | survived | 0.037 | 0.012 |
| 13309 | survived | 0.002 | 0.007 |
| 13349 | survived | 0.026 | 0.035 |
| 13365 | survived | 0.018 | 0.013 |
| 13372 | survived | 0.01 | 0.008 |
| 13386 | survived | 0.004 | 0.008 |
| 13388 | survived | 0.013 | 0.01 |
| 13399 | survived | 0.003 | 0.008 |
| 13409 | survived | 0.005 | 0.007 |
| 13417 | survived | 0.002 | 0.008 |
| 13425 | survived | 0.007 | 0.011 |
| 13438 | survived | 0.004 | 0.01 |
| 13443 | survived | 0.057 | 0.067 |
| 13446 | survived | 0.046 | 0.036 |
| 13454 | survived | 0.008 | 0.012 |
| 13465 | survived | 0.005 | 0.009 |
| 13468 | survived | 0.004 | 0.01 |
| 13483 | survived | 0.006 | 0.012 |
| 13484 | survived | 0.008 | 0.011 |
| 13489 | survived | 0.005 | 0.01 |
| 13533 | survived | 0.006 | 0.011 |
| 13534 | survived | 0.081 | 0.034 |
| 13546 | survived | 0.059 | 0.036 |
| 13594 | survived | 0.005 | 0.008 |
| 13603 | survived | 0.004 | 0.006 |
| 13610 | survived | 0.025 | 0.015 |
| 13613 | survived | 0.024 | 0.013 |
| 13625 | survived | 0.003 | 0.01 |
| 13654 | survived | 0.003 | 0.008 |
| 13658 | survived | 0.014 | 0.01 |
| 13688 | survived | 0.073 | 0.055 |
| 13694 | survived | 0.055 | 0.028 |
| 13715 | survived | 0.007 | 0.013 |
| 13725 | survived | 0.002 | 0.01 |
| 13731 | survived | 0.028 | 0.014 |
| 13732 | survived | 0.008 | 0.009 |
| 13737 | survived | 0.006 | 0.011 |
| 13741 | survived | 0.003 | 0.014 |
| 13742 | survived | 0.008 | 0.014 |
| 13748 | survived | 0.005 | 0.009 |
| 13757 | survived | 0.002 | 0.007 |
| 13759 | survived | 0.007 | 0.014 |
| 13768 | survived | 0.025 | 0.019 |
| 13770 | survived | 0.004 | 0.011 |
| 13775 | survived | 0.004 | 0.008 |
| 13782 | survived | 0.003 | 0.008 |
| 13798 | survived | 0.005 | 0.009 |
| 13815 | survived | 0.004 | 0.006 |
| 13832 | survived | 0.012 | 0.029 |
| 13844 | survived | 0.009 | 0.014 |
| 13848 | survived | 0.018 | 0.015 |
| 13857 | survived | 0.027 | 0.018 |
| 13858 | survived | 0.002 | 0.007 |
| 13872 | survived | 0.01 | 0.018 |
| 13902 | survived | 0.011 | 0.027 |
| 13920 | survived | 0.07 | 0.056 |
| 13921 | survived | 0.019 | 0.034 |
| 13935 | survived | 0.005 | 0.007 |
| 13956 | survived | 0.042 | 0.019 |
| 13959 | survived | 0.007 | 0.008 |
| 13963 | survived | 0.079 | 0.026 |
| 13970 | survived | 0.007 | 0.011 |
| 13987 | survived | 0.004 | 0.007 |
| 13991 | survived | 0.016 | 0.012 |
| 13995 | survived | 0.024 | 0.02 |
| 13996 | survived | 0.005 | 0.007 |
| 13997 | survived | 0.032 | 0.039 |
| 14002 | survived | 0.007 | 0.012 |
| 14003 | survived | 0.01 | 0.023 |
| 14015 | survived | 0.01 | 0.014 |
| 14022 | survived | 0.003 | 0.007 |
| 14025 | survived | 0.003 | 0.01 |
| 14026 | survived | 0.031 | 0.015 |
| 14032 | survived | 0.002 | 0.005 |
| 14039 | survived | 0.013 | 0.019 |
| 14065 | survived | 0.009 | 0.009 |
| 14084 | survived | 0.011 | 0.028 |
| 14087 | survived | 0.004 | 0.006 |
| 14102 | survived | 0.006 | 0.025 |
| 14115 | survived | 0.011 | 0.022 |
| 14121 | survived | 0.003 | 0.007 |
| 14128 | survived | 0.007 | 0.011 |
| 14130 | survived | 0.002 | 0.007 |
| 14132 | survived | 0.018 | 0.021 |
| 14139 | survived | 0.078 | 0.036 |
| 14145 | survived | 0.005 | 0.008 |
| 14147 | survived | 0.014 | 0.014 |
| 14148 | survived | 0.007 | 0.025 |
| 14161 | survived | 0.008 | 0.015 |
| 14162 | survived | 0.004 | 0.008 |
| 14164 | survived | 0.044 | 0.048 |
| 14198 | survived | 0.023 | 0.022 |
| 14204 | survived | 0.015 | 0.013 |
| 14206 | survived | 0.003 | 0.006 |
| 14211 | survived | 0.004 | 0.011 |
| 14231 | survived | 0.006 | 0.007 |
| 14234 | survived | 0.02 | 0.025 |
| 14248 | survived | 0.004 | 0.01 |
| 14253 | survived | 0.01 | 0.012 |
| 14260 | survived | 0.012 | 0.011 |
| 14270 | survived | 0.03 | 0.025 |
| 14276 | survived | 0.004 | 0.007 |
| 14280 | survived | 0.006 | 0.01 |
| 14285 | survived | 0.003 | 0.007 |
| 14297 | survived | 0.011 | 0.014 |
| 14299 | survived | 0.008 | 0.01 |
| 14309 | survived | 0.01 | 0.01 |
| 14321 | survived | 0.013 | 0.013 |
| 14323 | survived | 0.005 | 0.009 |
| 14329 | survived | 0.009 | 0.01 |
| 14333 | survived | 0.013 | 0.017 |
| 14337 | survived | 0.007 | 0.012 |
| 14344 | survived | 0.007 | 0.016 |
| 14345 | survived | 0.004 | 0.019 |
| 14362 | survived | 0.004 | 0.014 |
| 14363 | survived | 0.006 | 0.018 |
| 14370 | survived | 0.007 | 0.013 |
| 14378 | survived | 0.004 | 0.01 |
| 14389 | survived | 0.003 | 0.012 |
| 14393 | died | 0.16 | 0.057 |
| 14414 | survived | 0.165 | 0.035 |
| 14421 | survived | 0.005 | 0.01 |
| 14422 | survived | 0.017 | 0.012 |
| 14425 | survived | 0.013 | 0.014 |
| 14437 | survived | 0.024 | 0.013 |
| 14445 | survived | 0.026 | 0.012 |
| 14527 | survived | 0.002 | 0.008 |
| 14535 | survived | 0.02 | 0.019 |
| 14536 | survived | 0.011 | 0.025 |
| 14545 | survived | 0.004 | 0.01 |
| 14547 | survived | 0.003 | 0.007 |
| 14552 | survived | 0.002 | 0.009 |
| 14565 | survived | 0.004 | 0.009 |
| 14570 | survived | 0.009 | 0.012 |
| 14598 | survived | 0.034 | 0.027 |
| 14617 | survived | 0.017 | 0.014 |
| 14619 | survived | 0.002 | 0.006 |
| 14625 | survived | 0.004 | 0.013 |
| 14628 | survived | 0.02 | 0.011 |
| 14632 | survived | 0.038 | 0.021 |
| 14638 | survived | 0.006 | 0.008 |
| 14648 | survived | 0.005 | 0.009 |
| 14657 | survived | 0.004 | 0.014 |
| 14684 | survived | 0.005 | 0.007 |
| 14698 | survived | 0.009 | 0.015 |
| 14712 | survived | 0.004 | 0.018 |
| 14713 | survived | 0.005 | 0.009 |
| 14719 | survived | 0.003 | 0.007 |
| 14728 | survived | 0.005 | 0.013 |
| 14737 | survived | 0.013 | 0.016 |
| 14741 | survived | 0.009 | 0.009 |
| 14743 | survived | 0.009 | 0.017 |
| 14749 | survived | 0.01 | 0.019 |
| 14759 | survived | 0.011 | 0.014 |
| 14764 | survived | 0.004 | 0.01 |

SUPPLEMENTAL TABLE 6
Predicted probabilities for obstructive CAD requiring
revas-cularization within 30 days using the minimal
and the full model (of a total of 33 models)

| rapID | outcome | minimal_model | full_model |
|---|---|---|---|
| 4 | obstructive | 0.811 | 0.794 |
| 13 | non-obstructive | 0.802 | 0.791 |
| 21 | obstructive | 0.769 | 0.759 |
| 31 | non-obstructive | 0.726 | 0.711 |
| 49 | non-obstructive | 0.526 | 0.486 |
| 54 | obstructive | 0.312 | 0.384 |
| 72 | obstructive | 0.437 | 0.456 |
| 77 | obstructive | 0.786 | 0.766 |
| 78 | non-obstructive | 0.767 | 0.703 |
| 95 | obstructive | 0.808 | 0.799 |
| 98 | non-obstructive | 0.655 | 0.656 |
| 124 | obstructive | 0.582 | 0.638 |
| 129 | obstructive | 0.756 | 0.743 |
| 155 | non-obstructive | 0.644 | 0.635 |
| 167 | non-obstructive | 0.602 | 0.675 |
| 175 | non-obstructive | 0.407 | 0.434 |
| 186 | non-obstructive | 0.357 | 0.398 |
| 197 | obstructive | 0.797 | 0.771 |
| 207 | obstructive | 0.476 | 0.503 |
| 273 | obstructive | 0.704 | 0.664 |
| 301 | obstructive | 0.475 | 0.528 |
| 338 | obstructive | 0.748 | 0.735 |
| 350 | non-obstructive | 0.732 | 0.724 |
| 363 | obstructive | 0.796 | 0.775 |
| 370 | obstructive | 0.834 | 0.812 |
| 374 | non-obstructive | 0.601 | 0.616 |
| 399 | obstructive | 0.492 | 0.525 |
| 415 | obstructive | 0.757 | 0.752 |
| 431 | obstructive | 0.672 | 0.7 |
| 459 | non-obstructive | 0.536 | 0.587 |
| 463 | non-obstructive | 0.841 | 0.82 |
| 468 | obstructive | 0.749 | 0.737 |
| 483 | non-obstructive | 0.591 | 0.623 |
| 511 | obstructive | 0.611 | 0.615 |
| 539 | obstructive | 0.898 | 0.827 |
| 577 | non-obstructive | 0.881 | 0.851 |
| 587 | non-obstructive | 0.402 | 0.464 |
| 606 | non-obstructive | 0.334 | 0.383 |
| 611 | non-obstructive | 0.621 | 0.615 |
| 645 | obstructive | 0.819 | 0.788 |
| 647 | non-obstructive | 0.65 | 0.739 |
| 650 | obstructive | 0.799 | 0.803 |
| 666 | non-obstructive | 0.501 | 0.515 |
| 698 | obstructive | 0.456 | 0.429 |
| 705 | obstructive | 0.585 | 0.596 |
| 720 | non-obstructive | 0.765 | 0.747 |
| 733 | obstructive | 0.887 | 0.847 |
| 780 | non-obstructive | 0.828 | 0.795 |
| 781 | obstructive | 0.592 | 0.543 |
| 789 | non-obstructive | 0.664 | 0.656 |
| 790 | obstructive | 0.769 | 0.758 |
| 840 | obstructive | 0.904 | 0.829 |
| 848 | obstructive | 0.822 | 0.816 |
| 850 | obstructive | 0.564 | 0.518 |
| 867 | obstructive | 0.942 | 0.905 |
| 881 | non-obstructive | 0.614 | 0.563 |
| 885 | obstructive | 0.84 | 0.813 |
| 926 | non-obstructive | 0.526 | 0.569 |
| 935 | obstructive | 0.619 | 0.612 |
| 957 | obstructive | 0.738 | 0.687 |
| 970 | obstructive | 0.42 | 0.459 |
| 1000 | non-obstructive | 0.34 | 0.385 |
| 1003 | obstructive | 0.833 | 0.81 |
| 1037 | obstructive | 0.547 | 0.559 |
| 1038 | obstructive | 0.831 | 0.803 |
| 1067 | non-obstructive | 0.755 | 0.741 |
| 1082 | non-obstructive | 0.5 | 0.531 |
| 1084 | non-obstructive | 0.744 | 0.737 |
| 1085 | obstructive | 0.619 | 0.637 |
| 1091 | non-obstructive | 0.622 | 0.648 |
| 1099 | non-obstructive | 0.741 | 0.707 |
| 1112 | obstructive | 0.893 | 0.87 |
| 1190 | non-obstructive | 0.338 | 0.358 |
| 1195 | obstructive | 0.709 | 0.664 |
| 1242 | obstructive | 0.948 | 0.918 |
| 1243 | obstructive | 0.772 | 0.722 |
| 1310 | non-obstructive | 0.808 | 0.784 |
| 1342 | obstructive | 0.808 | 0.811 |
| 1344 | obstructive | 0.655 | 0.602 |
| 1424 | obstructive | 0.494 | 0.519 |
| 1471 | obstructive | 0.893 | 0.828 |
| 1476 | non-obstructive | 0.395 | 0.448 |
| 1483 | non-obstructive | 0.426 | 0.447 |
| 1518 | obstructive | 0.788 | 0.782 |
| 1537 | obstructive | 0.75 | 0.654 |
| 1553 | non-obstructive | 0.497 | 0.516 |
| 1596 | non-obstructive | 0.808 | 0.775 |
| 1604 | non-obstructive | 0.67 | 0.67 |
| 1607 | non-obstructive | 0.488 | 0.552 |
| 1621 | obstructive | 0.7 | 0.703 |
| 1650 | obstructive | 0.745 | 0.719 |
| 1658 | obstructive | 0.69 | 0.714 |
| 1692 | non-obstructive | 0.667 | 0.656 |
| 1697 | non-obstructive | 0.475 | 0.546 |
| 1757 | obstructive | 0.707 | 0.712 |
| 1760 | obstructive | 0.548 | 0.562 |
| 1772 | non-obstructive | 0.221 | 0.294 |
| 1775 | obstructive | 0.794 | 0.79 |
| 1783 | obstructive | 0.689 | 0.64 |
| 1791 | obstructive | 0.721 | 0.698 |
| 1843 | non-obstructive | 0.631 | 0.628 |
| 1854 | obstructive | 0.603 | 0.604 |
| 1892 | obstructive | 0.825 | 0.807 |
| 1895 | non-obstructive | 0.807 | 0.805 |
| 1905 | obstructive | 0.637 | 0.66 |
| 1907 | obstructive | 0.734 | 0.732 |
| 1916 | non-obstructive | 0.403 | 0.456 |
| 1931 | obstructive | 0.644 | 0.681 |
| 1937 | obstructive | 0.353 | 0.409 |
| 1954 | non-obstructive | 0.685 | 0.666 |
| 1992 | non-obstructive | 0.58 | 0.585 |
| 2039 | obstructive | 0.889 | 0.859 |
| 2043 | non-obstructive | 0.343 | 0.443 |
| 2054 | obstructive | 0.761 | 0.779 |
| 2058 | non-obstructive | 0.645 | 0.643 |
| 2102 | non-obstructive | 0.552 | 0.733 |
| 2112 | obstructive | 0.706 | 0.647 |
| 2153 | obstructive | 0.475 | 0.513 |
| 2178 | obstructive | 0.666 | 0.65 |
| 2187 | obstructive | 0.841 | 0.793 |
| 2218 | obstructive | 0.883 | 0.854 |
| 2266 | obstructive | 0.827 | 0.796 |
| 2270 | obstructive | 0.719 | 0.678 |
| 2281 | obstructive | 0.833 | 0.852 |
| 2318 | non-obstructive | 0.257 | 0.322 |
| 2382 | obstructive | 0.744 | 0.727 |
| 2421 | obstructive | 0.583 | 0.599 |
| 2452 | non-obstructive | 0.521 | 0.546 |
| 2464 | obstructive | 0.773 | 0.762 |
| 2503 | obstructive | 0.873 | 0.846 |
| 2510 | obstructive | 0.614 | 0.562 |
| 2511 | non-obstructive | 0.39 | 0.42 |
| 2535 | non-obstructive | 0.455 | 0.512 |
| 2606 | obstructive | 0.652 | 0.672 |
| 2613 | obstructive | 0.431 | 0.511 |
| 2654 | obstructive | 0.674 | 0.75 |
| 2662 | obstructive | 0.87 | 0.816 |
| 2691 | obstructive | 0.471 | 0.507 |
| 2694 | obstructive | 0.648 | 0.581 |
| 2700 | non-obstructive | 0.476 | 0.449 |
| 2717 | obstructive | 0.587 | 0.631 |
| 2733 | non-obstructive | 0.812 | 0.795 |
| 2735 | non-obstructive | 0.336 | 0.393 |
| 2766 | obstructive | 0.759 | 0.777 |
| 2774 | obstructive | 0.731 | 0.695 |
| 2798 | obstructive | 0.725 | 0.809 |
| 2828 | obstructive | 0.824 | 0.8 |
| 2848 | obstructive | 0.832 | 0.802 |
| 2849 | non-obstructive | 0.796 | 0.769 |
| 2896 | obstructive | 0.75 | 0.72 |
| 2909 | non-obstructive | 0.764 | 0.755 |
| 2953 | obstructive | 0.754 | 0.741 |
| 2955 | obstructive | 0.757 | 0.751 |
| 2989 | non-obstructive | 0.463 | 0.496 |
| 2990 | non-obstructive | 0.5 | 0.531 |
| 3000 | obstructive | 0.857 | 0.834 |
| 3001 | obstructive | 0.708 | 0.707 |
| 3074 | obstructive | 0.433 | 0.471 |
| 3154 | obstructive | 0.73 | 0.721 |
| 3158 | obstructive | 0.805 | 0.752 |
| 3182 | non-obstructive | 0.874 | 0.858 |
| 3187 | obstructive | 0.732 | 0.68 |
| 3213 | obstructive | 0.652 | 0.61 |
| 3230 | obstructive | 0.757 | 0.767 |
| 3286 | non-obstructive | 0.436 | 0.496 |
| 3381 | obstructive | 0.894 | 0.861 |
| 10067 | obstructive | 0.781 | 0.762 |
| 10069 | obstructive | 0.801 | 0.738 |
| 10127 | non-obstructive | 0.785 | 0.776 |
| 10164 | obstructive | 0.739 | 0.733 |
| 10208 | obstructive | 0.755 | 0.766 |
| 10253 | non-obstructive | 0.562 | 0.586 |
| 10295 | obstructive | 0.726 | 0.717 |
| 10328 | obstructive | 0.583 | 0.605 |
| 10333 | obstructive | 0.647 | 0.65 |
| 10406 | non-obstructive | 0.759 | 0.744 |
| 10417 | non-obstructive | 0.635 | 0.639 |
| 10419 | obstructive | 0.806 | 0.803 |
| 10435 | non-obstructive | 0.604 | 0.65 |
| 10451 | obstructive | 0.713 | 0.707 |
| 10481 | non-obstructive | 0.745 | 0.723 |
| 10487 | non-obstructive | 0.655 | 0.66 |
| 10496 | obstructive | 0.836 | 0.802 |
| 10515 | non-obstructive | 0.795 | 0.795 |
| 10554 | non-obstructive | 0.827 | 0.796 |
| 10583 | non-obstructive | 0.664 | 0.682 |
| 10628 | non-obstructive | 0.62 | 0.617 |
| 10654 | obstructive | 0.857 | 0.846 |
| 10665 | obstructive | 0.609 | 0.634 |
| 10685 | obstructive | 0.62 | 0.582 |
| 10697 | obstructive | 0.729 | 0.729 |
| 10701 | obstructive | 0.609 | 0.598 |
| 10747 | non-obstructive | 0.711 | 0.731 |
| 10785 | obstructive | 0.653 | 0.666 |
| 10791 | obstructive | 0.404 | 0.455 |
| 10864 | obstructive | 0.79 | 0.773 |
| 10945 | obstructive | 0.638 | 0.684 |
| 10968 | non-obstructive | 0.55 | 0.557 |
| 10981 | obstructive | 0.805 | 0.817 |
| 11002 | obstructive | 0.888 | 0.797 |
| 11069 | obstructive | 0.724 | 0.709 |
| 11110 | obstructive | 0.434 | 0.494 |
| 11175 | non-obstructive | 0.59 | 0.601 |
| 11184 | non-obstructive | 0.484 | 0.537 |
| 11235 | non-obstructive | 0.293 | 0.343 |
| 11240 | obstructive | 0.793 | 0.748 |
| 11282 | obstructive | 0.863 | 0.874 |
| 11327 | obstructive | 0.728 | 0.741 |
| 11340 | non-obstructive | 0.573 | 0.599 |
| 11348 | obstructive | 0.477 | 0.535 |
| 11365 | obstructive | 0.343 | 0.396 |
| 11377 | non-obstructive | 0.857 | 0.829 |
| 11429 | obstructive | 0.41 | 0.448 |
| 11442 | obstructive | 0.421 | 0.466 |
| 11464 | obstructive | 0.812 | 0.789 |
| 11477 | obstructive | 0.473 | 0.496 |
| 11492 | non-obstructive | 0.778 | 0.718 |
| 11495 | obstructive | 0.675 | 0.719 |
| 11499 | obstructive | 0.796 | 0.777 |
| 11554 | obstructive | 0.748 | 0.707 |
| 11613 | obstructive | 0.754 | 0.737 |
| 11639 | obstructive | 0.61 | 0.635 |
| 11641 | obstructive | 0.755 | 0.754 |
| 11655 | obstructive | 0.903 | 0.877 |
| 11658 | obstructive | 0.875 | 0.858 |
| 11660 | obstructive | 0.792 | 0.782 |
| 11712 | obstructive | 0.858 | 0.817 |
| 11813 | obstructive | 0.744 | 0.767 |
| 11832 | non-obstructive | 0.628 | 0.649 |
| 11848 | obstructive | 0.829 | 0.806 |
| 11893 | obstructive | 0.852 | 0.813 |
| 11897 | obstructive | 0.519 | 0.545 |
| 11925 | obstructive | 0.727 | 0.72 |
| 11944 | obstructive | 0.817 | 0.78 |
| 11947 | obstructive | 0.647 | 0.687 |
| 11955 | obstructive | 0.664 | 0.688 |
| 12011 | non-obstructive | 0.559 | 0.598 |
| 12014 | non-obstructive | 0.734 | 0.733 |
| 12034 | obstructive | 0.74 | 0.742 |
| 12064 | obstructive | 0.693 | 0.705 |
| 12070 | obstructive | 0.72 | 0.737 |
| 12088 | obstructive | 0.868 | 0.834 |
| 12107 | non-obstructive | 0.604 | 0.622 |
| 12226 | non-obstructive | 0.608 | 0.595 |
| 12228 | obstructive | 0.431 | 0.48 |
| 12262 | obstructive | 0.764 | 0.756 |
| 12264 | obstructive | 0.88 | 0.855 |
| 12266 | obstructive | 0.541 | 0.569 |
| 12274 | obstructive | 0.824 | 0.805 |
| 12330 | obstructive | 0.95 | 0.925 |
| 12340 | non-obstructive | 0.77 | 0.762 |
| 12343 | obstructive | 0.473 | 0.535 |
| 12363 | obstructive | 0.679 | 0.682 |
| 12379 | non-obstructive | 0.326 | 0.381 |
| 12408 | obstructive | 0.825 | 0.79 |
| 12437 | obstructive | 0.614 | 0.573 |
| 12452 | obstructive | 0.874 | 0.87 |
| 12472 | obstructive | 0.734 | 0.73 |
| 12567 | obstructive | 0.955 | 0.939 |
| 12633 | non-obstructive | 0.661 | 0.704 |
| 12656 | obstructive | 0.657 | 0.608 |
| 12666 | non-obstructive | 0.664 | 0.675 |
| 12682 | obstructive | 0.777 | 0.785 |
| 12700 | obstructive | 0.737 | 0.729 |
| 12705 | obstructive | 0.881 | 0.854 |
| 12752 | obstructive | 0.852 | 0.785 |
| 12843 | obstructive | 0.736 | 0.731 |
| 12873 | non-obstructive | 0.705 | 0.7 |
| 12883 | obstructive | 0.515 | 0.539 |
| 12914 | obstructive | 0.907 | 0.874 |
| 12994 | obstructive | 0.877 | 0.845 |
| 13012 | obstructive | 0.848 | 0.803 |
| 13021 | obstructive | 0.649 | 0.647 |
| 13035 | non-obstructive | 0.664 | 0.662 |
| 13040 | obstructive | 0.597 | 0.538 |
| 13047 | non-obstructive | 0.675 | 0.728 |
| 13113 | obstructive | 0.833 | 0.823 |
| 13131 | non-obstructive | 0.722 | 0.712 |
| 13190 | non-obstructive | 0.481 | 0.543 |
| 13263 | obstructive | 0.82 | 0.812 |
| 13269 | obstructive | 0.837 | 0.8 |
| 13334 | obstructive | 0.572 | 0.606 |
| 13351 | obstructive | 0.701 | 0.723 |
| 13356 | non-obstructive | 0.596 | 0.614 |
| 13362 | obstructive | 0.829 | 0.798 |
| 13363 | obstructive | 0.8 | 0.781 |
| 13375 | obstructive | 0.554 | 0.569 |
| 13469 | non-obstructive | 0.415 | 0.457 |
| 13497 | obstructive | 0.447 | 0.459 |
| 13551 | obstructive | 0.713 | 0.695 |
| 13552 | obstructive | 0.909 | 0.862 |
| 13585 | obstructive | 0.618 | 0.622 |
| 13607 | obstructive | 0.851 | 0.81 |
| 13610 | non-obstructive | 0.617 | 0.624 |
| 13618 | obstructive | 0.824 | 0.744 |
| 13629 | obstructive | 0.732 | 0.665 |
| 13646 | obstructive | 0.454 | 0.503 |
| 13651 | non-obstructive | 0.407 | 0.39 |
| 13655 | obstructive | 0.636 | 0.661 |
| 13702 | obstructive | 0.831 | 0.786 |
| 13719 | obstructive | 0.878 | 0.848 |
| 13721 | obstructive | 0.869 | 0.829 |
| 13728 | non-obstructive | 0.565 | 0.588 |
| 13747 | non-obstructive | 0.695 | 0.684 |
| 13768 | obstructive | 0.82 | 0.794 |
| 13771 | obstructive | 0.847 | 0.832 |
| 13785 | obstructive | 0.477 | 0.509 |
| 13798 | obstructive | 0.632 | 0.576 |
| 13805 | obstructive | 0.877 | 0.849 |
| 13843 | non-obstructive | 0.702 | 0.705 |
| 13891 | non-obstructive | 0.594 | 0.687 |
| 13908 | non-obstructive | 0.693 | 0.697 |
| 13947 | non-obstructive | 0.722 | 0.849 |
| 14002 | non-obstructive | 0.365 | 0.42 |
| 14015 | obstructive | 0.766 | 0.762 |
| 14018 | non-obstructive | 0.568 | 0.58 |
| 14024 | non-obstructive | 0.766 | 0.748 |
| 14101 | non-obstructive | 0.299 | 0.372 |
| 14194 | obstructive | 0.407 | 0.502 |
| 14207 | obstructive | 0.661 | 0.658 |
| 14246 | obstructive | 0.719 | 0.763 |
| 14247 | obstructive | 0.538 | 0.561 |
| 14296 | obstructive | 0.647 | 0.595 |
| 14297 | obstructive | 0.732 | 0.707 |
| 14327 | obstructive | 0.634 | 0.652 |
| 14359 | non-obstructive | 0.91 | 0.89 |
| 14387 | obstructive | 0.711 | 0.709 |
| 14464 | obstructive | 0.861 | 0.82 |
| 14477 | non-obstructive | 0.457 | 0.496 |
| 14487 | non-obstructive | 0.722 | 0.697 |
| 14536 | non-obstructive | 0.417 | 0.485 |
| 14540 | obstructive | 0.857 | 0.812 |
| 14551 | obstructive | 0.794 | 0.771 |
| 14558 | obstructive | 0.693 | 0.699 |
| 14570 | obstructive | 0.615 | 0.654 |
| 14601 | obstructive | 0.831 | 0.775 |
| 14632 | obstructive | 0.663 | 0.666 |

1. A computer-implemented method for predicting the risk of an adverse event of a patient presenting with suspected acute coronary syndrome, comprising the steps of
a) receiving data for a set of parameters obtained from the patient at a processing unit, wherein said set of parameters comprises at least five of the following parameters
i. the amount of a cardiac Troponin in a first sample obtained from said patient at presentation,
ii. the amount of said cardiac Troponin in a second sample obtained from said patient within 30 minutes to 6 hours after the first sample,
iii. the amount of CRP (C-reactive protein) in a sample from the patient,
iv. at least one parameter for the patient's renal function selected from the group consisting of the amount of urea in a sample from the patient, the patient's GFR, and the patient's serum creatinine amount,
v. the amount of sodium in a sample from the patient,
vi. the amount of hemoglobin in a sample from the patient,
vii. the patient's thrombocyte level,
viii. the patient's age,
ix. the patient's gender, and
x. the presence or absence of a normal ECG in said patient,
b) carrying out at the processing unit an analysis of the set of parameters, wherein said analysis comprises calculating a score for predicting the risk of an adverse event of said patient based on the set of parameters received in step a), and
c) providing information on the score calculated in step b), thereby predicting the risk of an adverse event.

2. The method of claim 1, wherein the adverse event is death and/or wherein the risk of an adverse event within about 180 to about 365 days is predicted.

3. The method of claim 1, wherein the score is indicative for the likelihood of the patient to suffer from an adverse event.

4. The method of claim 1, wherein in step a) data on at least five of the following parameters are received: the amount of a cardiac Troponin in a first sample obtained from said patient at presentation; the amount of said cardiac Troponin in a second sample; the amount of CRP (C-reactive protein) in a sample from the patient, the amount of creatinine in a serum sample from the patient, the amount of sodium in a sample from the patient, the amount of hemoglobin in a sample from the patient, the patient's thrombocyte level, information on the patient's age, information on the patient's gender, and information on the presence or absence of a normal ECG in said patient.

5. The method of claim 1, wherein in step a) data on at least the following parameters are obtained: the amount of a cardiac Troponin in a first sample obtained from said patient at presentation; the amount of said cardiac Troponin in a second sample; the amount of CRP (C-reactive protein) in a sample from the patient; the patient's creatinine value; information on the patient's age; and information on the patient's gender.

6. A method of predicting the risk of an adverse event of a patient presenting with suspected acute coronary syndrome, comprising the steps of
a) carrying out at least five of the following steps a1) to a10):
a1) determining the amount of a cardiac Troponin in a first sample obtained from said patient at presentation,
a2) determining the amount of said cardiac Troponin in a second sample obtained from said patient within 30 minutes to six hours after the first sample,
a3) the amount of CRP (C-reactive protein) in a sample from the patient,
a4) determining at least one parameter for the patient's renal function selected from the group consisting of the amount of urea nitrogen in a (blood) sample from the patient, the patient's GFR, and the patient's serum creatinine amount,
a5) determining the amount of sodium in a sample from the patient,
a6) determining the amount of hemoglobin in a sample from the patient,
a7) determining the patient's thrombocyte level,
a8) providing information on the patient's age,
a9) providing information on the patient's gender, and
a10) providing information on the presence or absence of a normal ECG in said patient,
b) calculating a score for predicting the risk of an adverse event of said patient based on the information obtained in step a), and
c) predicting the risk of the patient of an adverse event based on the score calculated in step b).

7. A computer-implemented method for predicting the need of myocardial revascularization of a patient presenting with suspected acute coronary syndrome, comprising the steps of
a) receiving data for a set of parameters obtained from the patient at a processing unit, wherein said set of parameters comprises at least eight of the following parameters:
i. the amount of a cardiac Troponin in a first sample obtained from said patient at presentation,
ii. the amount of said cardiac Troponin in a second sample obtained from said patient within 30 minutes to 6 hours after the first sample,
iii. the patient's age,
iv. the patient's gender,
v. a parameter for the patient's renal function selected from the group consisting of the patient's GFR, the patient's serum creatinine amount, and the patient's urea or blood urea nitrogen (BUN) amount,
vi. the presence or absence of chest symptoms in said patient,
vii. the presence or absence of dyspnea in said patient,
viii. the presence or absence of a normal ECG in said patient, and
ix. information on the patient's past medical history, comprising at least one of the following: information on the patient's history of diabetes, information on the patient's history of smoking, and information on the patient's history of coronary heart disease,
b) carrying out at the processing unit an analysis of the set of parameters, wherein said analysis comprises calculating a score for predicting the need of myocardial revascularization of said patient based on the set of parameters received in step a), and
c) providing information on the score calculated in step b), thereby predicting the need of myocardial revascularization of said patient.

8. The method of claim 7, wherein the myocardial revascularization is due to obstructive coronary artery disease.

9. The method of claim 7, wherein the need of myocardial revascularization within about 30 days is predicted.

10. The method of claim 7, wherein in step a) data on at least eight of the following parameters are received: the amount of a cardiac Troponin in a first sample obtained from said patient at presentation; the amount of said cardiac Troponin in a second sample; the patient's serum creatinine amount; the presence or absence of chest symptoms in said patient, the presence or absence of dyspnea in said patient, information on the patient's sex (gender); and information on the patient's past medical history comprising at least one of the following: information on the patient's history of diabetes, information on the patient's history of smoking, information on the patient's history of coronary heart disease, and the presence or absence of a normal ECG in said patient.

11. The method of claim 7, wherein in step a) data on at least the eight following parameters are obtained: the amount of a cardiac Troponin in a first sample obtained from said patient at presentation; the amount of said cardiac Troponin in a second sample; information on the patient's sex (gender), information on the patient's age, creatinine, information on the patient's history of coronary artery disease, information on the patient's history of smoking (past or active), and information on the presence of chest symptoms in the patient.

12. The method of claim 7, wherein the myocardial revascularization is one of the following:
a. invasive coronary angiography with percutaneous coronary intervention with or without stenting,
b. invasive coronary angiography with lesion(s) suitable for revascularization and attempted percutaneous coronary intervention with or without stenting,
c. invasive coronary angiography with lesion(s) suitable for revascularization and attempted but failed or unsuccessful percutaneous coronary intervention,
d. invasive coronary angiography with relevant lesion(s) unsuitable for revascularization and subsequently conservative medical treatment without percutaneous coronary intervention,
e. invasive coronary angiography with lesion(s) suitable for revascularization and immediate transfer for coronary bypass surgery
f. invasive coronary angiography with lesion(s) suitable for revascularization and recommendation for coronary bypass surgery, or
g. invasive coronary angiography with lesion(s) planned for revascularization.

13. A method of predicting the need of myocardial revascularization of a patient presenting with suspected acute coronary syndrome, comprising
a) carrying out at least eight of the following steps a1) to a9):
a1) determining the amount of a cardiac Troponin in a first sample obtained from said patient at presentation,
a2) determining the amount of said cardiac Troponin in a second sample obtained from said patient within 30 minutes to six hours after the first sample,
a3) providing information on the patient's gender,
a4) providing information on the patient's age,
a5) providing information on a parameter for the patient's renal function selected from the group consisting of the patient's GFR, the patient's serum creatinine amount, and the patient's urea or blood urea nitrogen (BUN) amount,
a6) providing information on the presence or absence of chest symptoms in said patient,
a7) providing information on the presence or absence of dyspnea in said patient,
a8) providing information on the presence or absence of a normal ECG in said patient, and
a9) providing information on the patient's past medical history, comprising at least one of the following: information on the patient's history of diabetes, information on the patient's history of smoking, and information on the patient's history of coronary artery disease,
b) calculating a score for predicting the need of myocardial revascularization of said patient based on the information obtained in step a), and
c) predicting the need of myocardial revascularization of the patient event based on the score calculated in step b).

14. The method of claim 7, wherein the score is indicative for the likelihood of the patient to require myocardial revascularization.

15. The method of claim 7, wherein the sample is a blood, serum or plasma sample, and/or wherein the calculated score is shown on a display.

16. A device for predicting of the risk of an adverse event or predicting the need of myocardial revascularization, said device comprising a processing unit, and a computer program including computer-executable instructions, wherein said instructions, when executed by the processing unit, causes the processing unit to perform the computer-implemented method according to claim 1.

17. The method of claim 7, wherein the information on the patient's history of coronary heart disease comprises information on the patient's history of myocardial revascularization.

18. The method of claim 10, wherein information on the patient's past medical history comprises information on whether the patient has a normal or abnormal ECG.

19. The method of claim 12, wherein the myocardial revascularization is percutaneous coronary intervention or coronary bypass surgery.
